(19) United States
(12) Patent Application Publication
(10) Pub. No.: US 2023/0303551 A1
(43) Pub. Date: Sep. 28, 2023

(54) N-CYCLYL-SULFONAMIDES USEFUL FOR INHIBITING RAF

(71) Applicant: ALBERT EINSTEIN COLLEGE OF MEDICINE, Bronx, NY (US)

(72) Inventors: Evripidis GAVATHIOTIS, Roslyn, NY (US); Bogos AGIANIAN, Bayside, NY (US)

(21) Appl. No.: 18/041,262

(22) PCT Filed: Aug. 13, 2021
(86) PCT No.: PCT/US2021/045877

§ 371 Date: Feb. 10, 2023

Related U.S. Application Data
(60) Provisional application No. 63/065,026, filed on Aug. 13, 2020.

(51) Int. Cl. C07D 417/04; A61K 45/06; C07D 417/14

(52) U.S. Cl. CPC C07D 417/04; A61K 45/06; C07D 417/14

(57) ABSTRACT

This disclosure provides compounds and pharmaceutically acceptable salts thereof of Formula I (Formula I) The variables, e.g. R¹-R⁴, and Z are defined herein. Y is Y is (II). Certain compounds of Formula I are highly potent against resistant tumor cell el lines driven by BRAF^{V600E} monomer melanoma cells (A375 or SK-MEL-239), p61-BRAF^{V600E} dimer splice variant melanoma cells (SK-MEL-239-C4) and colorectal (RKO) and lung cancer (A549) cells, and at the same time display a highly desirable pharmacological profile in a mice tumor model.

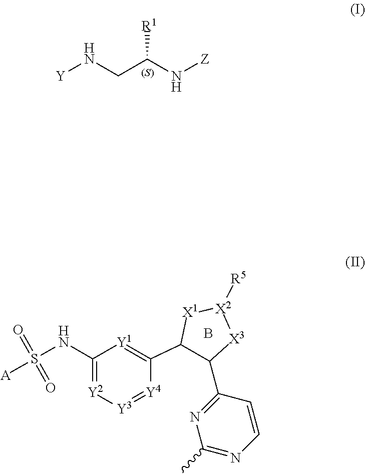

### CROSS REFERENCE TO RELATED APPLICATION

This application claims priority of U.S. Prov. Appl. No. 63/065,026, filed Aug. 13, 2020, which is hereby incorporated by reference in its entirety.

### FIELD OF THE DISCLOSURE

The disclosure is directed to compounds of Formula I, described herein, pharmaceutical compositions of compounds of Formula I, and methods of using compounds of Formula I to treat cancer, particularly cancers dependent on the RAF family, including cancers dependent on wild type BRAF dimers, mutant BRAF⁷⁶⁰⁰Emonomers, and mutant BRAF dimers, such as BRAF^{V600E}dimers.

### BACKGROUND

The RAS-RAF-MEK-ERK signaling pathway (Extracellular signal Related Kinase or ERK signaling) regulates mammalian cell growth, proliferation, and survival. This pathway is normally activated by growth factor receptor signaling that promotes activation of RAS at the plasma membrane. RAF kinases (ARAF, BRAF and CRAF isoforms) are subsequently recruited at the membrane by interaction with the active form of RAS bound to GTP, leading to a cascade of phosphorylation and activation steps of downstream kinases MEK1/2 and ERK1/2. Aberrant activation of ERK signaling is a hallmark of many cancers most commonly due to mutations of RAS and BRAF. BRAF mutants are found in up to 9% of all human cancers and over 60% of melanoma.

Cancers dependent on dimers of RAF family, include cancers dependent on wild type BRAF, BRAF^{V600E}, BRAF splice variants (including p61BRAF) and BRAF fusions, and BRAF dimers belonging to Class II and Class III. Additional BRAF mutations associated with cancer include R4621, 1463S, G464V, G464E, G466A, G466E, G466V, G469A, G469E, D594V, F595L, G596R, L597V, L597R, T5991, V600D, V600K, V600R, T1 19S, and K601E. Specifically, these cancers include melanoma, thyroid, non-small cell lung cancer, colorectal, ovarian, pancreatic, prostate, gastric, endometrial, hairy cell leukemia pediatric-low grade gliomas, BRAF^{V600E} gliomas, central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system).

RAF proteins activate ERK signaling as homo and hetero-dimers in the presence of active RAS. In contrast, BRAF^{V600E} can activate ERK signaling independent of RAS as an active monomer. Drug development efforts have yielded three FDA-approved RAF inhibitors, vemurafenib, dabrafenib and encorafenib that show good efficacy in patients with BRAF^{V600E} melanoma tumors. These drugs have elicited remarkable responses and improved survival of melanoma patients with BRAF^{V600E} tumors, but their acquired resistance and poor pharmacological properties (low residence time) limits their effectiveness, resulting in relapse of patients within ˜12 months. Although the current standard of care for these patients is the combination of a BRAF inhibitor with MEK inhibitors (e.g. trametinib, cobimetinib or binimetinib), these patients gain survival for a few months but eventually endure incomplete inhibition of oncogenic BRAF signaling. Eventually, only 10% of melanoma patients with BRAF^{V600E} under BRAF inhibitor treatment achieve a complete response.

Remarkably, the response rates to RAF inhibitors are dramatically lower for colorectal and thyroid cancer patients with BRAF^{V600E} ; almost 95% of colorectal cases are intrinsically resistant to vemurafenib (VEM) and up to 70% of thyroid cases to dabrafenib (DAB). Recent clinical studies showed that in BRAF^{V600E} melanoma, the overall response rate (ORR) is increased to ˜65% when patients receive BRAFi/MEKi treatment, but the same combination gives an ORR of only ˜12% in BRAF^{V600E} colorectal patients. Moreover, in about 20-30% of clinical melanoma tumors intrinsic resistance is driven by a constitutive p61-BRAF^{V600E} dimer splice variant. Therefore, there is urgent clinical need to develop novel BRAF inhibitors that can effectively target resistant BRAF^{V600E}-dependent tumors or tumors dependent on other oncogenic BRAF species such BRAF splice variants (including p61BRAF), BRAF fusions, and wild type of BRAF mutants belonging to Class II and Class III, which are not potently inhibited by current FDA-approved inhibitors.

### SUMMARY

This disclosure provides compounds of Formula I. Certain compounds of Formula I are highly potent against resistant tumor cell lines driven by BRAF^{V600E} monomer melanoma cells (A375 or SK-MEL-239), p61-BRAF^{V600E} dimer splice variant melanoma cells (SK-MEL-239-C4) and colorectal (RKO) and lung cancer (A549) cells, and at the same time display a highly desirable pharmacological profile in a mice tumor model. DABK, described below, is a compound of Formula I having these features. Compounds of Formula I are useful for treating a range of BRAF-dependent tumors, and other disorders in which RAS-RAF-MEK-ERK signaling plays a role, including tumors expressing BRAF mutations, alone or in combination treatment with other FDA-approved therapeutics.

This disclosure provides novel RAF inhibitors useful for treating cancer.

The disclosure provides a compound of Formula I

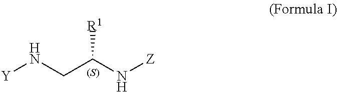

or a pharmaceutically acceptable salt thereof. Within Formula I the variables, e.g. R¹-R⁴, Y, and Z have the following definitions.

R¹ is hydrogen, —Cₙ, —CₘNH₂, —NHCₙ, or —CₘC═NH, Cₘ and Cₙ are independently chosen at each occurrence, where Cₘ is an alkylene or alkenylene group and Cₙ is an alkyl or alkenyl group, Cₘ and Cₙ each having the indicated number of carbon atoms and the requisite number of hydrogen atoms, and m and n are an integer from 1 to 6.
Y is

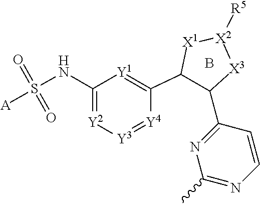

A is Ring A is C₃-C₇cycloalkyl, phenyl, or a 5-6 membered heterocycle having 1 or 2 heteroatoms independently selected from N, O, and S, each of which Ring A is optionally substituted, or A is a mono- or di-(C₁-C₆alkyl)amino.
Ring B is a heteroaryl ring, with at least one heteroatom;
X¹ is N or C;
X² is S or C;
X³ is S, O, or N;
Y¹, Y², Y³, and Y⁴ are independently N or CR⁶, where 0 or 1 of Y¹, Y², Y³, and Y⁴ are N;
Z is

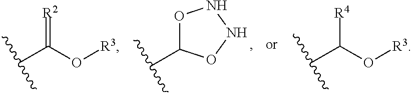

R² is oxygen, C, ═CₘNH₂, ═CₘOH, ═NCₘNH₂, or ═NCₘOH.
R³ is —Cₙ, —CₘOH, or —CₘNH₂.
R⁴ is —CₘOH, —Cₘ═NH, or —CₘNH₂.
R⁵ is hydrogen, halogen, cyano, hydroxyl, amino, oxo, —CHO, —SO₂, C₃-C₆cycloalkyl, C₃-C₅heterocycloalkyl, and C₁-C₆alkyl in which one carbon atom may be replaced by O, S, or NR⁷ and which C₁-C₆alkyl is optionally substituted with one or more substituents independently chosen from halogen, hydroxyl, amino, oxo, and —COOH.

R⁶ is independently chosen at each occurrence from hydrogen, halogen, hydroxyl, C₁-C₆alkyl, C₁-C₆alkoxy, C₃-C₇cycloalkyl, C₃-C₇cycloalkoxy, —C(O)C₁-C₆alkyl, —C(O)C₃-C₇cycloalkyl, C₁-C₂haloalkyl, and C₁-C₂haloalkoxy.

R⁷ is independently chosen at each occurrence from hydrogen and C₁-C₆alkyl.

The disclosure includes compounds of Formula (I) and salt thereof in which Y is

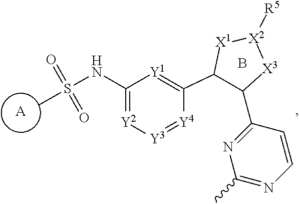

that is A is “Ring A.”

DABK is a compound having the structure

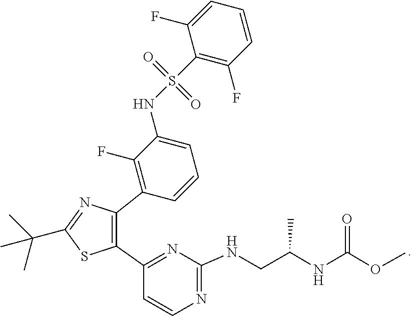 [SMILES: COC(=O)N[C@@H](C)CNc1nccc(-c2sc(C(C)(C)C)nc2-c2cccc(NS(=O)(=O)c3c(F)cccc3F)c2F)n1]

The disclosure includes pharmaceutical compositions comprising a compound of Formula I or salt thereof, together with a pharmaceutically acceptable carrier.

The disclosure includes methods of using a compound of Formula I or salt thereof, for treating a patient suffering from cancer, comprising administering a therapeutically effective amount of the compound or salt of Formula Ito the patient. Cancers that can be treated using a compound of Formula I include cancers dependent on dimers of RAF family, including cancers dependent on wild type BRAF dimers, BRAF^{V600E} monomers, BRAF^{V600E} dimers, dimers of BRAF splice variants (including p61-BRAF) and BRAF fusions, and BRAF dimers belonging to Class II and Class III. Examples of such cancers can include pediatric-low grade gliomas, BRAF^{V600E} gliomas, central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system). The cancer can be melanoma, thyroid cancer, hairy cell leukemia, ovarian cancer, lung cancer, pancreatic, prostate, gastric, endometrial or colorectal cancer. The cancer can be a cancer susceptible to treatment with a RAF dimer inhibitor.

The disclosure includes a method of treating a patient suffering from a cancer, comprising (a) determining that a cell of the cancer contains a BRAF^{V600E} mutation, and (b) administering a therapeutically effective amount of a compound of Formula I or salt thereof, to the patient.

### BRIEF DESCRIPTION OF THE DRAWINGS

FIG. 1. Comparison of inhibitory activity and binding affinity to BRAF^{V600E} and BRAF^{WT} of Formula I compound, DABK, and three clinically approved BRAF inhibitors, dabrafenib (DAB), vemurafenib (VEM), and encorafenib (Enco). Kinase inhibition data were produced with ZLYTE (Invitrogenn). Binding affinities were obtained using LanthaScreen (Initrogen).

FIG. 2. Inhibition of pERK in melanoma A375 cells by DABK, dabrafenib (DAB), and encorafenib (Enco) after treatment for 1 hour. pERK levels were derived densitometrically and were normalized to cells treated with vehicle (DMSO). Data shown are mean±SEM (n=3).

FIG. 3. In vitro residence times of VEM (FIG. 3A), DAB and Enco (FIG. 3B), and DABK (FIG. 3C) on BRAF^{V600E} Half-life (t1/2) of inhibitors on full length BRAF^{V600E} where obtained from exponential fits of inhibitor release profiles, upon addition of excess ATP-site tracer T178 (Invitrogen). Inhibitor release was detected as increase of TR-FRET signal, using LanthaScreen-Eu (Invitrogen). DMSO was used as control.

FIG. 4. Cellular engagement of DAB and DABK on BRAF^{V600E} by CETSA. CETSA analysis of DAB (FIG. 4A) and DABK (FIG. 4B) at 1, 5 and 20 μM in A375 melanoma cells. Normalized band intensities were obtained by densitometric analysis of Western Blots using Antibodies against BRAF, normalized to loading control (GAPDH). Treatment with DMSO was used to obtain control melting temperature (Tm). Reported Tm values were derived by least square fits of shown CETSA curves.

FIG. 5. Cellular recovery of ERK signaling by DABK vs. DAB. Wash-out experiments were conducted in A375 cells. After treatment with 500 nM inhibitors for 1 h (on time), cells were incubated with fresh medium for the indicated times (off time), followed by Western Blot analysis for p-MEK (FIG. 5A). FIG. 5B shows the half-life of DABK vs. DAB. DABK exhibited a half-life of 2.5 hours, while DAB exhibited a half-life of 34.3 minutes. Retention of p-MEK inhibition by DABK lasts approximately 4.4 times longer than MEK inhibition by DAB.

FIG. 6. DABK has a potent antiproliferative effect in BRAF-dependent tumor cell lines that are resistant to DAB. Dose dependent viability curves using ATP-Glo kit (Promega) were obtained upon treatment of cells with inhibitors for 72 hr. A375 (FIG. 3A): non-resistant BRAF^{V600E} melanoma, SK-MEL-239-C4 (FIG. 3B): resistant melanoma cells with p61-BRAF^{V600E} splice variant, RKO: resistant colorectal cells with BRAF^{V600E} (FIG. 3C).

FIG. 7. DABK demonstrates strong synergy with MEKi at lower concentrations than clinical inhibitors DAB and Enco, in KRASG12S lung adenocarcinoma cells (Cellosaurus A549). Extent of synergy in antiproliferative effect of combination treatment of BRAF inhibitors Enco (FIG. 7A), DAB (FIG. 7B), and DABK (FIG. 7C), DAB with MEK inhibitor cobimetinib (COB) was assessed using the Bliss matrix method. Cell viability curves at different COB concentrations are shown.

FIG. 8. KinomeScan of DAB (FIG. 8A) and DABK (FIG. 8B). DABK demonstrates higher specificity than DAB in a panel of 97 kinases.

FIG. 9. PD/PK study of DABK at 5 mg/kg. Mouse plasma levels were determined at 0.5, 1, 2, 4, 6, 12 and 24 hr after oral gavage of DABK and DAB suspended in vehicle. PD study was performed at 2, 12 and 24 hr post PO of inhibitors (3 mice per group), on tumors with max dimensions 100-150 mm³. Xenograft tumors were grown from A375 melanoma cells.

FIG. 10. Comparison of inhibitory activity of Formula I compounds to BRAFV600E, BRAFWT and CRAF (Y340D/Y341D). Kinase inhibition data were produced with ZLYTE (Invitrogen). IC50 values (in nM) were obtained by nonlinear regression fits. Plots are provided for compound K5 (FIG. 10A), K6 (FIG. 10B), K7 (FIG. 10C), K8 (FIG. 10D), K9 (FIG. 10E), and K10 (FIG. 10F).

FIG. 11. Antiproliferative effect of Formula I compounds in A375 tumor cell line. Dose dependent viability curves using ATP-Glo kit (Promega) were obtained upon treatment of cells with inhibitors for 72 hr. IC50 values (in nM) were obtained by nonlinear regression fits.

FIG. 12. Antiproliferative effect of Formula I compounds in SKMEL239-C4 tumor cell line. Dose dependent viability curves using ATP-Glo kit (Promega) were obtained upon treatment of cells with inhibitors for 72 hr. IC50 values (in nM) were obtained by nonlinear regression fits.

FIG. 13. Inhibition of ERK signaling in melanoma A375 cells by Formula I compounds after treatment for 1 hour. pER^{1/2} levels were derived densitometrically and were normalized to cells treated with vehicle (DMSO). Representative data are shown. IC50 values (in nM) were obtained by nonlinear regression fits. Plots are provided for compound K5 (FIG. 13A), K6 (FIG. 13B), K7 (FIG. 13C), K8 (FIG. 13D), and K9 (FIG. 13E).

FIG. 14. Recovery of MAPK signaling activity after washout in A375 cells. Cells were treated for 1 hr with 500 nM DABK (FIG. 14A), K6 (FIG. 14B) or K8 (FIG. 14C) (0 min time point), followed by washout with fresh media for indicated times and WB analysis. Total ERK1/2 was used as loading control. (lower) Relative p-MEK1/2 data obtained by densitometric analysis and normalized to loading control and untreated cells (DMSO) are plotted, indicating prolonged signaling inhibition over time.

FIG. 15. Recovery of MAPK signaling activity after washout in SKMEL239-C4 cells. Cells were treated for 1 hr with 500 nM DABK (FIG. 15A), K6 (FIG. 15B), or K8 (FIG. 15C) (0 min time point), followed by washout with fresh media for indicated times and WB analysis. Total ERK1/2 was used as loading control. (lower) Relative p-ERK1/2 data obtained by densitometric analysis and normalized to loading control and untreated cells (DMSO) are plotted, indicating prolonged ERK signaling inhibition over time. Almost complete p-MEK1/2 inhibition over time is also observed (middle panels).

### DETAILED DESCRIPTION

### Terminology

In order for the present disclosure to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that includes more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” or the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. The open-end phrases such as “comprising” include and encompass the close-ended phrases. Comprising may be amended to the more limiting phrases “consisting essentially of” of “consisting of” as needed.

The definition of each expression, e.g., alkyl, Y, Z, or the like, when it occurs more than once in any structure, is intended to be independent of its definition elsewhere in the same structure.

A dash (“—”) or a double bond symbol (“═”) that is not between two letters or symbols is used to indicate a point of attachment for a substituent.

A wavy line,

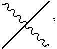

indicates a point of attachment of the substituent to the main structure.

Compounds of Formula I include compounds of the formula having isotopic substitutions at any position. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Compounds of Formula I also require enrichment of deuteration (substitution of a hydrogen atom with deuterium) at identified positions.

The term “alkyl” means a branched or unbranched aliphatic radical containing the indicated number of carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl. When —C₀-Cₙ alkyl is used in conjunction with another substituent, such as C₃-C₆cycloalkyl(C₀-C₂alkyl)- the other substituent group is bound to the group it substitutes by a single bond (C₀) or by an alkylene linker having the indicated number of carbon atoms.

An “alkylene” group is a bivalent saturated alkyl radical having the indicated number of carbon atoms.

The term “alkenyl” means a branched or unbranched hydrocarbon radical containing the indicated number of carbon atoms and having at least on carbon-carbon double bond. Representative examples of alkenyl include, but are not limited to, ethenyl, propenyl, butenyl, buta-1,3-dienyl, and the like.

“Alkoxy” is an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkenylene” means a bivalent hydrocarbon radical containing at least one carbon-carbon double bond and having the indicated number of carbon atoms.

“RAF kinase family” refers to RAF kinases including ARAF, BRAF and CRAF.

“Cyclolalkyl” is a saturated carbocyclic ring having the indicated number of carbon ring atoms, such as 3, 4, 5, 6, or 7 ring atoms, for example C₃-C₆)cycloalkyl is a cyclopropyl, cyclobutyl, cyclopentyl, or cyclohexyl group. “Cycloalkoxy” is a cycloalkyl group attached to the group it substitutes via an oxygen (—O—) linker.

“Haloalkyl” is an alkyl group as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

“Haloalkoxy” is a haloalkyl group as defined herein appended to the group it substitutes through an oxygen atom.

A “heterocycle” is a cyclic group containing at least on ring heteroatom chosen from N, O, and S. The heterocycle can be fully saturated, i.e. a heterocycloalkyl group, partially unsaturated, e.g. a heterocycloalkenyl group, or aromatic, e.g. a heteroaryl group. The heterocycle can contain one ring having 4 to 7 ring members and one, two, three, or four heteroatoms independently chosen from N, O, and S. It is preferred that not more than two heteroatoms are O or S and O and S atoms are not adjacent. The heterocyclic group can also contain two fused ring or two rings in spiro orientation; only one ring in a two ring heterocyclic group is required to contain a heteroatom.

“Heterocycloalkyl,” is saturated ring group, having the stated number of ring atoms, for example, 3-to 6-ring atoms or 3- to 5-ring atoms. 1 or 2 ring atoms are independently chosen from N, O, and S. Examples of heterocycloalkyl groups includes azepines, azetidinyl, morpholinyl, pyranyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

As used herein, the term “administering” means providing a pharmaceutical agent or composition to a subject, and includes, but is not limited to, administering by a medical professional and self-administering.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

“Pharmaceutical compositions” means compositions comprising at least one active agent, such as a compound or salt of Formula (I), and at least one other substance, such as a carrier. Pharmaceutical compositions meet the U.S. FDA's GMP (good manufacturing practice) standards for human or non-human drugs.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002.

As used herein, the term “patient” means a human or non-human animal, e.g. a companion animal such as a cat or dog, selected for treatment or therapy.

As used throughout this application, the term “pharmaceutically effective amount of a compound for pharmaceutical use” shall mean an amount of compound that exhibits the intended pharmaceutical or therapeutic or diagnostic effect when administered.

Suitable groups that may be present on a “substituted” or “optionally substituted” position include, but are not limited to, e.g., halogen; cyano; —OH; oxo; —NH₂; nitro; azido; alkanoyl (such as a C₂-C₆ alkanoyl group); C(O)NH₂; alkyl groups (including cycloalkyl and (cycloalkyl)alkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; mono- or dialkylamino groups including groups having alkyl groups from 1 to about 6 carbon atoms; mono- or dialkylcarboxamido groups (i.e. alkylNHC(O)—, (alkyl₁)(alkyl₂)NC(O)—, alkylC(O)NH—, or alkyl₁C(O)N(alkyl₂)-) having alkyl groups from about 1 to about 6 carbon atoms; carbocyclyl such as aryl having 6 or more carbons and one or more rings, (e.g., phenyl, biphenyl, naphthyl, or the like, each ring either substituted or unsubstituted aromatic); or a saturated, unsaturated, or aromatic heterocycle having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocycles may be further substituted, e.g. with hydroxy, alkyl, alkoxy, halogen and amino. In certain embodiments “optionally substituted” includes one or more substituents independently chosen from halogen, hydroxyl, oxo, amino, cyano, —CHO, —CO₂H, —C(O)NH₂, C₁-C₆-alkyl, C₂-C₆-alkenyl, C₁-C₆-alkoxy, C₂-C₆-alkanoyl, C₁-C₆-alkylester, (mono- and di-C₁-C₆-alkylamino)C₀-C₂-alkyl, (mono- and di-C₁-C₆-alkylamino)(CO)C₀-C₂-alkyl, C₁-C₂-haloalkyl, C₁-C₂haloalkoxy, and heterocyclic substituents of 5-6 members and 1 to 3 N, O or S atoms, i.e. pyridyl, pyrazinyl, pyrimidinyl, furanyl, pyrrolyl, thienyl, thiazolyl, triazinyl, oxazolyl, isoxazolyl, imidazolyl, tetrahydrofuranyl, tetrahydropyranyl, piperidinyl, morpholinyl, piperazinyl, and pyrrolidinyl, each of which heterocycle can be substituted by amino, C₁-C₆-alkyl, C₁-C₆-alkoxy, or —CONH₂. In certain embodiments “optionally substituted” includes halogen, hydroxyl, cyano, nitro, oxo, —CONH₂, amino, ono- or di-C₁-C₄alkylcarboxamide, and C₁-C₆hydrocarbyl , which C₁-C₆hydrocarbyl group, a hydrocarbon chain in which carbon atoms are joined by single, double or triple bonds, and any one carbon atom can be replaced by O, NH, or N(C₁-C₄alkyl) and which hydrocarbyl group is optionally substituted with one or more substituents independently chosen from hydroxyl, halogen, and amino. When the substituent is oxo (═O) then 2 hydrogen atoms are replaced. When an oxo group substitutes an aryl or heteroaryl group, aromaticity of the group is lost. When an oxo group substitutes a heteroaryl group the resulting heterocyclic group can sometimes have tautomeric forms. For example a pyridyl group substituted by oxo at the 2- or 4-position can sometimes be written as a hydroxypyridine. With regard to Group A in Formula I, “optionally substituted” can mean substituted with 0 or 1 or more substituents independently chosen from halo, hydroxyl, amino, cyano, C₁-C₆ alkyl, C₁-C₆alkoxy, C₁-C₂haloalkyl, and C₁-C₂haloalkoxy.

“Therapeutically effective amount” or “effective amount” refers to the amount of a compound that, when administered to a subject for treating or diagnosing or monitoring a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. In the context of cancer, treatment includes an amount sufficient to effect remission, an amount effect to shrink a tumor, an amount effective to halt or slow tumor growth, an amount effective to decrease the probability of developing cancer in a patient having a known risk factor for cancer, such as a mutation associated with the risk of developing cancer.

### Chemical Description

Compounds of Formula I may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

The term “chiral” refers to molecules, which have the property of non-superimpos ability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e. , they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

### RAF Inhibitors

The disclosure provides compounds of Formula I, as described in the SUMMARY section. Without wishing to be bound to any particular theory it is believed that these compounds exert anti-cancer activity by binding to and inhibiting RAF, including mutant BRAF forms, such as BRAF^{V600E}, found in many cancers.

In addition to compounds of Formula I the disclosure includes compounds and the salt thereof of the following subformulae of Formula I. The variables, e.g. R¹-R⁴, Y, and Z, carry the definitions set forth in the SUMMARY section unless otherwise specified. Any combinations of variable definitions is included in the scope of the disclosure so long as a stable compound results.

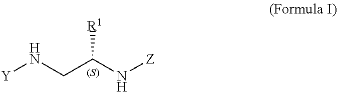

### The Y Tail

The disclosure includes compounds and salts of Formula I in which the following conditions are met for Ring A and Y¹-Y⁴.

The Ring A is a phenyl which is unsubstituted or substituted with one or more substituents independently chosen from halogen, hydroxyl, cyano, amino, C₃-C₆cycloalkyl, and C₁-C₆alkyl C₆alkyl in which one carbon atom may be replaced by O, S, or NR⁷ and which C₁-C₆alkyl is optionally substituted with one or more substituents independently chosen from halogen, hydroxyl, amino, oxo, and −COOH; and Y¹, Y², Y³, and Y⁴ are all CR⁶.

The Ring A is phenyl which is substituted with one or more halogen substituents; and Y¹, Y², Y³, and Y⁴ are all CR⁶ and R⁶ is independently chosen at each occurrence from hydrogen and halogen.

The disclosure includes compounds and salts of Formula I in which Y¹, Y², Y³, and Y⁴ are all CR⁶ and also includes compounds and salts of Formula I in which one of Y¹, Y², Y³, and Y⁴ is nitrogen.

The disclosure includes compounds and salts of Formula I in which wherein Y¹ is CR⁶ and R⁶ is F, Cl, Br, or methyl, and Y², Y³, and Y⁴ are CH.

The disclosure includes compounds and salts of Formula I in which the B ring is

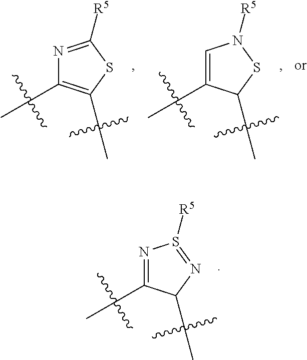

The disclosure includes compounds and salts of Formula I in which Y is

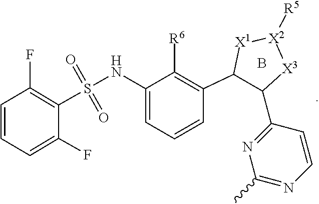

The disclosure includes compounds and salts of Formula I in which Y is

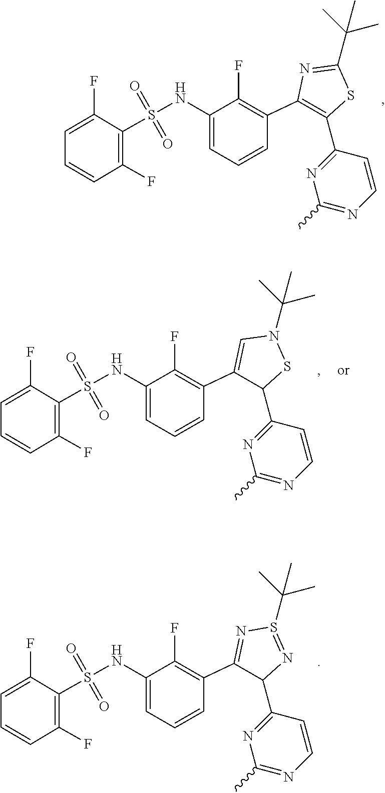

### The R¹ Variable

The disclosure includes compounds of Formula I having any of the above definitions for the Y tail , which R¹is methyl, ethyl, —CH₂NH₂, —CH₂CHNH, or —NHCH₃.

### The Z Variable

The disclosure includes compounds of Formula I having any of the above definitions in which Z carries one of the following definitions.

Z is

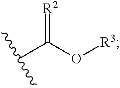

and R² is oxygen, ═CₙNH₂, ═CₙOH, ═NCₙNH₂, or ═NCₙOH, where n is 1, 2, 3, or 4. R³ is optionally —Cₙ, —CₙOH, or —CₙNH₂; where n is 1 or 2.

Z is

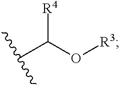

and R⁴ is —CₙOH, —Cₙ═NH, or —CₙNH₂; where n is 1, 2, 3, or 4. R³ is optionally —Cₙ, —CₙOH, or —CₙNH₂; where n is 1 or 2.

Z is

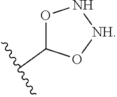 [SMILES: *C1ONNO1]

The disclosure includes compounds of Formula I, which have a range of Z values:

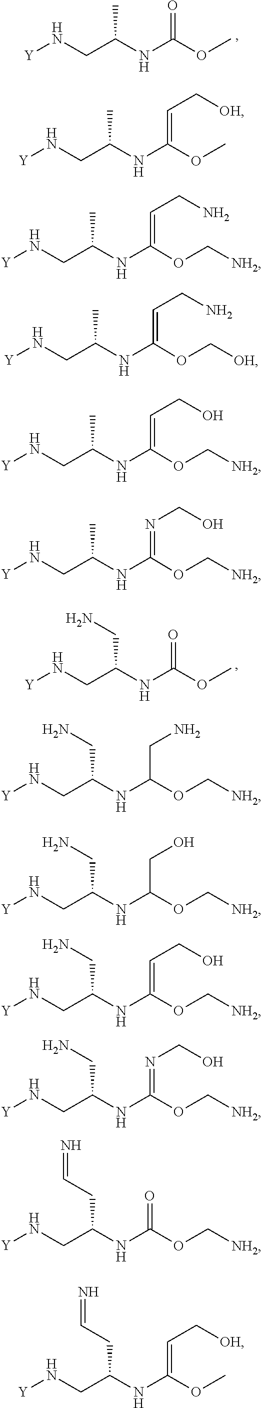

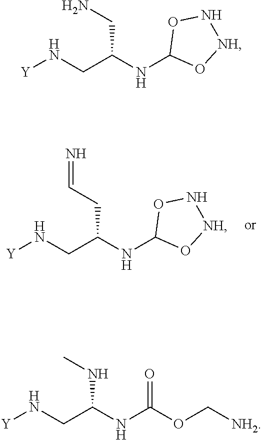

The disclosure includes the following compounds of Formula I and the salts thereof:

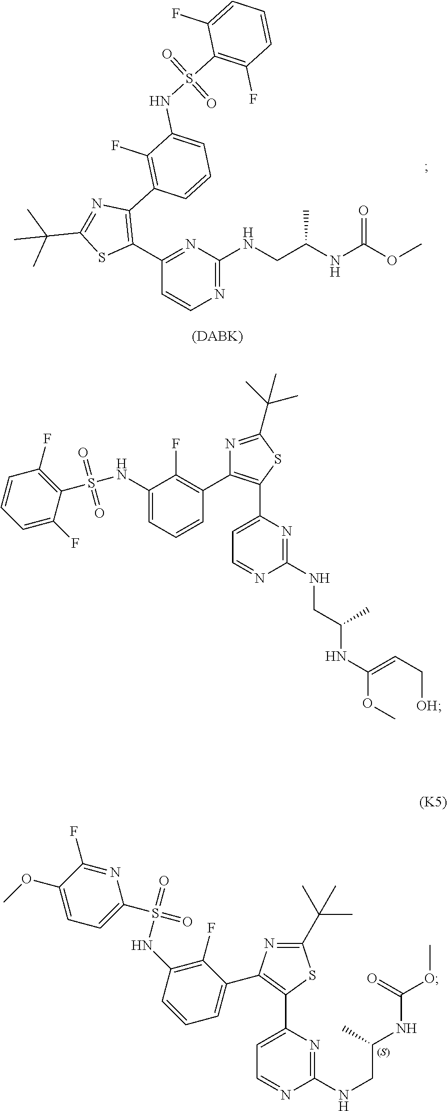

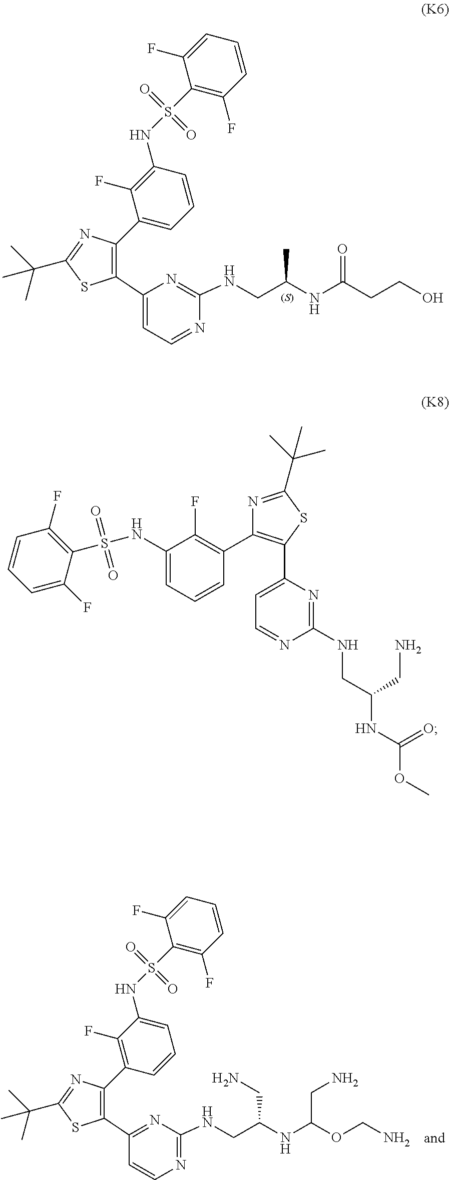

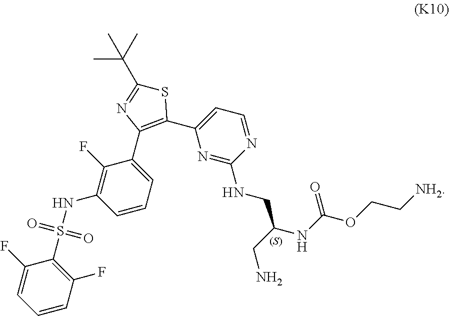

Additional compounds of the Formula (I) include compounds in which the “B ring” is dihydroisothiazol-5y1 group or a 2,5-thiadiazol-3-yl group. For example the disclosure includes the following compounds of Formula (I) and the salts thereof.

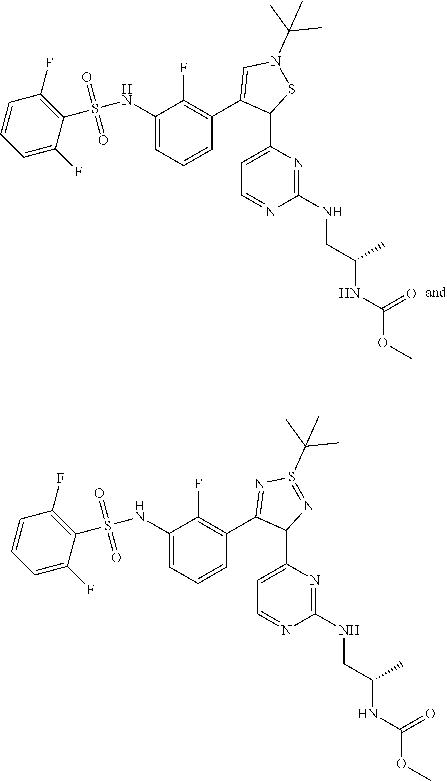 [SMILES: COC(=O)N[C@@H](C)CNc1nccc(C2SN(C(C)(C)C)C=C2c2cccc(NS(=O)(=O)c3c(F)cccc3F)c2F)n1 ; COC(=O)N[C@@H](C)CNc1nccc(C2N=S(C(C)(C)C)N=C2c2cccc(NS(=O)(=O)c3c(F)cccc3F)c2F)n1]

The disclosure includes compounds in which the “Y ring,” i.e. the ring containing Y¹-Y⁴, is pyridyl. For example the disclosure includes the following compounds and salts thereof.

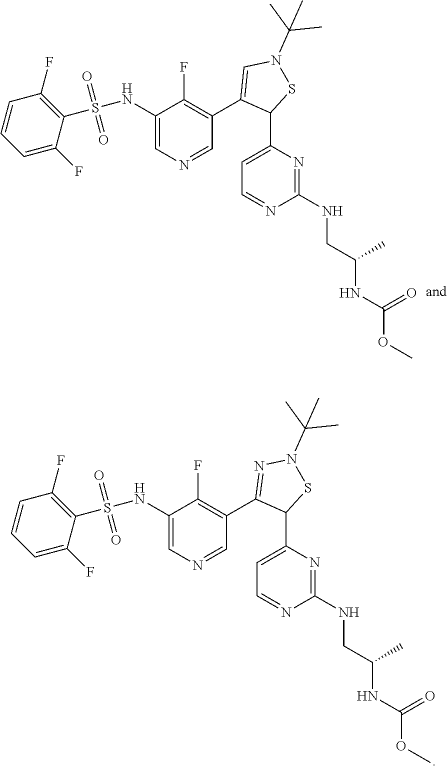 [SMILES: COC(=O)N[C@@H](C)CNc1nccc(C2SN(C(C)(C)C)C=C2c2cncc(NS(=O)(=O)c3c(F)cccc3F)c2F)n1 ; COC(=O)N[C@@H](C)CNc1nccc(C2SN(C(C)(C)C)N=C2c2cncc(NS(=O)(=O)c3c(F)cccc3F)c2F)n1]

The A group can be varied. For example, the A group can be a substituted by pyridyl, such as a 6-fluoro-5-methoxyp-pyrid-2-yl group, a di-alkylamino group, a 1-pyrrolyl group or a 1-piperazinyl group. The disclosure includes the following compounds and their pharmaceutically acceptable salts

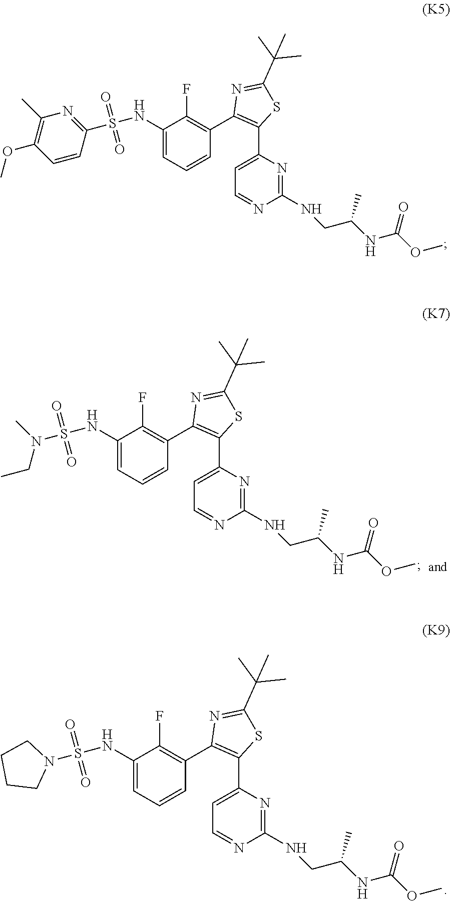

This disclosure provides RAF inhibitors, in which certain chemical moieties, comprising all portions of Formula I except the Y group are joined synthetically to kinase inhibitor scaffolds (the Y group) creating RAF inhibitors with increased kinetic selectivity (enzyme residence time), inhibition efficacy and target specificity, and at the same time conferring desirable pharmacokinetic properties.

There is a need for RAF inhibitors, including BRAF^{V600E} inhibitors, that have kinetic selectivity (increased enzyme residency times) combined with good bioavailability, factors known to determine the success of kinase inhibitors in the clinic. This disclosure provides certain compounds of Formula I that exhibit the desired bioavailability and increased kinase residence time.

Compounds of Formula I, include DABK. DABK may be understood as a combination of two components—DAB, which is a free radical of dabrafenib and occupies the Y position in Formula I and the K-tail, which is the rest of DABK. The Y and K-tail regions in other compounds of Formula I are as defined herein. Applicants have obtained biochemical, cellular and pharmacological insights of DABK in comparison to Dabrafenib (DAB). Although DAB and DABK have a similar ATP-binding scaffold, the K-tail in DABK attributes greatly improved preclinical properties to this compound.

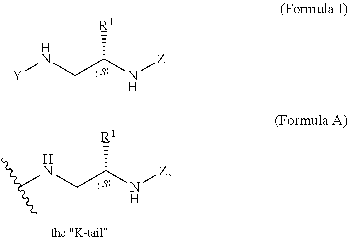

First, despite similar biochemical potencies (kinase activity, binding) between DABK and DAB, kinetic selectivity of DABK is greatly enhanced, as exemplified by the high on-target residence time (low K_{off} value) on BRAF^{V600E} in vitro and in cells.

Second, targeting selectivity of DABK across the kinome is improved compared to DAB's, as shown in competition screens (KinomeScan).

Third, cellular activity and specificity of DABK is greatly enhanced. For example, DABK inhibits the growth of resistant SK-MEL-239-C4 melanoma (IC50=35 nM) and colorectal RKO cells (IC50=20 nM), in comparison to IC50s>400 nM for DAB and other US FDA approved RAF inhibitors, which include Vem (vemurafenib) and Enco (encorafenib). In addition, DABK exerts strong synergy of inhibition at very low concentrations in resistant lung cancer cells (A549), in combination with the clinical MEK inhibitor, cobimetinib. To our knowledge, DABK is the first RAF inhibitor that demonstrates such remarkable inhibitory profiles in these highly resistant tumor cell lines.

Fourth, the PK/PD profile of DABK in a A375 (melanoma) mice xenograft tumor model is highly improved compared to DAB, with high endurance of pERK inhibition in tumors (>12 hr) despite low drug dose (5 mg/kg) and faster clearance from the blood. This corroborates the high cellular potency and on-target residence time found in biochemical kinase activity assay. To rationalize these results, we have determined the co-crystal structure of DABK and BRAF^{V600E} kinase, which shows the exact orientation of the K-tail. Comparison of the BRAF^{V600E}-DABK crystal structure with BRAF^{V600E}-DAB structure (not shown), reveals that the K-tail of Formula I compounds adopts conformationally distinct orientations within the BRAF site. Without wishing to be bound to any particular theory, we submit that the K-tail and its orientation of the K-tail on the RAF kinase binding site, including within RAF dimers produces the improved properties of DABK over DAB.

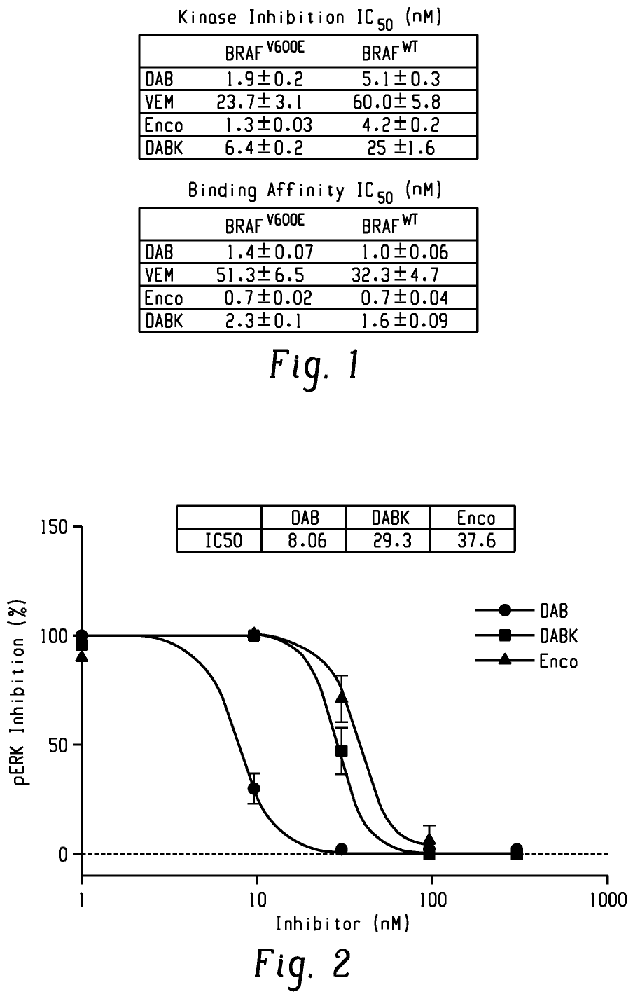

To assess the potency of DABK at equilibrium, we compared its inhibitory activity and binding affinity to BRAF^{V600E} and BRAF^{WT} with all three clinically approved BRAF inhibitors: dabrafenib (DAB), vemurafenib (VEM) and encorafenib (Enco). DABK exhibited an IC₅₀ and K_{d} in the low nM range, very similar to that of DAB. The IC₅₀ of DABK against BRAFWT was 5 times higher than the IC₅₀ of DAB against BRAFWT, supporting higher specificity for BRAF^{V600E} (FIG. 1). In cellular experiments monitoring pERK inhibition after inhibitor treatment of melanoma A375 cells expressing BRAF^{V600E} for 1 hr, DABK had comparable IC₅₀ to Enco, while DAB exhibited a lower IC₅₀ (FIG. 2). Enco demonstrates better residence time than DAB and VEM and sustainable anti-tumor effects in vivo, indicating that cellular equilibrium pERK half-inhibition values are not necessarily related to kinetic drug profiles and tumor inhibition end-effects in vivo.

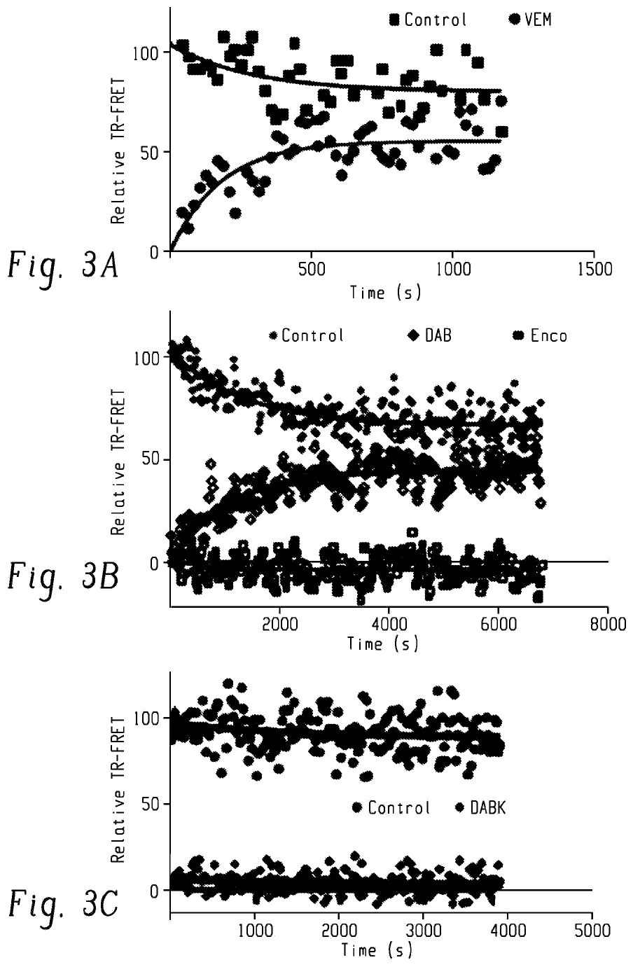

To evaluate the binding kinetics of these inhibitors to BRAF^{V600E}, we obtained apparent in vitro residence times (K_{off} values) using a kinase ATP-probe displacement assay. As expected by our chemical approach, DABK displays a K_{off} which is indicative of sustained binding in the time frame of the experiment, similarly to Enco, but in contrast to DAB and VEM, which have significantly shorter residence times (FIG. 3). Therefore, our chemical modification of DAB's ATP-binding scaffold modulates its kinetic properties and converts a clinical inhibitor with poor binding kinetics to one with highly favorable kinetics.

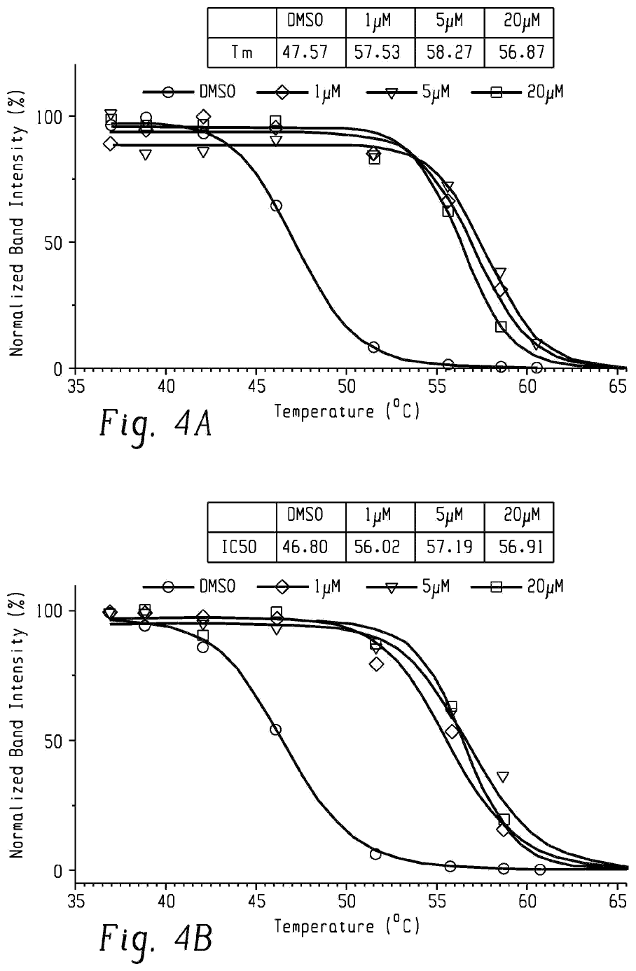

To validate these results are indeed a result of cellular engagement of DAB and DABK, we performed Cellular Thermal Shift Assay (CETSA) after treatment of A375 cells with increasing concentrations of DAB or DABK. The obtained melting temperature (Tₘ) values were very similar (FIG. 4), indicating that both inhibitors target BRAF^{V600E} in cells with similar efficiency.

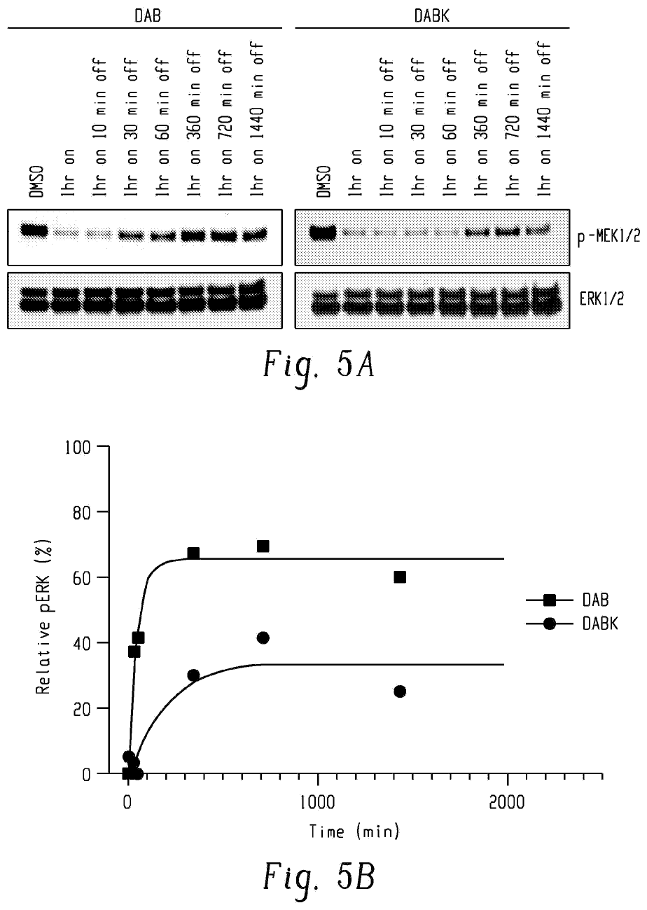

Subsequently, we set out to demonstrate that the biochemically determined lower K_{off} value of DABK compared to DAB also translates in slower recovery of ERK inhibition in A375 cells. We monitored recovery of pMEK in wash-out experiments, as this kinase is the direct cellular target of BRAF. We observed that under DAB treatment, recovery of pMEK inhibition has a half-life of about 34 min in contrast to a much longer recovery of 2.5 hr when cells are treated with DABK (FIG. 5). DABK exhibited a half-life of 2.5 hours, while DAB exhibited a half-life of 34.3 minutes. These data agree with our biochemical studies confirming that, compared to DAB, DABK has a much better kinetic selectivity for BRAF^{V600E} in cancer cells.

To rationalize structurally these observations, we determined the crystal structure of BRAF^{V600E} DAB and compared it to the structure of BRAF^{V600}-DABK (not shown). The structural analysis suggests that the core of DAB and DABK adopts an almost exactly same orientation in the ATP-binding pocket, the K-tail of DABK displays conformational variability in its binding of protomers in the BRAF dimer, with a closed conformation stabilized by a network of water mediated H-bonding interactions and an open conformation. We hypothesize that this entropic contribution of the K-tail is responsible for the enhanced residence time of DABK in its binding site.

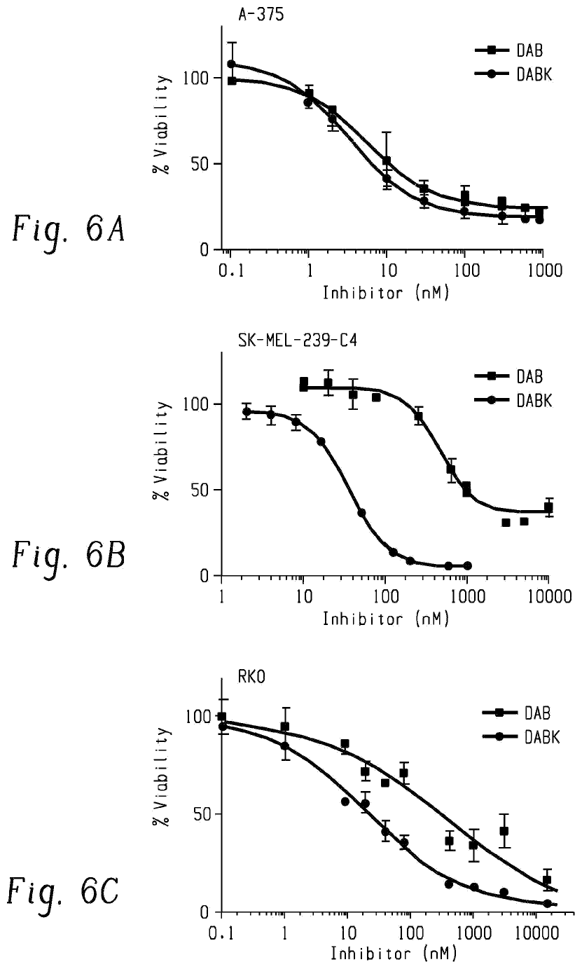

Remarkably, this enhanced kinetic specificity of DABK is translated into better antiproliferative effects in resistant melanoma and colorectal cell lines. As shown in FIG. 6, although DABK and DAB show similar ICsos for inhibiting cell growth in A375 cells (FIG. 6a), inhibition of viability by DABK is greatly enhanced in SK-MEL-239-C4 melanoma (FIG. 6b) or RKO colorectal cells (FIG. 6c), which are resistant to clinical RAF inhibitors. For example, DABK inhibits the growth of resistant SK-MEL-239-C4 melanoma (IC₅₀=35 nM) and colorectal RKO cells (IC₅₀=20 nM) with better potency by two orders of magnitude in comparison to DAB and all other known RAF inhibitors (IC₅₀_{^{S}}>400 nM). The IC₁₀_{^{S}} for cell viability are given in Table 1.

TABLE 1
Viability IC₅₀ (nM)

| | A375 | SK-MEL-239-C4 | RKO |
|---|---|---|---|
| DABK | 3.4 | 35.3 | 20.6 |
| DAB | 5.3 | 456 | 425 |

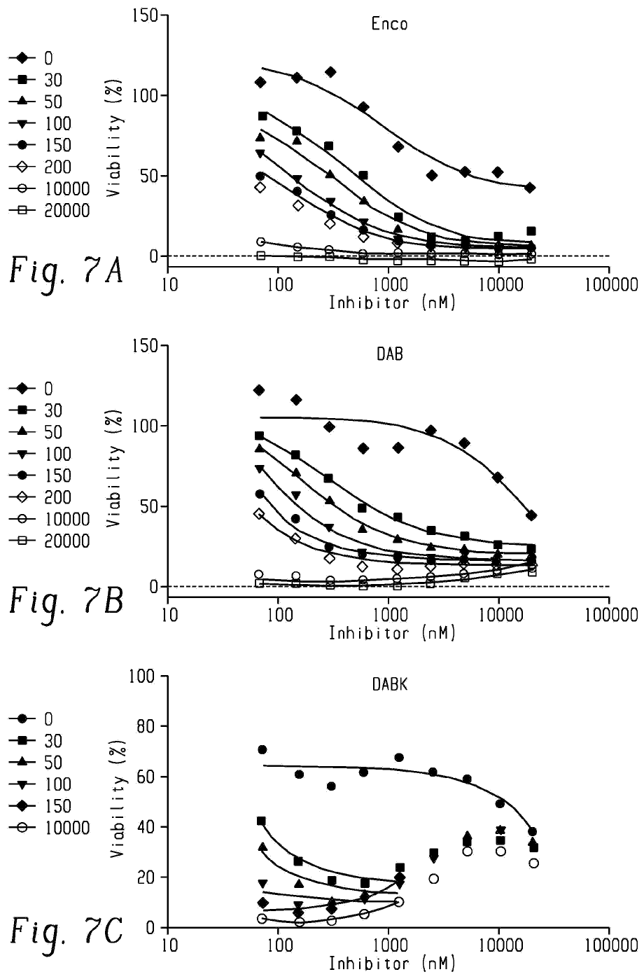

Most importantly, these results are extended into unprecedented synergy effects upon co-treatment with FDA approved MEK inhibitor cobimetinib (COB), in resistant lung cancer cells (A549), which bear KRAS^{G12S}/BRAF^{WT} mutation. Proliferation of these cells is usually inhibited by RAF inhibitors with IC₅₀ values of >1 μM. As shown in FIG. 7, co-treatment of these cells with Enco-COB (FIG. 7a) or DAB-COB (FIG. 7b) indeed results in very weak synergy or synergy peaking at >1 μM RAF inhibitor, respectively. In contrast, synergy from DABK-COB (FIG. 7c) co-treatment peaks at <300 nM. To our knowledge, DABK is the first RAF inhibitor that demonstrates such remarkable inhibitory profile in this highly resistant tumor cell line.

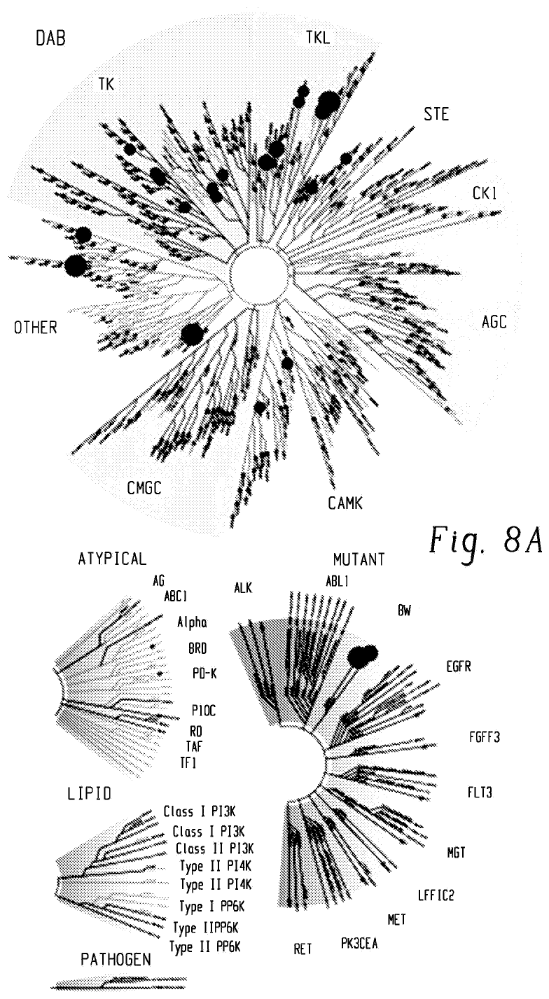

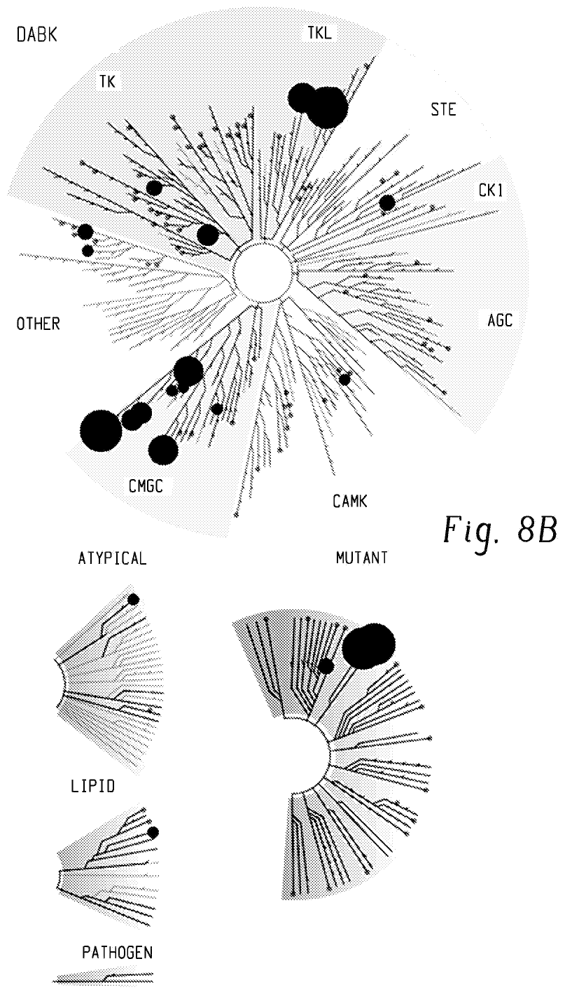

The superior kinetic selectivity of DABK compared to DAB is also results in improved targeting selectivity across the kinome, as shown by a KinomeScan assay FIG. 8.

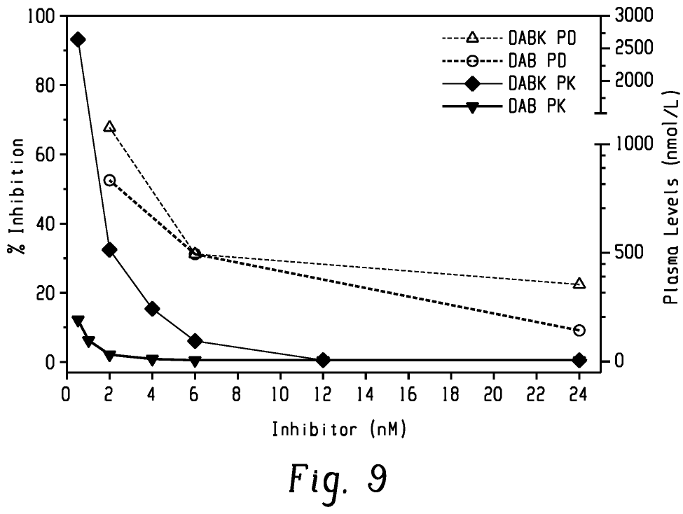

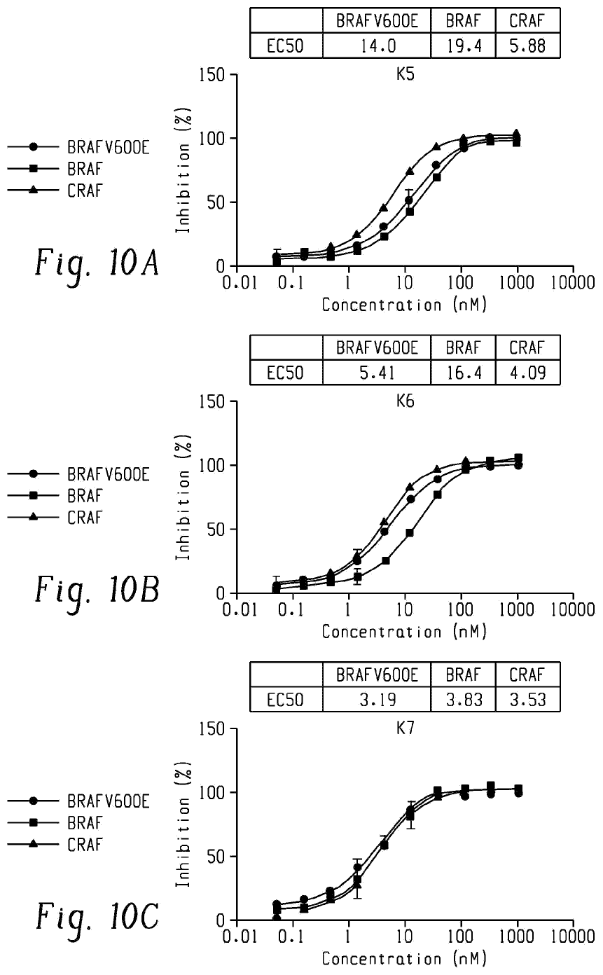

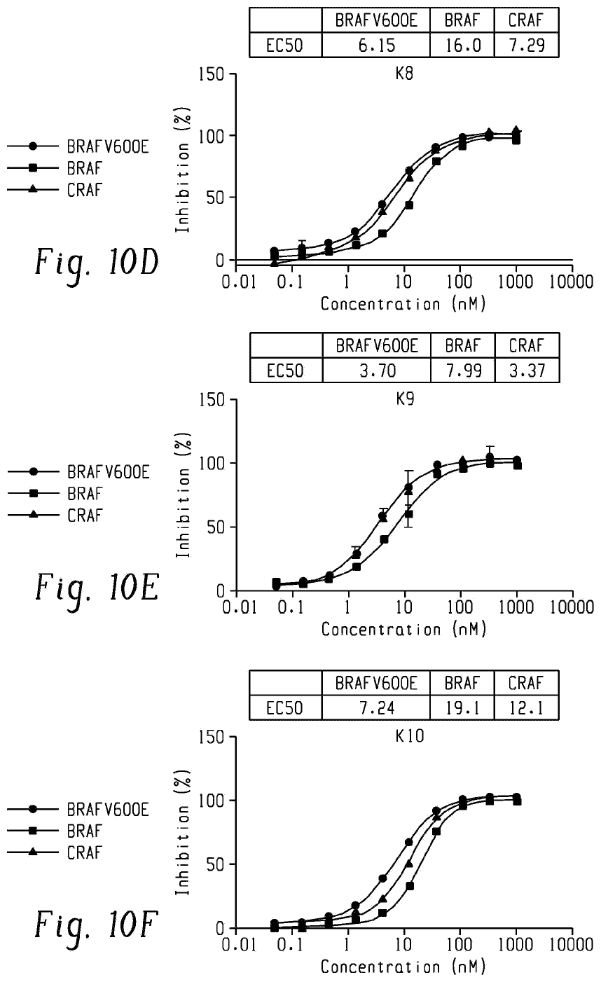

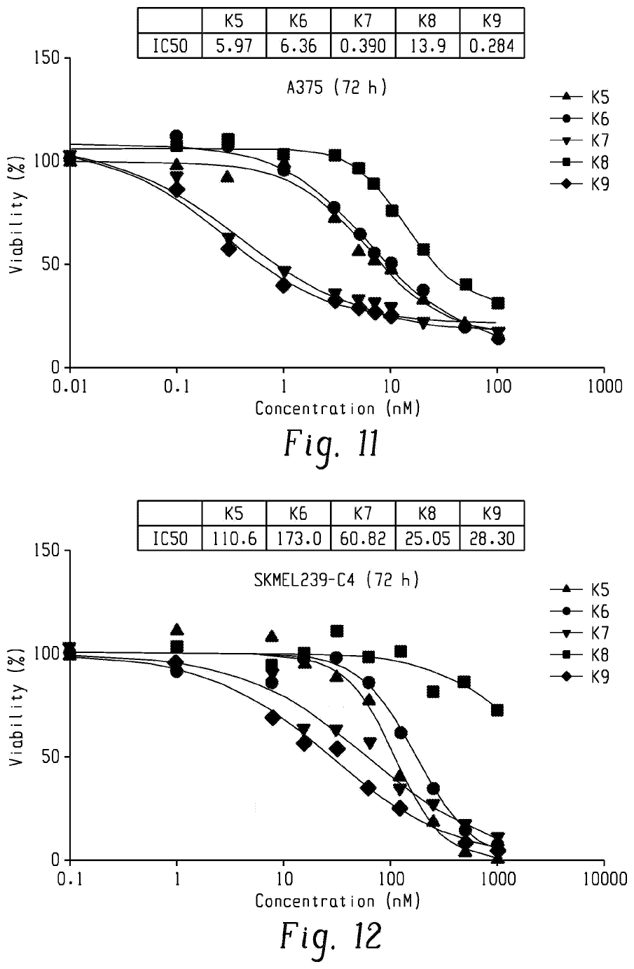

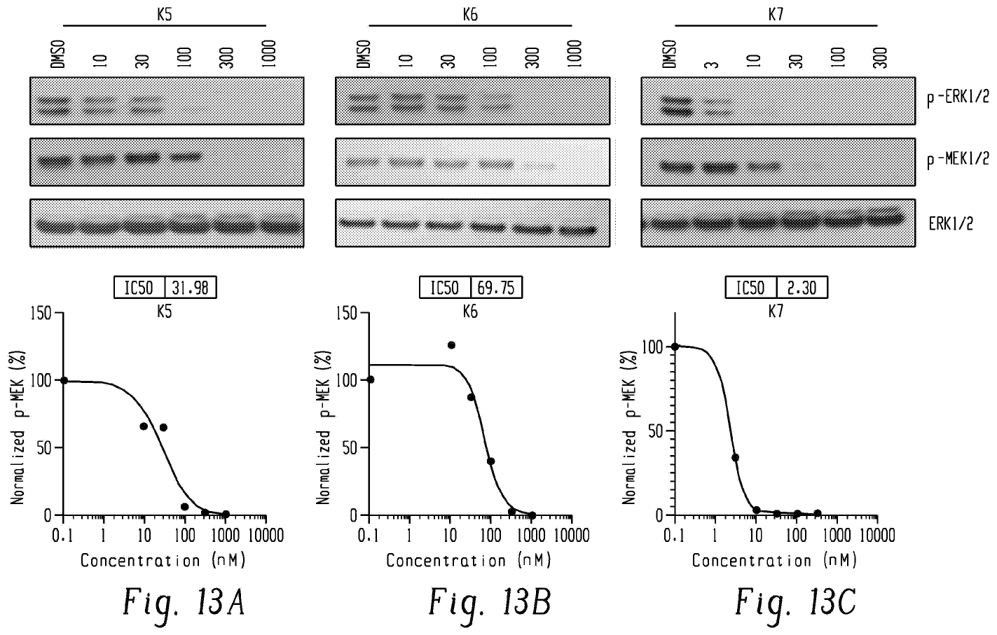

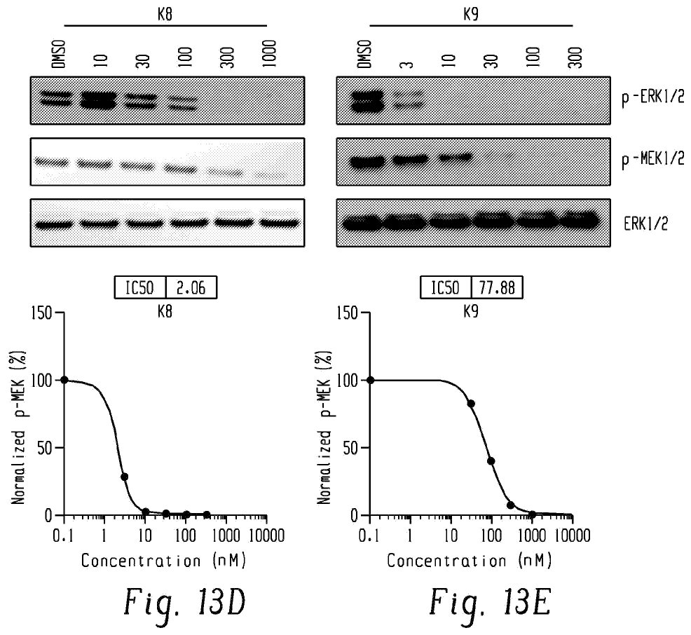

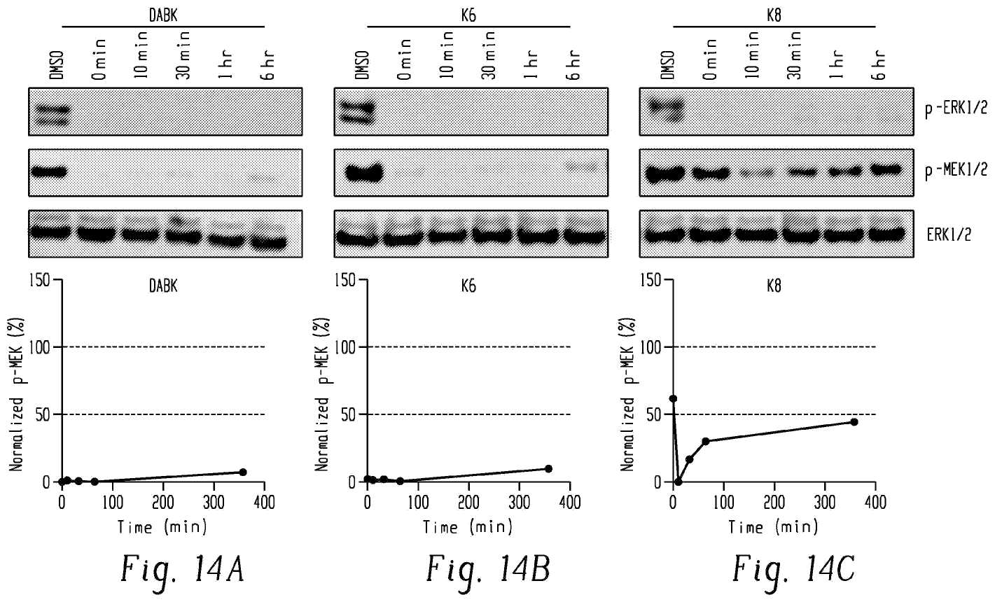

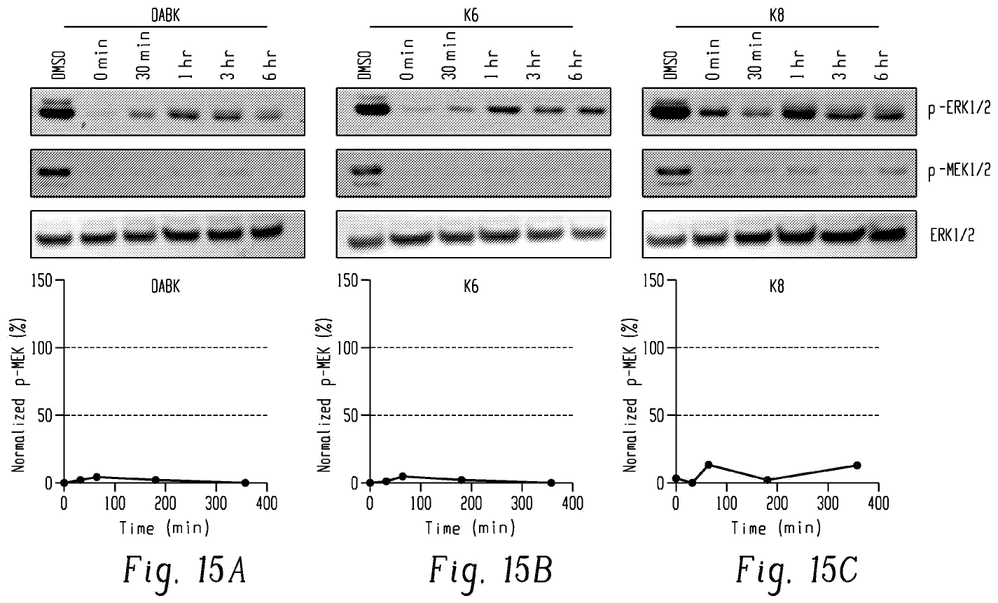

Finally, a low drug dose (5 mg/kg) of DABK shows faster clearance from the blood than DAB (FIG. 9), however the PK/PD profile of DABK in a A375 mice xenograft tumor model is highly improved with high endurance of pERK inhibition in tumors (>12 hr). These data suggest that increased target residence time in vivo can enable more efficacious tumor treatment with less dose and therefore increased therapeutic window.

Taken together, these results support the utility of compounds of Formula Ito favorably transform kinetic selectivity without loss of its binding potency, target specificity, anti-tumor growth activity and pharmacokinetic/pharmacodynamic properties of an FDA approved inhibitor.

Thus the disclosure provides unique compounds of Formula I with greatly improved preclinical and pharmacological properties over known RAF inhibitors.

### Pharmaceutical Compositions

The disclosure includes pharmaceutical compositions comprising a compound of Formula I or a salt thereof.

The disclosure includes methods in which one or more compounds are an admixture or otherwise combined with one or more compounds and may be in the presence or absence of commonly used excipients (or “pharmaceutically acceptable carriers”); for example, but not limited to: i) diluents and carriers such as starch, mannitol, lactose, dextrose, sucrose, sorbitol, cellulose, or the like; ii) binders such as starch paste, gelatin, magnesium aluminum silicate, methylcellulose, alginates, gelatin, sodium carboxymethyl-cellulose, polyvinylpyrrolidone or the like; iii) lubricants such as stearic acid, talcum, silica, polyethylene glycol, polypropylene glycol or the like; iv) absorbents, colorants, sweeteners or the like; v) disintegrates, (e.g., calcium carbonate and sodium bicarbonate) such as effervescent mixtures or the like; vi) excipients (e.g. cyclodextrins or the like); vii) surface active agents (e.g., cetyl alcohol, glycerol monostearate), adsorptive carriers (e.g., kaolin and bentonite), emulsifiers or the like. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, or the like, which are suitable for use in the compositions.

Furthermore, the disclosure includes compositions prepared using conventional mixing, granulating, or coating methods and may contain 0.01 to 90% of the active ingredients. In some embodiments, the one or more compounds are for pharmaceutical use or for diagnostic use. Such methods can be used, for example, to prepare a bio-enhanced pharmaceutical composition in which the solubility of the compound(s) is (are) enhanced. In some embodiments, the resulting compositions contain a pharmaceutically effective amount of a compound for pharmaceutical or diagnostic use. The resulting compositions (formulations) may be presented in unit dosage form and may be prepared by methods known in the art of pharmacy. All methodology includes the act of bringing the active ingredient(s) into association with the carrier which constitutes one or more ingredients. Therefore, compositions (formulations) are prepared by blending active ingredient(s) with a liquid carrier or a finely divided solid carrier, and/or both, and then, if needed, shaping the product into a desired formulation.

Typical compositions of the disclosure contain compound from about 90 to about 80% by weight, from about 80 to about 70% by weight, from about 70 to about 60% by weight, from about 60 to about 50% by weight, from about 50 to about 40% by weight, from about 40 to about 30% by weight, from about 30 to 20% by weight, from about 20 to about 10% by weight, from about 10 to about 4% by weight, from about 4.0% to about 2.0% by weight, from about 2.0% to about 1.0% by weight, and even from about 1.0% to about 0.01% by weight. The effective amount of compounds or compositions of the disclosure may range from about 0.1 to 100 milligrams (mg) per kilogram (kg) of subject weight. In certain embodiments, the compounds or compositions of the disclosure are administered at from about 0.0001 mg/kg to 0.1 mg/kg (e.g. diagnostic monitoring), or from 0.1 mg/kg to 2 mg/kg, or from about 2 mg/kg to 5 mg/kg; in other embodiments, from about 5 mg/kg to 10 mg/kg, from about 10 mg/kg to 20 mg/kg, from about 20 mg/kg to 30 mg/kg, from about 30 mg/kg to 40 mg/kg, from about 40 mg/kg to 50 mg/kg, from about 50 mg/kg to 75 mg/kg or from about 75 mg/kg to 100 mg/kg.

It should be understood that the ingredients particularly mentioned above are merely examples and that some embodiments of formulations comprising the compositions of the present disclosure include other suitable components and agents. The invention further includes packages, vessels, or any other type of container that contain a compound of the present invention.

### Methods of Treatment

The disclosure includes methods of treating a patient suffering from cancer, comprising administering a compound of Formula I or salt thereof to the patient. The cancer can be a cancer susceptible to treatment with a RAF inhibitor. Cancers dependent on RAF inhibition, include cancers dependent on wild type BRAF, BRAF^{V600E}, BRAF splice variants (including p61BRAF), mutant BRAF belonging to Class II and Class III and BRAF fusions. In certain embodiments the cancer is melanoma, thyroid, non-small cell lung cancer, colorectal, ovarian, pancreatic, prostate, gastric, endometrial, hairy cell leukemia, pediatric-low grade glioma, BRAF^{V600E} glioma, central nervous system tumor, such as a primary CNS tumors including glioblastomas, astrocytomas (e.g., glioblastoma multiforme) and ependymomas, or a secondary CNS tumors (i.e., metastases to the central nervous system of tumors originating outside of the central nervous system).

Other RAF dependent cancers, including Barret's adenocarcinoma, billiary tract carcinomas, breast cancer, cervical cancer, cholangiocarcinoma, large intestinal colon carcinoma, gastric cancer, carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia, hepatocellular carcinoma, endometrial cancer, pancreatic cancer, pituitary adenoma, prostate cancer, renal cancer, sarcoma, may also be treated by administering a compound of Formula I or salt thereof to a patient having such a cancer.

The cancer can be melanoma, colorectal cancer, hairy cell leukemia, ovarian cancer, lung cancer, or thyroid cancer. In certain embodiments the cancer in a cancer having a BRAF^{V600E} mutation.

The disclosure includes a method of treating a patient suffering from a cancer, comprising
(a) determining that a cell of the cancer contains a BRAF^{V600E} mutation, and
(b) administering a therapeutically effective amount of a compound of Formula I or salt thereof to the patient.

In some embodiments, the one or more compounds, or compositions of the present disclosure, are administered to persons or animals to provide substances in any dose range that will produce desired physiological or pharmacological results. Dosage will depend upon the substance or substances administered, the therapeutic endpoint desired, the diagnostic endpoint desired, the desired effective concentration at the site of action or in a body fluid, and the type of administration. In some embodiments, the compounds and compositions of the present disclosure may be administered to a subject. Suitable subjects include a cell, population of cells, tissue or organism. In certain embodiments, the subject is a mammal such as a human. The compounds may be administered in vitro or in vivo.

Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration, administration into the lumen or parenchyma of an organ, and parenteral administration. The compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art.

The compound of Formula I can be the only active agent administered to a patient or it can be administered together with another active agent. Other active agents that can be administered together with a compound of Formula I or salt thereof include MEK inhibitors such as trametinib, cobimetinib, binimetinib, or selumetinib, RAF inhibitors such as vemurafenib, sorafenib, encorafenib, and dabrafenib. Other active agents that can be administered together with a compound of Formula I or salt thereof include ERK inhibitors, RTK inhibitors, SHP2 inhibitors, KRAS mutant inhibitors, a RAF inhibitor, an MEK inhibitor, NRAS mutant inhibitors, CDK4/6 inhibitors, and PI3K inhibitors.

There are large numbers of antineoplastic agents available in clinical use, that may be used in combination with a compound of Formula I or a salt thereof. And there are several major categories of such antineoplastic agents, namely, antibiotic-type agents, alkylating agents, antimetabolite agents, hormonal agents, immunological agents, interferon-type agents, and a category of miscellaneous agents.

A first family of antineoplastic agents which may be used in combination with compounds of the present invention includes antimetabolite-type/thymidylate synthase inhibitor antineoplastic agents. Suitable antimetabolite antineoplastic agents may be selected from but not limited to the group consisting of 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(21-furanidyl) fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin.

A second family of antineoplastic agents which may be used in combination with compounds of the present invention consists of alkylating-type antineoplastic agents. Suitable alkylating-type antineoplastic agents may be selected from but not limited to the group consisting of aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, Erba distamycin derivatives, elmustine, estramustine phosphate sodium, fotemustine, hepsulfam, ifosfamide, iproplatin, lomustine, mafosfamide, mitolactolf, oxaliplatin, prednimustine, ranimustine, semustine, SmithKline spiromus-tine, tauromustine, temozolomide, teroxirone, tetraplatin and trimelamol.

A third family of antineoplastic agents which may be used in combination with compounds of the present invention consists of antibiotic-type antineoplastic agents. Suitaaclarubicin, actinomycin D, actinoplanone, aeroplysinin derivative, anthracycline, azino-mycin-A, bisucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-AI, esperamicin-Alb, fostriecin, glidobactin, gregatin-A, grincamycin, herbimycin, idarubicin, illudins, kazusamycin, kesarirhodins, menogaril, mitomycin, mitoxantrone, neoenactin, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, sorangicin-A, sparsomycin, terpentecin, thrazine, tricrozarin A, and zorubicin.

A fourth family of antineoplastic agents which may be used in combination with compounds of Formula I consists of a miscellaneous family of antineoplastic agents, including tubulin interacting agents, topoisomerase II inhibitors, topoisomerase I inhibitors and hormonal agents, selected from but not limited to the group consisting of x-carotene, X-difluoromethyl-arginine, acitretin, alstonine, amonafide, amphethinile, amsacrine, Angiostat, ankinomycin, anti-neoplaston A10, antineoplaston A2, antineoplaston A3, antineoplaston A5, antineoplaston aphidicolin glycinate, asparaginase, Avarol, baccharin, batracylin, benfluron, benzotript, bisantrene, bromofosfamide, caracemide, carmethizole hydrochloride, chlorsulfaquinoxalone, clanfenur, claviridenone, crisnatol, curaderm, cytochalasin B. cytarabine, cytocytin, DABIS maleate, dacarbazine, datelliptinium, didemnin-B, dihaematoporphyrin ether, dihydrolenperone, dinaline, distamycin, docetaxel elliprabin, elliptinium acetate, ergotamine, etoposide, etretinate, fenretinide, gallium nitrate, genkwadaphnin, grifolan NMF5N, hexadecylphosphocholine, homoharringtonine, hydroxyurea, ilmofosine, isoglutamine, isotretinoin, leukoregulin, lonidamine, marycin, merbarone, merocyanlne derivatives, methylanilinoacridine, minactivin, mitonafide, mitoquidone mopidamol, motretinide, N-acylated-dehydroalanines, nafazatrom, nocodazole derivative, Normosang (human hemin), ocreotide, oquizanocine, paclitaxel, pancratistatin, pazelliptine, ICRT peptide D, piroxantrone, polyhaematoporphyrin, polypreic acid, Efamol porphyrin, probimane, procarbazine, proglumide, razoxane, restrictin-P, retelliptine, retinoic acid, spatol, spirocyclopropane derivatives, spirogermanium, strypoldinone, Stypoldione, superoxide dismutase, teniposide, thaliblastine, tocotrienol, topotecan, Topostin, vinblastine sulfate, vincristine, vindesine, vinestramide, vinorelbine, vintriptol, vinzolidine, and withanolides. Alternatively, the present compounds may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ancestim, bexarotene, bicalutamide, broxuridine, capecitabine, celmoleukin, cetrorelix, cladribine, clotrimazole, cytarabine ocfosfate, daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfaconl, interferon alpha, natural, interferon beta, interferon beta-Ia, interferon beta-Ib, interferon gamma, natural interferon gamma-Ia, interferon gamma-Ib, interleukin-I beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; ambamustine, antisense oligonucleotide, bcl-2 (Genta), cetuximab, decitabine, dexaminoglutethimide, diaziquone, EL 532 (Elan), EM 800 (Endorecherche), eniluracil, etanidazole, fenretinidel filgrastim SDO1 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin iproxifene, LDI 200 (Milkhaus), leridistim, lintuzumab, idiotypic, polymorphic epithelial mucin-yttrium 90 MAb (Antisoma), marimastat, menogaril, mitumomab, motexafin, gadolinium, Galderma, nelarabine, nolatrexed, P 30 protein, pegvisomant, pemetrexed, porfiromycin, prinomastat, RL 0903 (Shire), rubitecan, satraplatin, sodium phenylacetate, sparfosic acid, etrathiomolybdate, thaliblastine, thrombopoietin, tin ethyl etiopurpurin, tirapazamine, cancer vaccine (Biomira), melanoma vaccine melanoma oncolysate vaccine (New York Medical College), viral melanoma cell lysates vaccine (Royal Newcastle Hospital), or valspodar.

### Biological Assays

BRAF kinase is a critical effector of the ERK signaling pathway, which is hyperactivated in many cancers. Oncogenic BRAF^{V600E} kinase signals as an active monomer in the absence of RAS-GTP, however, in many tumors BRAF dimers mediate ERK signaling. Although clinical RAF inhibitors effectively target BRAF^{V600E} monomers, prior to this disclosure selective inhibitors of BRAF dimers were elusive.

### EXAMPLES

### General Methods

### Antibodies

BRAF (Santa Cruz sc-5284), CRAF (Santa Cruz C-12) MEK1 (Millipore), MEK1/2 (Cell Signaling 4694), P-MEK1/2 (Cell Signaling 9154), ERK1/2 (Cell Signaling 4696), ERK1 (Santa Cruz sc-7383), P-ERK1/2 (Cell Signaling 4370), P-ERK1 (Santa Cruz 94), Actin (Invitrogen).

### Cell Culture

A375, SKMEL30 and SKMEL2 cells were grown in Dulbecco's modified Eagle's medium (DMEM) with 10% fetal bovine serum (PBS), 1% Pen-Strep, 1% Glutamine SKMEL239 C4 cells were grown in Dulbecco's modified Eagle's medium (DMEM) with 10% fetal bovine serum (FBS), 1% Pen-Strep, 1% Glutamine in the presence of 1 μM Vemurafenib. CALU6 cells were grown in Roswell Park Memorial Institute medium (RPMI) with 10% fetal bovine serum (PBS), 1% Pen-Strep, 1% Glutamine

### Western Blotting and Cellular ERK Signaling

Western blots were performed from whole cell lysates (WCLs) prepared in lysis buffer containing 50 mM Tris-HCl pH7.5, 1% NP40, 150 mM NaCl, 1 mM EDTA and 10% glycerol in the presence of protease inhibitor cocktail (Roche). WCLs were separated on a 4-12% NuPAGE MES gel (Invitrogen), transferred onto a PVDF membrane, blocked for lhr and immunoblotted with the corresponding antibodies.

### Wash Out and Cellular Recovery of MAPK Signaling

Wash-out experiments were conducted in A375 cells. After treatment with inhibitors at 500 nM for 1 h (on time), cells were incubated with fresh medium for the indicated times (off time). p-MEK levels were determined by WB and were quantified by densitometric analysis. p-MEK levels were normalized to total ERK1/2 which was used as loading control and control cells (DMSO treatment). Data were fit to an exponential model using least squares, to obtain apparent half-life (t_{1/2}) values.

### Densitometric Analysis and Quantification

Densitometric data for p-ERK, p-MEK, ERK1/2 (MAPK cellular activity) or BRAF and GAPDH (CETSA analysis) from western blot scanned films were obtained using Image Studio software (LI-COR). Data were corrected to loading control (total ERK1/2 or GAPDH) and normalized to DMSO treated bands (100%) and blot backgrounds (0%). IC50 or Tm values were obtained from non-linear regression fits of normalized data to a four-parameter logistic curve (4PL).

### Cell Viability Assay and Antiproliferative Synergy

For cell viability and proliferation assays, we followed the manufacturer's protocol for Cell-Titer Glo (Promega). In brief, cells were plated in 96-well plates at a density of 5000 cells per well. The next day, cells were treated with increasing concentrations of inhibitors for 72 hours at 37° C. At the end of the incubation period, 100 μL of Cell-Titer Glo (Promega) was added to each well and further incubated for 15 min at room temperature. Cell viability was determined by measuring luminescence and was detected by a F200 PRO microplate reader (TECAN). Viability assays were performed in at least triplicate and the data normalized to vehicle-treated control wells. IC₅₀ values were determined by nonlinear regression analysis using Prism software (Graphpad). Antiproliferative synergy was determined by co-treatment of inhibitors at indicated concentrations in 96-well plated at a density of 3000 cells per well. Inhibitors or DMSO control were injected using a D300e digital dispenser (TECAN). Extend of synergy was quantified using the BLISS matrix method.

### Cloning, Expression and Purification of BRAF

Human BRAF kinase domain (residues 443-723) with V600E mutation in addition to designed mutations to improve expression in E. coli as previously described¹² was cloned into the first multiple cloning site of a pET-28a vector, which expresses a hexa-histidine tag at the N-terminus of BRAF. Recombinant protein was transformed and expressed into E. coli strain BL21-Codon Plus(DE3)-RIPL (Agilent Technologies). Protein purification was performed by a rapid two-step procedure using nickel-affinity chromatography (Ni-NTA) followed by size exclusion chromatography with Superdex200HR 10/30 (GE Healthcare). Ponatinib or PHH at 1.5 molar excess to the protein sample was added immediately after elusion from Ni-NTA column.

### Kinase Activity Assay

BRAF kinase assays were performed using the Z′-LYTE™ enzymatic assay (Invitrogen, USA). Briefly, kinase activity was monitored in a cascade system consisting a mixture of inhibitor with BRAF or BRAF^{V600E}/inactive MAP2K1 (MEK1)/inactive MAPK1 (ERK2)/Ser/Thr 03 peptide (Invitrogen) in 50 mM HEPES pH 7.5, 100 μM ATP, 10 mM MgCl2, 1 mM EGTA, 0.01% Brij-35. Titrations were performed using a 1:3 dilution. Assays were performed using SelectScreen (Invitrogen).

### Binding Affinity

Binding affinity of inhibitors to recombinant full-length BRAF was determined using the LanthaScreen Eu Kinase Binding Assay (Invitrogen) in PBS buffer. Initially, saturated binding of fluorescent Alexa Fluor 647 ATP-site tracer T178 (Invitrogen) on BRAF, which was his-tagged at the N-terminus, was established. T178 tracer was then competed-off by increasing amounts of inhibitors in titration experiments in 96-well plates. Competition was detected by loss of TR-FRET signal. The signal was produced by a FRET pair between an Eu-labeled anti his-tag antibody, which recognizes his-tagged BRAF used in the assay, and the T178 tracer. The europium donor was excited using a 340 nm excitation filter and energy transfer to the T178 tracer was measured using a filter centered at 665 nm with a time delay of 200 μs. The emission ratio was calculated as the 665 nm signal divided by the 615 nm signal. The apparent % inhibition was calculated by least squares fits of the emission ratio. Data were normalized to 0 and 100% saturation and were transformed to true IC₅₀ values using the Cheng-Prusoff equation and the determined Kd value for BRAF-tracer interaction under the same conditions of 25 nM. The maximum DMSO concentration in the assay was 2%.

### In Vitro Residence Times of Inhibitors

Residence times of inhibitors to recombinant full-length BRAF was determined using an adaptation of the LanthaScreen Eu Kinase Binding Assay (Invitrogen). Initially, binding of inhibitors to his-tagged BRAF labelled with Eu-anti-his antibody at 80-90% saturation was established, by incubating inhibitors and BRAF in PBS buffer (max DMSO 2%) for 30 mM The reactions were then rapidly diluted 25× times in a saturated concentration of the fluorescent ATP-site tracer T178 (Invitrogen). Dissociation of inhibitors from BRAF by T178 tracer was monitored in real-time by detecting the TR-FRET 665 nm to 665 nm emission ratio every 20 sec. The TR-FRET signal was normalized between 100% (no inhibitors present) and 0% (saturated inhibitor binding). Time traces were fit to a single exponential to obtain the half-life (t_{1/2}) of dissociation of inhibitors. Residence times for each inhibitor were calculated as (t_{1/2})/ln2.

### Cellular Engagement of Inhibitors by Cellular Thermal Shift Assay (CETSA) Analysis

For CETSA analysis, cultured A375 melanoma cells were washed with Dulbecco's phosphate buffered saline (DPBS) and split into 500 μL aliquots (each containing 3,75 million cells) in the same buffer, containing DMSO control (20 μM) or 1, 5 and 20 μM of DAB or DABK. The samples were incubated for 1 hr at room temperature, rotating. After compound incubation, samples (50 μL each) were transferred in PCR tubes and incubated for 3 min in a temperature gradient produced with a C1000 thermal cycler (Bio-Rad). Cells were immediately lysed by repeating freeze-thaw cycles (3× times) in liquid nitrogen. Lysates were spun in a microcentrifuge at 15.000× g for 15 min at 4° C. Equal volumes of supernatants were run on 15-well 4-12% NuPAGE SDS-PAGE gels (Invitrogen), and analyzed by western blot. Results were quantitated by densitometric analysis and were normalized to GAPDH loading control, which is temperature insensitive under these conditions. Tm values were derived by least square fits of normalized CETSA curves.

### Pharmacokinetic (PK) and Pharmacodynamic (PD) Analysis

The pharmacokinetic profile of DAB and DABK was assessed in CD-1 female mice after a single dose at 5 mg/kg by oral gavage. Blood samples were collected at various time points (0.5, 1, 2, 4, 6, 12 and 24 hr after oral gavage) and inhibitor concentrations in plasma determined by an internal standard HPLC-chromatography tandem mass spectrometry method using calibration standards prepared in blank mouse plasma. Reported plasma concentrations are average values from 3 mice per time point. For PD study, tumor xenografts were established by subcutaneous implantation of A375 melanoma cells plus Matrigel (BD Biosciences) into the right flank of female SCID mice. Mice were randomized to treatment and control groups when the average tumor volume reached 100-150 mm³ and were treated with a single oral dose (PO) of either vehicle or inhibitors at 5 mg/kg. Tumors were harvested at 2, 12 and 24 hr post PO (3 mice per group). Harvested tumors were homogenized in in lysis buffer containing 50 mM Tris-HCl pH7.5, 1% NP40, 150 mM NaCl, 1 mM EDTA, 10% glycerol and phosphatase/protease inhibitors. pERK levels in clarified lysates were determined by Western Blotting and were expressed as % inhibition by normalization to average levels from vehicle tumors (0% inhibition). Vehicle formulation for DAB and DABK treatment in both PK and PD was 30% PEG-400, 0.5% Tween-20, 5% Glycerol in PBS.

### EXAMPLES

### Abbreviations

The following abbreviations may be used in the examples or elsewhere in the specification.

| | | | |
|---|---|---|---|
| AcOH | Acetic Acid | MTBE | Methyl tert-butyl ether |
| Bn | Benzyl | NBS | N-Bromosuccinimide |
| DCM | Dichlormethane | NCS | N-Chlorosuccinimide |
| DIEA | N,N-Diisopropylethylamine | NMP | N-Methyl-2-pyrrolidone |
| DMA | Dimethylacetamide | Pd₂(dba)₃ | Tris(dibenzylideneacetone) |
| | | | dipalladium |
| DMF | Dimethylformamide | Py | Pyridine |
| Et₃N | Triethylamine | TBS | Tert-Butyldimethhyl silyl |
| EtOAc | Ethylacetate | TEA | Triethanolamine |
| HATU | Hexafluorophosphate | TFA | Trifluoro acetic acid |
| | Azabenzotriazole | | |
| | Tetramethyl Uronium | | |
| LiHMDS | Lithium | THF | Tetrahydrofuran |
| | bis(trimethylsilyl)amide | | |
| | | Xantphos | 9,9-Dimethyl-9H-xanthene- |
| | | | 4,5-diyl)bis |
| | | | (diphenylphosphane) |

### General Procedures

### Chemical Synthesis

All chemical reagents and solvents were obtained from commercial sources and used without further purification. Microwave reactions were performed using an Anton Paar Monowave 300 reactor. Chromatography was performed on a Teledyne ISCO CombiFlash R_{f} 200i using disposable silica cartridges. Analytical thin layer chromatography (TLC) was performed on Merck silica gel plates and compounds were visualized using UV or CAM. NMR spectra were recorded on Bruker 300 and 600 spectrometers. The Bruker 600 NMR instrument was purchased using funds from NIH award 1S10OD016305. ¹H chemical shifts (δ) are reported relative to tetramethyl silane (TMS, 0.00 ppm) as internal standard or relative to residual solvent signals. Mass spectra were recorded by the Proteomics Facility at the Albert Einstein College of Medicine.

### Example 1. Synthesis of DAB-K

DABK is prepared according to the following synthetic scheme. SI4 was synthesized according to the procedure of Huang, S. et al., (CA2771775C, issued Jan. 20, 2015).

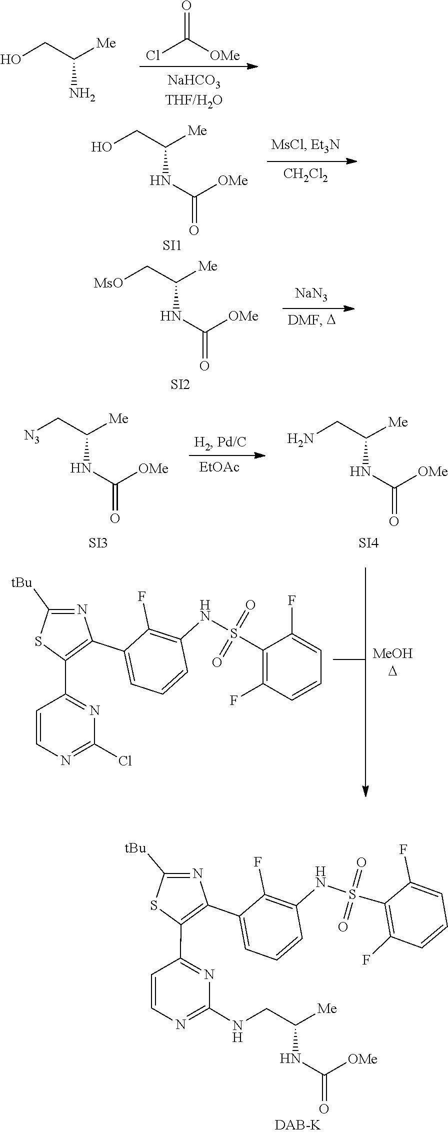

Step 1. Preparation of Methyl (S)-(1-hydroxypropan-2-yl)carbamate (SI1—Synthetic Intermediate 1)

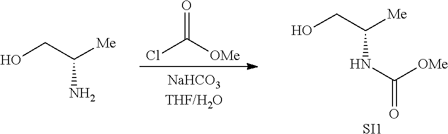 [SMILES: C[C@H](N)CO ; COC(=O)Cl ; COC(=O)N[C@@H](C)CO ; "SI1"]

Water (150 mL), THF (150 mL) and (S)-2-aminopropan-1-ol (5.00 mL, 64 mmol, 1.0 equiv.) were added to a flask and sodium bicarbonate (16 g, 190 mmol, 3 equiv.) was then added. The flask was cool in a water/ice bath and methyl chloroformate (5.5 mL, 71 mmol, 1.1 equiv.) was added slowly. The reaction mixture was allowed to slowly warm to room temperature, and after 3.5 hours, the mixture was diluted with EtOAc (100 mL) and transferred to a separatory funnel. The phases were separated and the aqueous phase was extracted with EtOAc (2×40 mL). The combined organic phases were dried (Na₂SO₄), filtered, and concentrated. The crude product (4.30 g, 32 mmol, 50%) was used without further purification.

Step 2. Synthesis of (S)-2-((Methoxycarbonyl)amino)propyl methanesulfonate (SI2)

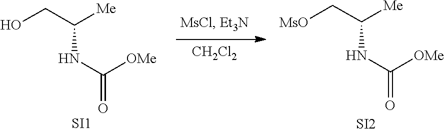

A flask with crude alcohol SI1 (5.80 g, 43.6 mmol, 1.0 equiv.) was purged with argon. CH₂Cl₂ (100 mL) was added and the flask was closed with a septum/Ar balloon. The flask was cooled in a water/ice bath and triethylamine (15 mL, 109 mmol, 2.5 equiv.) was added followed by drop-wise addition of MsCl (5.09 mL, 65.3 mmol, 1.5 equiv.). Stirring continued for 2.5 hours before the reaction was quenched by the addition of water. The phases were separated, and the aqueous phase was extracted once with EtOAc. The combined organic phases were then washed with 1 M NaOH and brine, dried (Na₂SO₄), filtered, and concentrated. The crude mesylate SI2 was used directly.

Step 3. Synthesis of Methyl (S)-(1-azidopropan-2-yl)carbamate (SI3)

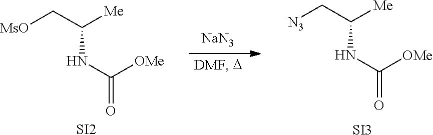

To a flask with crude mesylate SI2 (9.1 g, 43 mmol, 1.0 equiv.) was added DMF (60 mL) and sodium azide (4.5 g, 69 mmol, 1.6 equiv.). The flask was then placed in a pre-heated oil-bath (80° C.) with stirring for 10 minutes. After cooling to room temperature water and brine were added, and the product was extracted with EtOAc (3×). The combined organic phases were washed once with brine, dried over Na₂SO₄, filtered, and concentrated. The residue was loaded on a silica cartridge with hexanes/CH₂Cl₂ and then purified by column chromatography (24 g silica, 0-50% EtOAc in hexanes) resulting in azide SI3 (4.12 g, 26.0 mmol, 61% over 2 steps).TLC: R_{f}=0.48 (Hexanes/EtOAc 1:1; CAM). ¹H NMR (300 MHz, CDCl₃): δ 4.71 (bs, 1H), 4.00-3.85 (m, 1H), 3.70 (s, 3H), 3.46 (dd, J=12.1, 4.3 Hz), 3.38 (dd, J=12.1, 4.6 Hz, 1H), 1.23 (d, J=6.8 Hz, 3H).

Step 4. Synthesis of Methyl (S)-(1-aminopropan-2-yl)carbamate (SI4)

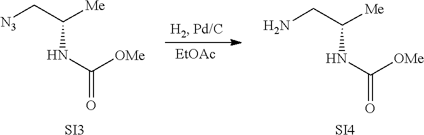 [SMILES: "EtOAc" ; COC(=O)N[C@@H](C)CN=[N+]=[N-] ; "SI3" ; "H2, Pd/C" ; COC(=O)N[C@@H](C)CN ; "SI4"]

Azide SI3 (1.00 g, 6.32 mmol, 1.00 equiv.) was dissolved in EtOAc (50 mL) and then the flask was purged with argon. Palladium on carbon (Degussa, 50% water, 5% Pd, 100 mg, 47 0.7 mol %) was added and the flask was sealed. The argon atmosphere was replaced with hydrogen by 3 cycles of house vacuum/H₂ balloon. After 16 hours, TLC analysis (Hexanes:EtOAc 1:1 CAM) showed incomplete conversion. Addition Pd/C (100 mg, 47 μmol 0.7 mol %) was added the hydrogen was introduced as before. After an additional 16 hours, TLC analysis showed full conversion. The mixture was filtered through celite, which was then rinsed with EtOAc. Removal of the volatiles gave amine SI4 (830 mg, 6.32 mmol, quant.).

TLC: R_{f}=0.54 (CH₂Cl₂/MeOH/NH₄OH 75:23:3; KMnO₄). ¹H NMR (300 MHz, CDCl₃): δ 4.84 (bs, 1H), 3.75-3.65 (m, 4H), 2.78 (dd, J=12.8, 5.0 Hz, 1H), 2.66 (dd, J=12.9, 6.3 Hz, 1H), 1.15 (d, J=6.7 Hz, 3H). ¹³C NMR (DMSO-d6, 151 MHz): δ 156.7, 51.5, 49.5, 47.2, 18.7.

### Step 5. Synthesis of DAB-K

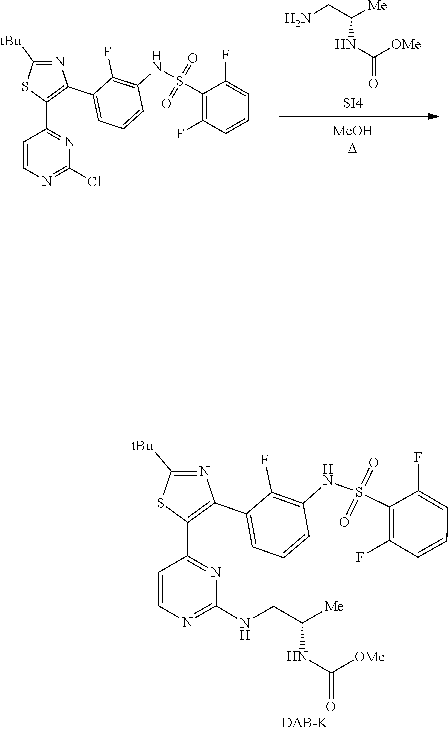 [SMILES: CC(C)(C)c1nc(-c2cccc(NS(=O)(=O)c3c(F)cccc3F)c2F)c(-c2ccnc(Cl)n2)s1 ; COC(=O)N[C@@H](C)CN ; "SI4" ; COC(=O)N[C@@H](C)CNc1nccc(-c2sc(C(C)(C)C)nc2-c2cccc(NS(=O)(=O)c3c(F)cccc3F)c2F)n1]

N-(3-(2-(tert-Butyl)-5-(2-chloropyrimidin-4-yethiazol-4-yl)-2-fluorophenyl)-2,6-difluorobenzene-sulfonamide (150 mg, 0.28 mmol, 1 equiv.) and amine SI4 (185 mg, 1.40 mmol, 5 equiv.) were add to a microwave vial followed by methanol (20 mL). The vial was then capped and heated to 110° C. for 4 hours in the microwave reactor. The volatiles were then removed under reduced pressure and residual methanol was removed by co-evaporation with toluene. The residue was loaded on silica with CH₂Cl₂ and purified by column chromatography (4 g silica, 0-35% EtOAc in CH₂Cl₂) giving DAB-K (85 mg, 0.13 mmol, 48%).

TLC: R_{f}=0.24 (CH₂Cl₂/EtOAc 2:1; UV). ¹H NMR (600 MHz, CDCl₃): δ 7.93 (bs, 1H), 7.70 (t, J=7.8 Hz, 1H), 7.50-7.44 (m, 1H), 7.36 (t, J=6.4 Hz, 1H), 7.21 (t, J=8.0 Hz, 1H), 6.96 (t, J=8.8 Hz, 2H), 6.13 (bs, 1H), 5.18 (bs, 1H), 3.89 (bs, 1H), 3.62 (3.61, 3H), 3.40 (bs, 1H), 3.30 (bs, 1H), 1.47 (s, 9H), 1.17 (d, J=6.4 Hz, 3H). ¹³C NMR (151 MHz, CDCl₃): δ 182.7, 162.2, 159.8 (dd, J=259.3, 3.2 Hz), 160.6 (d, J=3.2 Hz), 158.9 (d, J=3.3 Hz), 158.6 (bs), 158.1, 156.8, 152.4 (d, J=249.6 Hz), 145.8, 135.1 (t, J=11. 2 Hz), 133.8, 128.4, 125.0 (d, J=4.4 Hz) , 124.6-124.3 (m), 123.9 (bs), 117.1 (t, J=15.4 Hz), 52.0, 47.7, 46.5, 38.0, 18.6, 14.2. ¹⁹F NMR (565 MHz, CDCl₃): δ −106.8, −128.9. HRMS calculated for C28H30F3N6O4S2 635.1717 found 635.1712.

### Example 2. Synthesis of Compound K5

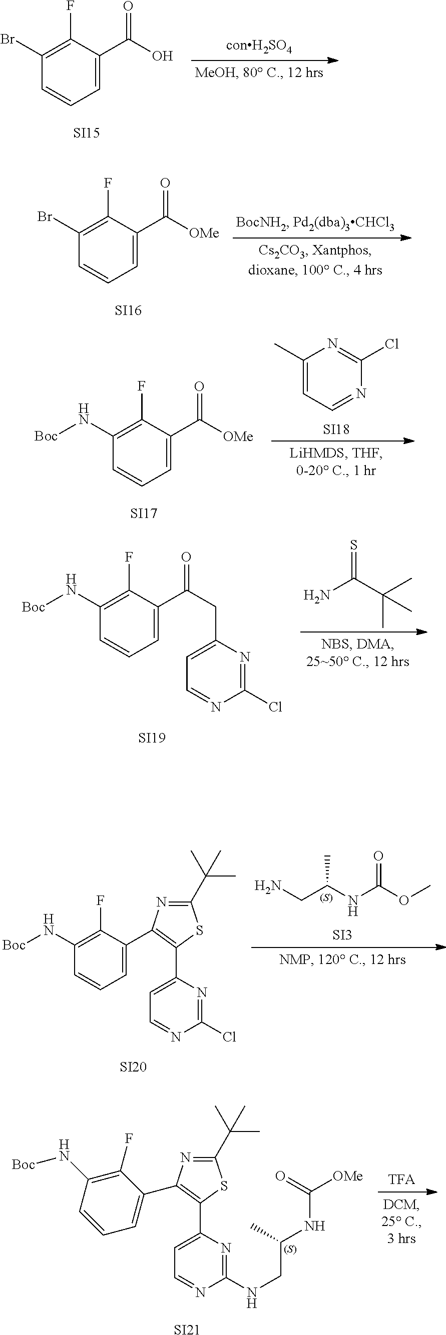

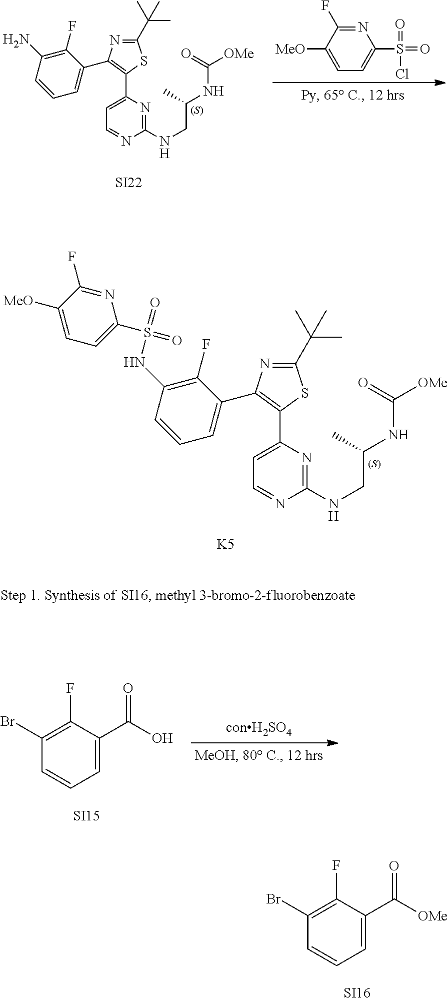

Concentrated.H₂SO₄ (11.2 g, 114 mmol, 1.00 eq) was added to a mixture of compound SI15 (25.0 g, 114 mmol, 1.00 eq) in MeOH (200 mL). Then the mixture was stirred at 80° C. for 12 hrs under N₂ atmosphere. LCMS (EC1723-1-P1A2) showed one peak (RT=0.561 min) with desired MS=232.8 was detected. The reaction solution was concentrated to remove solvent. Water (200 ml) was added to the reaction mixture, and then the reaction mixture was extracted with ethyl acetate (100 mL*2). The combined organic layers were washed with brine (200 mL), dried over Na₂SO₄, filtered, and concentrated to give a compound SI16 (24.6 g, crude) as a yellow oil.

LCMS: EC1723-1-P1A2, RT=0.211 min, M/Z (ESI): 232.8 (M+H)⁺.

Step 2. Preparation of Compound SI17, methyl 3-((tert-butoxycarbonyl)amino)-2-fluorobenzoate

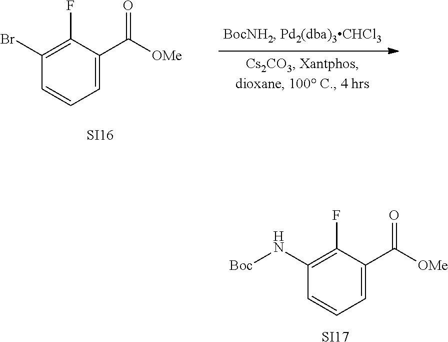

To a mixture of compound SI16 (10.0 g, 129 mmol, 1.00 eq), BocNH₂ (7.54 g, 64.4 mmol, 1.50 eq), Cs₂CO₃ (28.0 g, 85.8 mmol, 2.00 eq), Xantphos (2.48 g, 4.29 mmol, 0.10 eq) and Pd₂(dba)₃ (3.93 g, 4.29 mmol, 0.10 eq) in dioxane (100 mL) was degassed and purged with N₂ for 3 times, then the mixture was stirred at 100° C. for 4 hrs. under N₂ atmosphere. TLC (Petroleum ether:Ethyl acetate=5:1, R1 R_{f}=0.58, P1 R_{f}=0.5) indicated reactant was consumed completely, and one major new spot with larger polarity was detected. The reaction mixture was quenched by addition water 100 mL, and extracted with ethyl acetate (200 mL*3). The combined organic layers were washed with brine (100 mL), dried over Na₂SO₄, filtered, and concentrated under reduced pressure to give a residue. The residue was purified by silica gel chromatography eluted with Petroleum ether:Ethyl acetate=20:1 to 10:1. Compound SI17 (10.6 g, 39.4 mmol, 91.7% yield) was obtained as a yellow solid.

¹HNMR: EC1797-4-P1A, 400 MHz, CDCl₃δ 1.51-1.57 (m, 9 H) 3.90-3.96 (m, 3 H) 6.76-6.83 (m, 1 H) 7.13-7.19 (m, 1 H) 7.51-7.58 (m, 1 H) 8.27-8.37 (m, 1 H)

Step 3. Preparation of Compound SI19, tert-butyl (3-(2-(2-chloropyrimidin-4-yl)acetyl)-2-fluorophenyl)carbamate

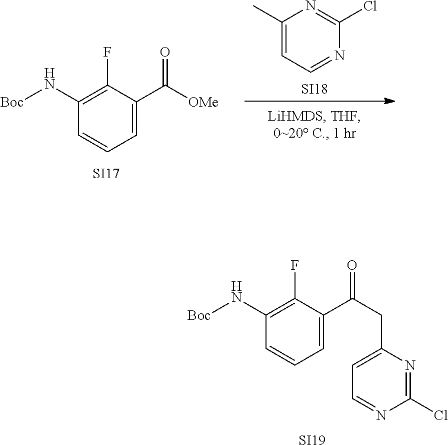

A mixture of compound SI17 (10.0 g, 37.1 mmol, 1.00 eq) in THF (100 mL) was degassed and purged with N₂ for 3 times and cooled to 0° C. Then LiHMDS (1.0 m, 74.28 mL, 2.00 eq) and compound SI18 (6.21 g, 48.28 mmol, 1.3 eq) were added at 0° C. The mixture was stirred at 20° C. for 1 hr. under N₂ atmosphere. TLC (Petroleum ether:Ethyl acetate=5:1, R1 R_{f}=0.8, P1 R_{f}=0.3) indicated reactant was consumed completely and one new spot formed. The reaction mixture was poured to ice water (100 mL) and 1 M HCl was added to pH=4. The mixture was extracted with ethyl acetate (100 mL*2), organic layer separated, and concentrated under reduced pressure. The residue was purified by column chromatography (SiO₂, Petroleum ether:Ethyl acetate=20:1 to 0:1). Compound SI19 (9.00 g, 24.6 mmol, 72.2% yield, 66.3% purity) was obtained as a yellow solid.

Step 4. General Procedure for Preparation of Compound SI20, tert-butyl (3-(2-(tert-butyl)-5-(2-chloropyrimidin-4-yl)thiazol-4-yl)-2-fluorophenyl)carbamate

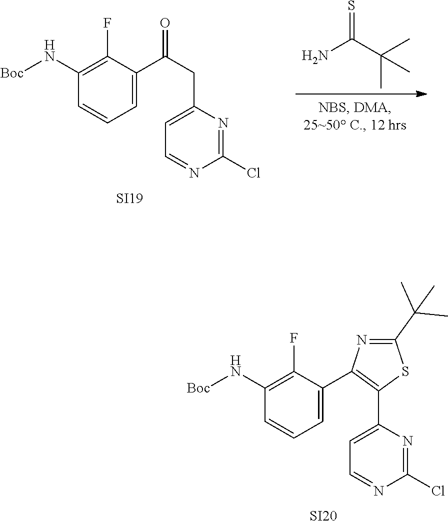

A mixture of compound SI19 (8.50 g, 23.2 mmol, 1.00 eq), NBS (4.55 g, 25.5 mmol, 1.00 eq) in DMA (100 mL) was stirred at 25° C. for 15 min, then the 2,2-dimethylpropanethioamide (2.98 g, 25.4 mmol, 1.09 eq) was added. The mixture was stirred at 50° C. for 12 hrs. under N₂ atmosphere. TLC (Petroleum ether:Ethyl acetate=3:1, R1 R_{f}=0.4, P1 R_{f}=0.7) indicated reactant was consumed completely and one new spot formed. The reaction mixture was quenched by addition water 150 mL at 20° C., extracted with ethyl acetate (150 mL*3), the combined organic layers were washed with brine (100 mL), dried over Na₂SO₄, filtered, and concentrated under reduced pressure to give a residue. The residue was purified by column chromatography (SiO₂, Petroleum ether:Ethyl acetate=40:1). The Compound SI20 was obtained (6.00 g, 13.0 mmol, 55.7% yield) as a yellow solid.

¹H NMR: EC1797-11-P1A, 400 MHz, CDCl₃.δ 1.48-1.50 (m, 9 H) 1.51-1.53 (m, 9 H) 2.53-2.56 (m, 4 H) 6.88-6.92 (m, 1 H) 7.11-7.14 (m, 1 H) 7.21-7.26 (m, 1 H) 8.34-8.37 (m, 1 H) 8.45-8.49 (m, 1 H)

Step 5. Preparation of Compound SI21, Methyl (S)-(1-((4-(4-(3-((tert-butoxycarbonyl)amino)-2-fluorophenyl)-2-(tert-butyl)thiazol-5-yl)pyrimidin-2-yl)amino)propan-2-yl)carbamate

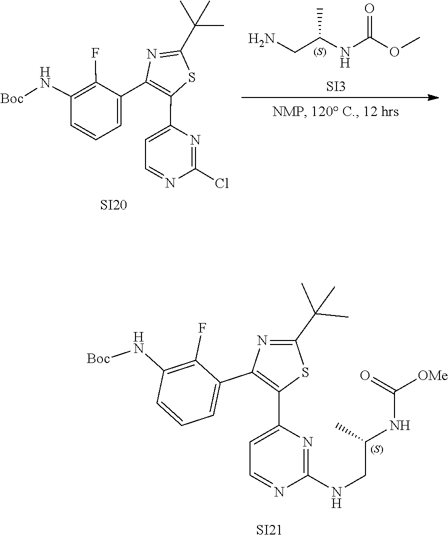

A mixture of compound SI20 (2.00 g, 4.32 mmol, 1.00 eq), SI3 (801 mg, 4.75 mmol, 1.10 eq, HCl) and DIEA (1.67 g, 12.96 mmol, 3.00 eq) in NMP (25 mL) was prepared. The mixture was stirred at 120° C. for 12 hrs. under N₂ atmosphere. TLC (Petroleum ether:Ethyl acetate=1:1, R1 R_{f}=0.8, P1 R_{f}=0.3) indicated reactant was consumed completely and one new spot formed. LCMS (EC1797-13-P1A) showed one peak (RT=0.712 min) with desired MS=559.2 was detected. The reaction mixture was quenched by addition water (50 mL) at 20° C., extracted with ethyl acetate (50 mL*3), the combined organic layers were washed with brine (100 mL), dried over Na₂SO₄, filtered, and concentrated under reduced pressure to give a residue. The residue was purified by column chromatography (SiO₂, Petroleum ether:Ethyl acetate=10/1 to 1/1). Compound SI21 (1.90 g, 3.06 mmol, 70.8% yield, 90% purity) was obtained as a yellow oil.

LCMS: EC1797-13-P1A, RT=0.712 min, M/Z (ESI): 559.2 (M+H)⁺. ¹H NMR: EC1797-13-P1A,400 MHz, CDCl₃ δ 1.49-1.51 (m, 9 H) 1.52-1.55 (m, 9 H) 2.33-2.37 (m, 4 H) 3.62-3.66 (m, 3 H) 6.27-6.32 (m, 1 H) 7.18-7.23 (m, 1 H) 8.06-8.09 (m, 1 H)

### Step 6. Preparation of Compound SI22

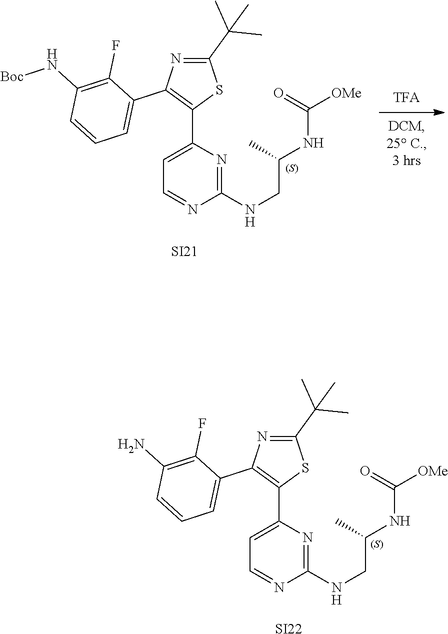

A mixture of compound SI21 (1.7 g, 3.04 mmol, 1.00 eq) and TFA (1.73 g, 15.21 mmol, 5.00 eq) in DCM (5 mL) was prepared. The mixture was stirred at 25° C. for 3 hrs. under N₂ atmosphere. TLC (Petroleum ether:Ethyl acetate=1: 1, R1 R_{f}=0.7, P1 R_{f}=0.4) indicated reactant was consumed completely and one new spot formed. LCMS (EC1797-18-P1A) showed one peak (RT=0.584 min) with Desired MS=459.1 was detected. The reaction mixture was quenched by addition Na₂CO₃ (aqueous) to pH=8, extracted with DCM (10 mL*3). The combined organic layers were washed with brine (10 mL), dried over Na₂SO₄, filtered, and concentrated under reduced pressure to give a residue. The residue was purified by column chromatography (SiO₂, Petroleum ether:Ethyl acetate=5:1 to 1/1). Compound 22 (1.30 g, 2.84 mmol, 93.2% yield) as a yellow oil.

LCMS: EC1797-18-P1A, RT=0.584 min, M/Z (ESI): 559.1 (M+H)⁺. ¹H NMR: EC1797-18-P1A, 400 MHz, CDCl₃δ 1.17-1.24 (m, 4 H) 1.47-1.53 (m, 9 H) 1.97-2.07 (m, 2 H) 2.35-2.41 (m, 2 H) 2.83-2.88 (m, 2 H) 3.36-3.41 (m, 2 H) 3.61-3.67 (m, 4 H) 6.32-6.39 (m, 1 H) 6.81-6.91 (m, 2 H) 6.98-7.06 (m, 1 H) 8.03-8.08 (m, 1 H).

Step 8. Preparation of Compound K5, methyl (S)-(1-((4-(2-(tert-butyl)-4-(2-fluoro-3-((6-fluoro-5-methoxypyridine)-2-sulfonamido)phenyl)thiazol-5-yl)pyrimidin-2-yl)amino)propan-2-yl)carbamate

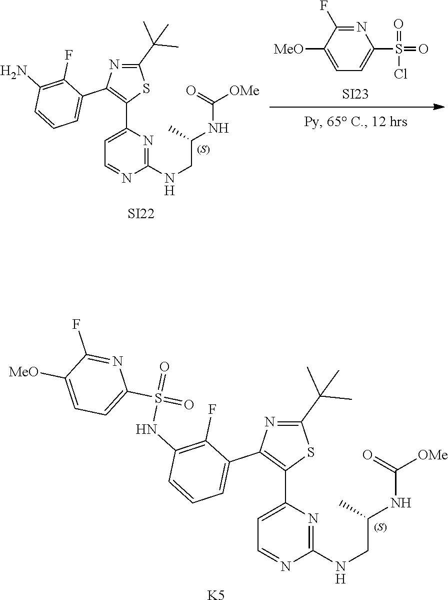

A mixture of compound SI22 (0.10 g, 218 μmol, 1.00 eq) and SI23 (98.4 mg, 436 μmol, 2.00 eq, synthesis details given in Ex. 5) in pyridine (2 mL) was prepared. The mixture was stirred at 65° C. for 12 hrs. under N₂ atmosphere. TLC (Petroleum ether:Ethyl acetate=1:1, R1 R_{f}=0.8, P1 R_{f}=0.3) indicated reactant was consumed completely and one new spot formed. LCMS (EC1797-25-P1A) showed one peak (RT=0.545 min) with desired MS=648.0 was detected. The reaction mixture was filtered to give a clear solution. The residue was purified by prep-HPLC (FA condition). The Compound K5 (0.02 g, 30.3 μmol, 13.9% yield) was obtained as a yellow oil.

¹H NMR: EC1797-25-P1A (400 MHz, CDCl₃) δ 1.20 (d, J=6.63 Hz, 3 H) 1.47-1.49 (m, 9 H) 1.51 (s, 1 H) 3.36-3.50 (m, 2 H) 3.61-3.67 (m, 3 H) 3.96 (s, 4 H) 6.23-6.30 (m, 1 H) 7.16-7.22 (m, 1 H) 7.28 (s, 1 H) 7.65-7.72 (m, 1 H) 7.81-7.86 (m, 1 H) 7.97-8.02 (m, 1 H)

LCMS: EC1797-25-P1Z2, RT=0.546 min, M/Z (ESI): 648.1 (M+H)⁺. HPLC: EC1797-25-P1Z, RT=3.264 min, 98.1% purity. SFC: EC1797-25-P1C1, % ee=100

### Example 3. Preparation of Compound K7, Methyl (S)-(1-((4-(2-(tert-butyl)-4-((3(N-ethyl-N-methylsulfamoyl)amino)-2-fluorophenyl)thiazol-5-yl)pyrimidin-2-yl)amino)propan-2-yl)carbamate

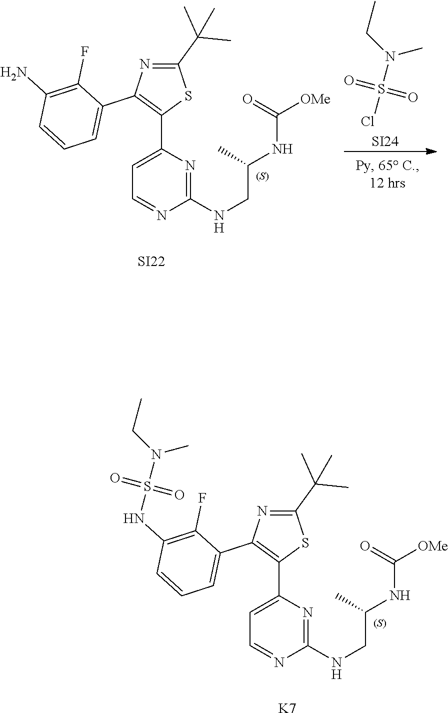

A mixture of compound SI22 (0.2 g, 436 μmol, 1 eq) in pyridine (3 mL) was degassed and purged with N₂ for 3 times, and then compound SI24 (137.49 mg, 872.32 μmol, 2 eq) was added to the mixture was stirred at 65° C. for 12 hr. under N₂ atmosphere. LCMS (EC1797-24-P1A2) showed reactant was consumed completely and one main peak with desired mass was detected. The reaction mixture was filtered to give a clear solution.

The residue was purified by prep-HPLC (FA condition) to give compound K7 (0.06 g, 97.8 μmol, 22.4% yield, 94.52% purity) as a white solid. The solid was further purified by prep-HPLC (column Waters xbridge 150*25 mm 10 um; mobile phase: [water (10 mM NH₄HCO₃)-ACN]; B %: 40%-70%, 11 min) to give compound K7 (0.018 g, 30.7 μmol, 29.7% yield, 99% purity) as a white solid.

¹H NMR: EC1797-24-P1A, 400 MHz, CDCl₃δ 7.97 (br d, J=4.1 Hz, 1H), 7.52 (dt, J=1.5, 7.8 Hz, 1H), 7.29-7.21 (m, 1H), 7.18-7.12 (m, 1H), 6.71 (br s, 1H), 6.25 (br d, J=4.3 Hz, 1H), 5.22-4.88 (m, 1H), 3.90-3.74 (m, 1H), 3.56 (s, 3H), 3.46-3.33 (m, 1H), 3.23-3.12 (m, 2H), 2.76 (s, 3H), 1.43 (s, 9H), 1.15-1.04 (m, 6H). LCMS: EC1797-40-P1B2, RT=0.543 min, M/Z (ESI): 580.1 (M+H)⁺. HPLC:EC1797-40-P1B1, RT=3.215 min, 97.2% purity

SFC: EC1797-40-P1C2_A1, % ee=100

### Example 4. Preparation of Compound K9, Methyl (S)-(1-((4-(2-(tert-butyl)-4-(2-fluoro-3-(pyrrolidine-1-sulfonamido)phenyl)thiazol-5-yl)pyrimidin-2-yl)amino)propan-2-yl)carbamate

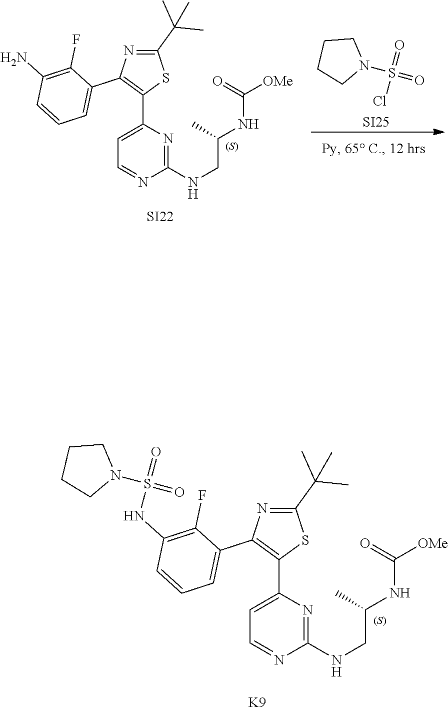

A mixture of compound SI22 (0.2 g, 436.16 μmol, 1 eq) in pyridine (3 mL) was degassed and purged with N₂ for 3 times, and then compound SI25 (147.97 mg, 872.31 μmol, 2 eq) was added. The mixture was stirred at 65° C. for 12 hrs. under N₂ atmosphere. LCMS (EC1797-23-P1A2) showed reactant was consumed completely and one main peak with desired mass was detected. The reaction mixture was concentrated to give a residue. The residue was purified by prep-HPLC (FA condition) to give compound K9 (0.11 g, 168 μmol, 38.5% yield, 99.3% purity) as a white solid.

¹H NMR: EC1797-23-P1A, 400 MHz, CDCl₃ δ 8.07 (d, J=5.3 Hz, 1H), 7.66 (dt, J=1.6, 7.8 Hz, 1H), 7.37-7.30 (m, 1H), 7.26-7.20 (m, 1H), 6.86 (br s, 1H), 6.32 (br d, J=5.1 Hz, 1H), 5.28-4.97 (m, 1H), 4.04-3.82 (m, 1H), 3.66 (s, 3H), 3.55-3.42 (m, 1H), 3.41-3.27 (m, 5H), 1.92-1.83 (m, 4H), 1.52 (s, 9H), 1.21 (d, J=6.6 Hz, 3H). LCMS: EC1797-23-P1Z1, RT=0.548 min, M/Z (ESI): 592.1 (M+H)⁺. HPLC: EC1797-23-P1Z, RT=3.268 min, 99.3% purity SFC: EC1797-P1C1_A 2_A1, ee=100%

### Example 5. Preparation of SI23, 6-fluoro-5-methoxypyridine-2-sulfonyl chloride

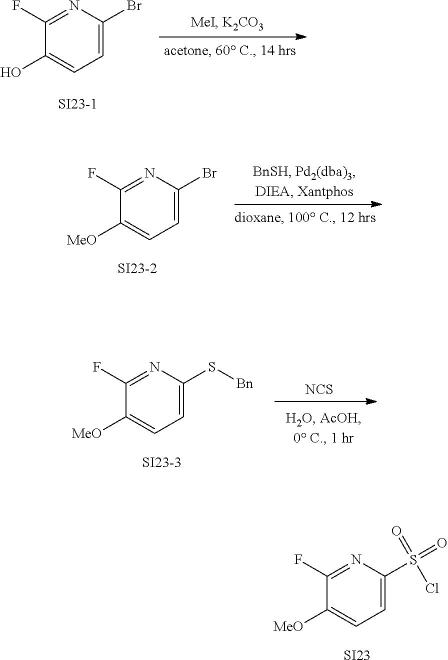

Step 1. Preparation of Compound 5b-2, 6-Bromo-2-fluoro-3-methoxypyridine

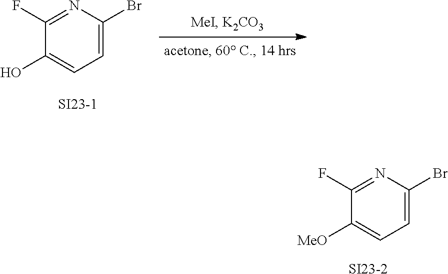

To a mixture of compound SI23-1 (2.00 g, 10.4 mmol, 1.00 eq) in acetone (30 mL) was added K₂CO₃ (2.88 g, 20.8 mmol, 2.00 eq) and CH₃I (2.96 g, 20.8 mmol, 2.00 eq). The mixture was stirred at 60° C. for 14 hrs. under N₂ atmosphere. LC-MS (EC1719-5-P1A1) showed one peak (RT=0.527 min) with desired MS=205.8. The reaction mixture filtered and concentrated under reduced pressure to give a residue. Compound SI23-2 (2.10 g, crude) was obtained as a yellow solid.

LCMS, EC1719-5-P1A1, RT=0.527 min, M/Z (ESI): 205.8 (M+H)+.

Step 2. Preparation of Compound SI23-3, 6-(Benzylthio)-2-fluoro-3-methoxypyridine

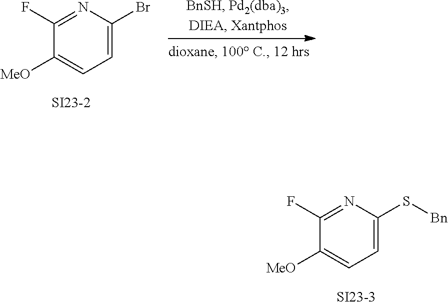

To mixture of compound SI23-2 (0.50 g, 2.42 mmol, 1.00 eq) in dioxane (100 mL) was added BnSH (449.97 mg, 3.62 mmol, 1.50 eq), DIEA (624 mg, 4.83 mmol, 2.00 eq), Xantphos (139 mg, 241, 0.10 eq) and Pd₂(dba)₃ (110 g, 121 μmol, 0.05 eq). The mixture was degassed and purged with N₂ 3 times, then the mixture was stirred at 100° C. for 12 hrs. under N₂ atmosphere. LCMS (EC1719-8-P1A1) showed one peak (RT=0.720 min) with desired MS=249.9. The reaction mixture was filtered and concentrated under reduced pressure to give a residue. The residue was purified by silica gel chromatography eluted with petroleum ether:Ethyl acetate (10:1 to 5:1). Compound SI23-3 (340 mg, crude) was obtained as a colorless oil. LCMS, EC1719-8-P1A1, RT=0.720 min, M/Z (ESI): 249.9 (M+H)⁺. ¹H NMR: EC1719-8-P1A1, 400 MHz, CDCl₃

Step 3. Preparation of Compound SI-23, 6-Fluoro-5-methoxypyridine-2-sulfonyl chloride

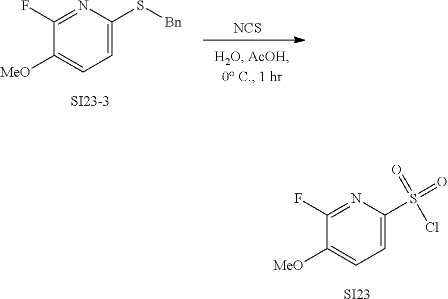

A mixture of sulfuryl chloride (3.90 g, 28.9 mmol, 4.00 eq) in H₂O (520 mg, 28.9 mmol, 4.00 eq) was added to compound SI23-3 (1.80 g, 7.22 mmol, 1.00 eq) in acetic acid (2.17 g, 36.1 mmol, 5.00 eq) and DCM (40 mL) at 0° C. The mixture was stirred at 0° C. for 1 hr. under N₂ atmosphere. LCMS (EC1719-18-P1A1) showed one peak (RT=0.533 min) with desired MS=225.8. The reaction mixture was quenched with saturated NaHCO₃ (50 mL), extracted with DCM (30 mL*3). The combined organic layers were washed with water (30 mL*2), dried over Na₂SO₄, filtered, and concentrated under reduced pressure to give a residue. Compound SI23 (1.50 g, crude) was obtained as an off-white solid.

LCMS: EC1719-18-P1A1, RT=0.533 min, M/Z (ESI): 225.8 (M+H)⁺.

### Example 6. Preparation of Compound K6

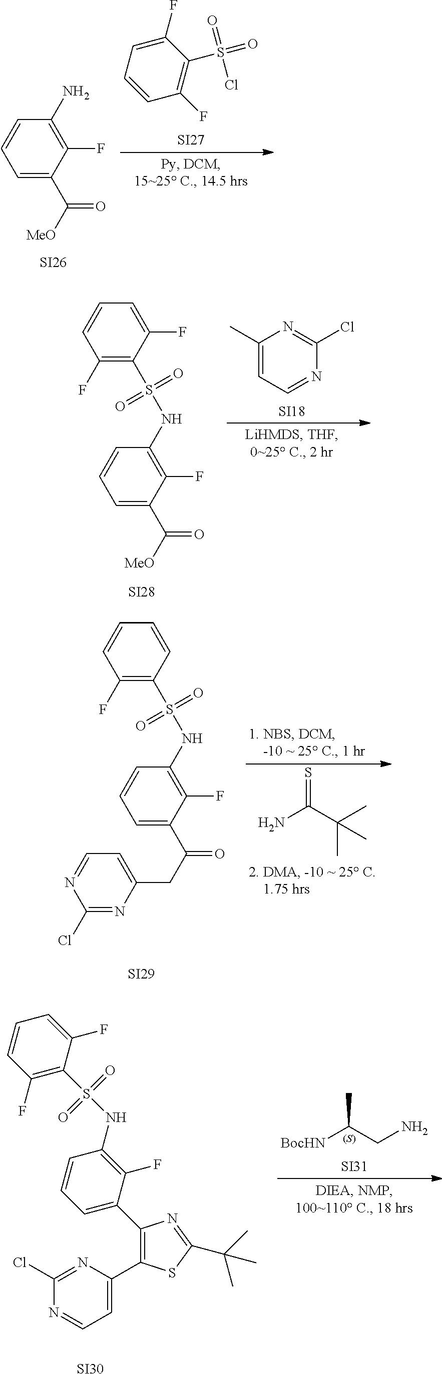

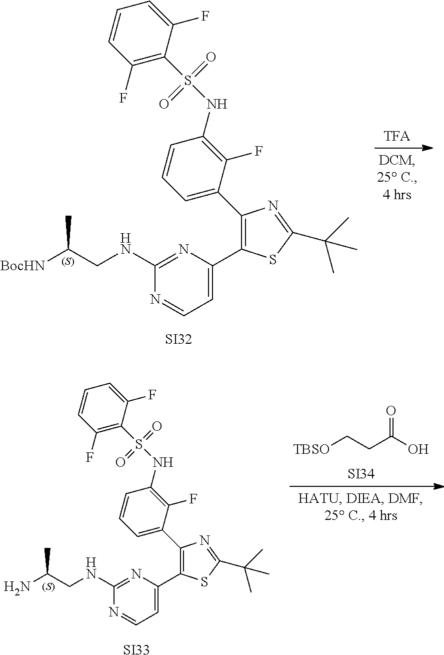

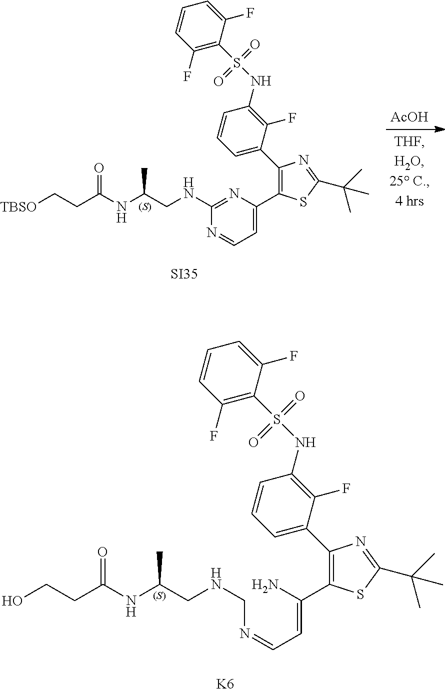

Step 1. Preparation of Compound SI28, Methyl 3-((2,6-difluorophenyl)sulfonamido)-2-fluorobenzoate

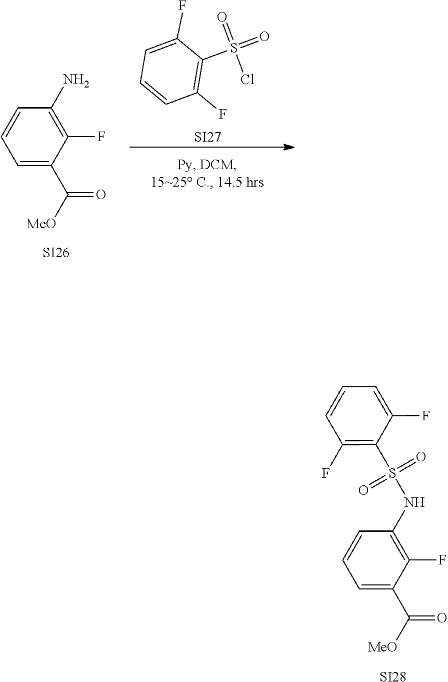

To a solution of compound SI26 (5.00 g, 29.5 mmol) in CH₂Cl₂ (25.0 mL) was added pyridine (3.51 g, 44.3 mmol, 3.58 mL) at 15˜25° C. and compound SI27 (6.28 g, 29.5 mmol, 4.00 mL). The mixture was stirred at 15˜25° C. for 14.5 h. TLC (Petroleum ether:Ethyl acetate=3:1 R1, R_{f}=0.30; P1, R_{f}=0.12) shows the staring material remained and a new spot was detected. LCMS (EC2025-1-IPC1, PDA =254 nm) shows 5.1% of the staring material (M+1=170, RT=0.348 min) remained, and 92.5% of desired compound (M+17=362.9, RT=0.495 min) was obtained. Water (25.0 mL) was added to the reaction solution, and stirred for 5 min. The solution was filtered, and the filter cake concentrated under reduced pressure at 40˜45° C. to give compound SI28 (4.85 g, 27.9 mmol, 98.2% purity) as a red solid

¹H NMR: EC2025-1-cake, 400 MHz, DMSO-d₆ δ 7.78-7.69 (m, 2H), 7.60-7.52 (m, 1H), 7.33-7.24 (m, 3H), 3.82 (s, 3H) LCMS: EC2025-1-IPC1, product: RT=0.495 min, m/z=362.9 (M+OH)⁺

Step 2. Preparation of Compound SI29, N-(3-(2-(2-chloropyrimidin-4-yl)acetyl)-2-fluorophenyl)-2,6-difluorobenzenesulfonami

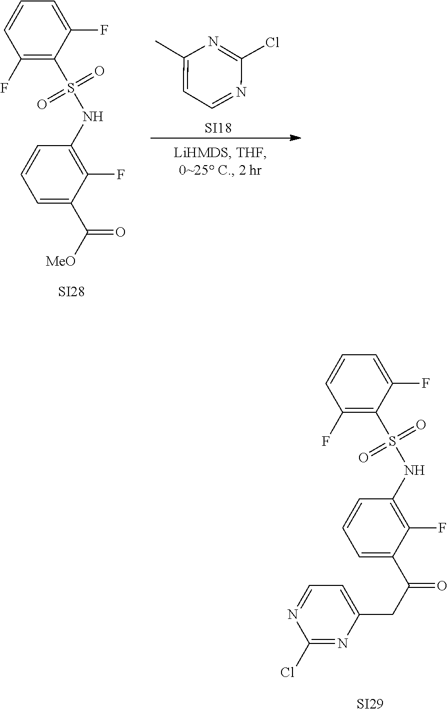

To a solution of compound SI28 (9.65 g, 27.9 mmol) in THF (75.0 mL) was added dropwise LiHMDS (1 M, 55.8 mL) at 0° C. under N₂. After addition, and then compound SI18 (5.39 g, 41.9 mmol) in 20.0 mL THF was added dropwise at 0° C. The resulting mixture was stirred at 2025° C. for 2 h. LC-MS (EC2025-7-P1X) showed of Reactant 1(M+17=363.1, RT=0.420 min) remained. Several new peaks were shown on LC-MS and desired compound (M+1=442.0, RT=0.430, 0.505 min) was detected. Charge 250 mL Ammonium chloride aqueous solution into the reaction solution and stir for 0.5 h. And extracted with Ethyl acetate (200 mL) (100 mL*2). The combined organic layers were washed with brine (100 mL) (50.0 mL*2), dried over Na₂SO₄, filtered, and concentrated under reduced pressure to give a residue. The residue was purified by flash silica gel chromatography (ISCO®; Sepa Flash® Silica Flash Column, Eluent of 0˜50% Ethyl acetate/Petroleum ether gradient). The solution was concentrated under reduced pressure to give compound SI29 (6.12 g, 13.8 mmol, crude) as a yellow solid.

¹H NMR: EC2025-7-P1, 400 MHz, DMSO-d₆ δ 8.74 (d, J=5.0 Hz, 1H), 8.64 (d, J=5.4 Hz, 1H), 7.75-7.72 (m, 1H), 7.68-7.62 (m, 1H), 7.61-7.58 (m, 1H), 7.56 (d, J=5.0 Hz, 1H), 7.50 (d, J=5.4 Hz, 1H), 7.46-7.40 (m, 1H), 6.14 (s, 1H), 4.49 (d, J=1.6 Hz, 2H) LCMS: EC2025-7-P1X, product: RT=0.430, 0.505 min, m/z=442.0 (M+H)⁺.

Step 3. Preparation of Compound SI30, N-(3-(2-(tert-butyl)-5-(2-chloropyrimidin-4-yl)thiazol-4-yl)-2-fluorophenyl)-2,6-difluorobenzenesulfonamide

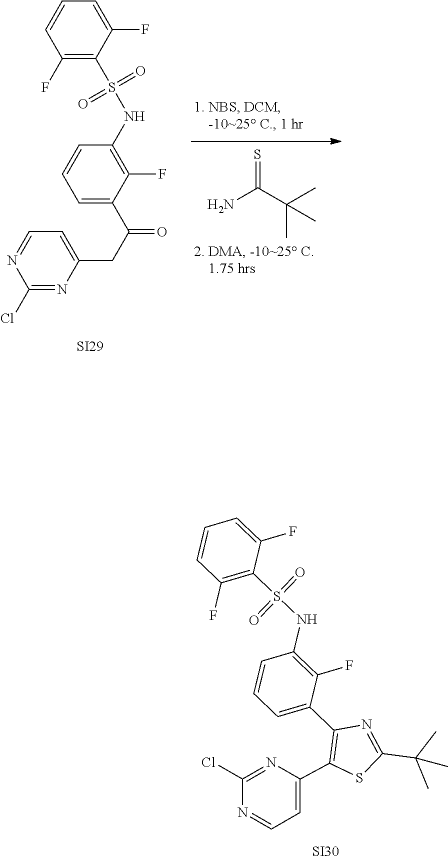

A solution of compound SI29 (4.10 g, 9.28 mmol) in DCM (40.0 mL) was cooled at −10° C. NBS (1.65 g, 9.28 mmol) was added and stirred for 1 h at 20˜25° C. Water (20.0 mL) was then added to the reaction vessel and the mixture was stirred and the layers separated. Water (20.0 mL) was added again to the dichloromethane layer and the mixture was stirred and the layers separated. Ethyl acetate (14.0 mL) was added to the reaction mixture and concentrated to 8.00 mL. DMA (36.0 mL) was then added to the reaction mixture and cooled to −10° C. The mixture was stirred at −10° C. and 2,2-dimethylpropanethioamide (543.8 mg, 4.64 mmol) added at 20˜25° C. and stirred 45 min. The vessel contents were heated to 75° C. and held at that temperature for 1 .75 hours. LC-MS (EC2025-10-P1C) showed of SI29 was consumed. Several new peaks were shown on LC-MS and the desired compound (M+1=539.1, RT=0.620 min) was detected. The reaction was cooled to 20˜25° C. The reaction mixture was quenched by addition of water (60 mL) at 25° C., and then diluted with ethyl acetate (70 mL) and extracted with ethyl acetate (80.0 mL, 40.0 mL*2). The combined organic layers were washed with brine (100 mL, 50 mL*2), dried over Na₂SO₄, filtered, and concentrated under reduced pressure to give SI30 as a residue. The residue (Petroleum ether: Ethyl acetate=2:1 R1: R_{f}=0.24; P1: R_{f}=0.29) was purified by flash silica gel chromatography (ISCO®; Sepa Flash® Silica Flash Column, Eluent of 0˜50% Ethyl acetate/Petroleum ether gradient). The solution was concentrated under reduced pressure to give compound SI30 (3.70 g, 6.32 mmol, 92% purity) as yellow solid.

¹H NMR: EC2025-10-p1, 400 MHz, CDCl₃ δ 8.30 (d, J=5.4 Hz, 1H), 8.00 (s, 1H), 7.73-7.65 (m, 1H), 7.55-7.45 (m, 1H), 7.39-7.32 (m, 1H), 7.27-7.21 (m, 1H), 6.98 (t, J=8.8 Hz, 2H), 6.77 (d, J=5.3 Hz, 1H), 1.48 (s, 9H). LCMS: EC2025-10-P1C, product: RT=0.620 min, m/z =539.1 (M+H)⁺.

Step 4. Preparation of Compound SI32, tert-butyl (S)-(1-((4-(2-(tert-butyl)-4-(3-((2,6-difluorophenyl)sulfonamido)-2-fluorophenyl)thiazol-5-yl)pyrimidin-2- yl)amino)propan-2-yl)carbamate

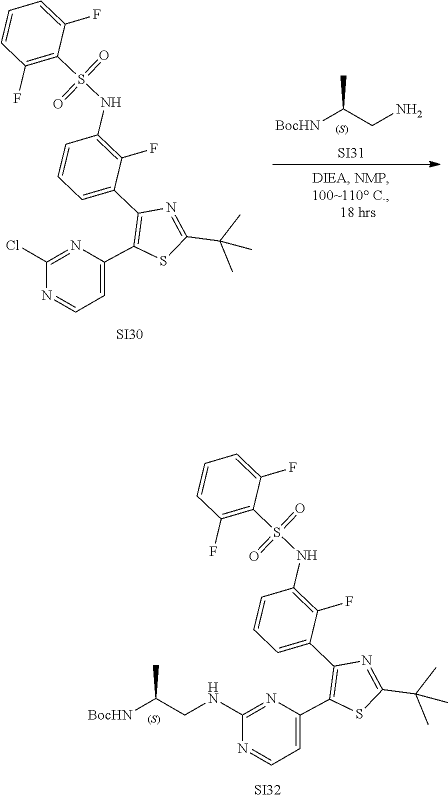

To a solution of compound SI30 (3.70 g, 6.86 mmol) and SI31 (1.20 g, 6.86 mmol) in NMP (20.0 mL) was added DIEA (2.66 g, 20.5 mmol, 3.59 mL). The mixture was stirred at 100-110° C. under N₂ for 18 hrs. LCMS (EC2025-12-P1C) showed reactant was consumed completely and compound of the desired mass (M+1=677.2, RT=0.590 min) was detected. The reaction was cooled at 20˜25° C. The reaction solution was diluted with water (40.0 mL) and extracted with ethyl acetate (120 mL (40.0 mL*3). The combined organic layers were washed with brine (120 mL (40.0 mL*2), dried over Na₂SO₄, filtered, and concentrated under reduced pressure to give a residue. The res=0.590 min) was detected. The reaction was cooled at 20≠25° C. The reaction solution was diluted with water (40.0 mL) and extracted with ethyl acetate (120 mL (40.0 mL*3). The combined organic layers were washed with brine (120 mL (Petroleum ether:Ethyl acetate=2:1 R1: Rf=0.24; P1: Rf=0.29) and were purified by flash silica gel chromatography (ISCO®; Sepa Flash® Silica Flash Column, Eluent of 0˜50% Ethyl acetate/Petroleum ether gradient). The solution was concentrated under reduced pressure to give compound SI32 (3.50 g, 5.17 mmol) as a brown oil.

¹H NMR: EC2025-12-P1, 400 MHz, CDCl₃δ 7.93 (t, J=5.8 Hz, 1H), 7.58-7.42 (m, 1H), 7.36-7.27 (m, 1H), 7.27-7.19 (m, 1H), 7.17-7.10 (m, 1H), 6.94 (br t, J=8.8 Hz, 2H), 6.54 (br d, J=8.6 Hz, 1H), 6.27 (dd, J=8.2, 10.8 Hz, 1H), 3.93-3.75 (m, 2H), 3.31 (br s, 1H), 3.23-3.16 (m, 1H), 3.13 (br d, J=5.8 Hz, 1H), 1.45 (s, 9H), 1.38 (s, 9H), 1.20-1.17 (m, 3H). LCMS: EC2025-12-P1C, product: RT=0.590 min, m/z=677.2 (M+H)⁺

Step 5. Preparation of Compound SI33, (S)-N-(3-(5-(24(2-aminopropyl)amino)pyrimidin-4-y1)-2-(tert-butyl)thiazol-4-yl)-2-fluorophenyl)-2,6-difluorobenzenesulfonamide

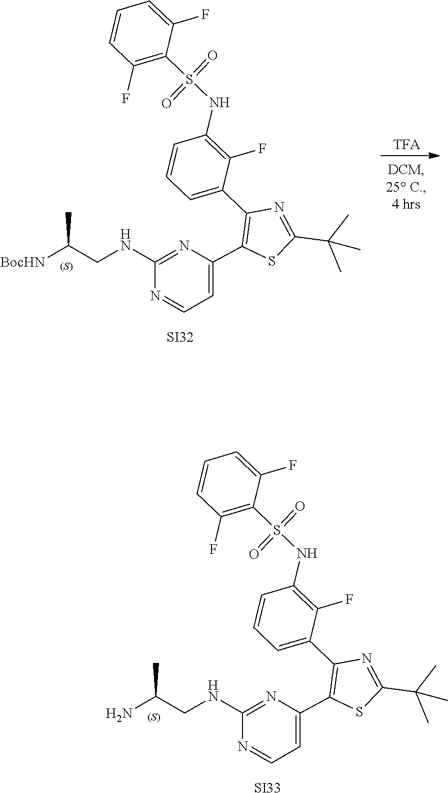

A mixture of compound SI32 (3.30 g, 4.88 mmol, 1 eq) in DCM (10.0 mL) and TFA (3.00 mL) was degassed and purged with N₂ 3 times. The mixture was then stirred at 25° C. for 4 hrs. under N₂ atmosphere. LC-MS (EC1797-41-P1B2) and HPLC (EC1797-41-P1A4) showed Reactant was consumed completely and one main peak with desired mass was detected. The reaction mixture concentrated under reduced pressure to give a residue. The residue was purified by prep-HPLC (HCl condition;). The compound SI33 (0.30 g, 520.2 μmol) was obtained as a yellow solid.

LCMS: EC2025-12-P1C, product: RT=0.478 min, m/z=576.9 (M+H)⁺.

Step 6. Preparation of Compound SI35, (S)-N-(1-((4-(2-(tert-butyl)-4-(3-((2,6-difluorophenyl)sulfonamido)-2-fluorophenyl)thiazol-5-yl)pyrimidin-2-yl)amino)propan-2-yl)-3-((tert-butyldimethylsilyl)oxy)propanamide

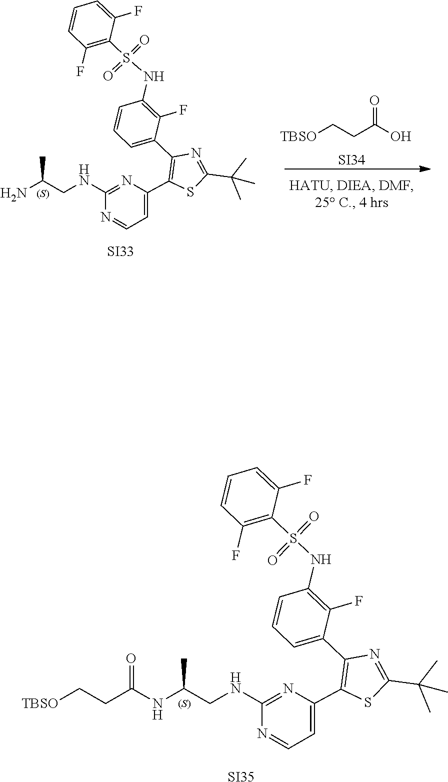

To a solution of compound SI34 (35.4 mg, 173 μmol, 1.00 eq) and compound SI33 (100 mg, 173 μmol, 1.00 eq) in DMF (4 mL) was added HATU (98.91 mg, 260.12 μmol, 1.50 eq) and DIEA (44.8 mg, 347 μmol, 60.4 uL, 2.00 eq), and the mixture was stirred at 25° C. for 3 hrs. LCMS (EC2393-5-P1L) showed Reactant was consumed completely and one main peak with desired m/z or desired mass was detected. The reaction mixture was diluted with water (40 mL) and extracted with Ethyl acetate (80 mL, 40.0 mL*2). The combined organic layers were washed with brine (40 mL), dried over Na₂SO₄, filtered, and concentrated under reduced pressure to give a residue. Compound SI35 (80 mg, 105 μmol, 60.5% yield) was obtained as a yellow oil.

LCMS: EC2393-5-P1L, product: RT=0.541 min, m/z=763.2 (M+H)⁺.

Step 7. Preparation of Compound K6, (S)-N-(1-((4-(2-(tert-butyl)-4-(3-(2,6-difluorophenyl)sulfonamido)-2-fluorophenyl)thiazol-5-yl)pyrimidin-2-yl)amino)propan-2-yl)-3-hydroxypropanamide

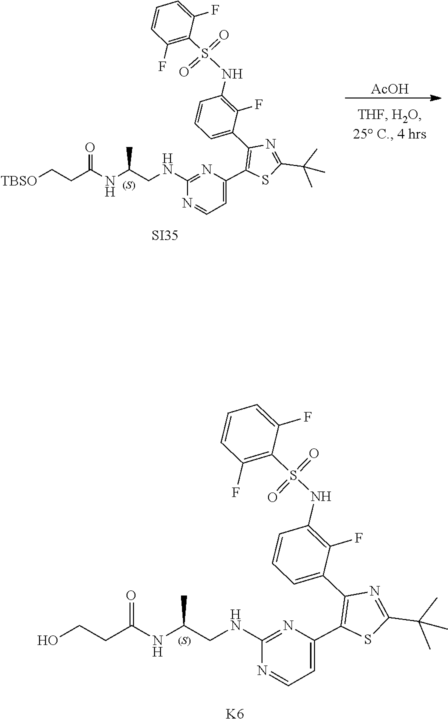

To a solution of compound SI35 (0.10 g, 131 μmol, 1.00 eq) in THF (0.5 mL), and H₂O (0.5 mL) was added AcOH (157 mg, 2.62 mmol, 145 uL, 20.0 eq), then the mixture was stirred at 25° C. for 2 hrs. LCMS (EC2393-6-P1L1) showed Reactant was consumed completely and one main peak with desired m/z or desired mass was detected. The reaction mixture was filtered and concentrated under reduced pressure to give a residue. The residue was purified by prep-HPLC (FA condition; column: Phenomenex Luna C18 150*25mm*10 um; mobile phase: [water (FA)-ACN]; B %: 35%-65%, 10 min). Compound K6 (60 mg, 91.6 μmol, 69.8% yield, 99.0% purity) was obtained as a white solid. 30.26 mg of K6 was delivered.

¹H NMR: EC2393-6-P1A, 400 MHz, DMSO-d₆ δ 14.10-14.11 (m, 1 H) 10.90 (s, 1 H) 8.08-8.08 (m, 1 H) 8.07 (d, J=5.13 Hz, 1 H) 7.67-7.74 (m, 2 H) 7.36-7.50 (m, 2 H) 7.17-7.35 (m, 5 H) 5.82-6.08 (m, 1 H) 4.59 (t, J=5.19 Hz, 1 H) 3.95-4.07 (m, 1 H) 3.59-3.67 (m, 2 H) 2.26 (t, J=6.57 Hz, 3 H) 1.45 (s, 9 H) 1.07 (br d, J=6.63 Hz, 3 H) LCMS: EC2393-6-P1C1, product: RT=0.436 min, m/z=649.3 (M+H)⁺. HPLC: EC2393-6-P1H_{1,} product: RT=2.055 min, 99.3% purity.

### Example 7. Preparation of Compound K8

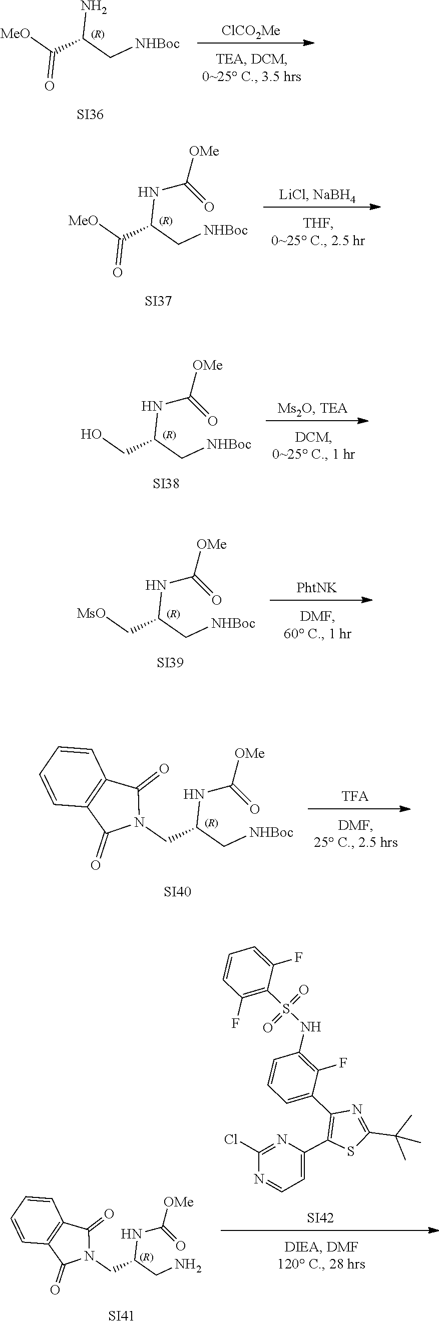

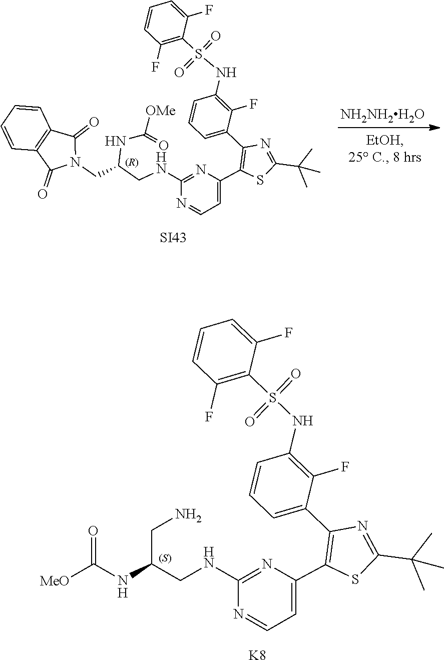

Step 1. Preparation of Compound SI37, Methyl (R)-3-((tert-butoxycarbonyl)amino)-2-((methoxycarbonyl)amino)propanoate

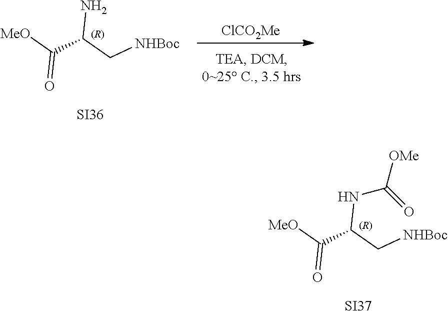

To a solution of compound SI36 (5.00 g, 22.9 mmol) and TEA (3.64 g, 35.9 mmol, 5.00 mL) in DCM (50.0 mL) at 0° C. under N₂ was added methyl chloroformate (2.41 g, 25.5 mmol, 1.98 mL), the mixture was stirred at 25° C. for 3.5 hrs. TLC (Petroleum ether:Ethyl acetate=1:1 R1: Rf=0.24; P1: Rf=0.43) indicated Reactant was consumed completely and one new spot formed. The reaction was clean according to TLC. The reaction was quenched with sat. NaHCO₃ (50.0 mL), then extract with DCM (20.0 mL*2), the organic phase was dried over Na₂SO₄, filtered, concentrated to give a residue. The residue (Petroleum ether:Ethyl acetate=1:1 R1: R_{f}=0.24; P1:R_{f}=0.43) was purified by column chromatography (SiO₂, Petroleum ether/Ethyl acetate=5/1 to 1/1). The solution was concentrated to give compound SI37 (3.21 g, 11.6 mmol) as white oil.

¹H NMR: EC2025-36-P1A1, 400 MHz, CDCl_{3, 8 5.73} (br s, 1H), 4.88 (br s, 1H), 4.32 (br d, J=3.9 Hz, 1H), 3.70 (s, 3H), 3.62 (s, 3H), 3.47 (br s, 2H), 1.36 (s, 9H).

Step 2. General procedure for preparation of compound SI38, tert-butyl methyl (3-hydroxypropane-1,2-diyl)(R)-dicarbamate

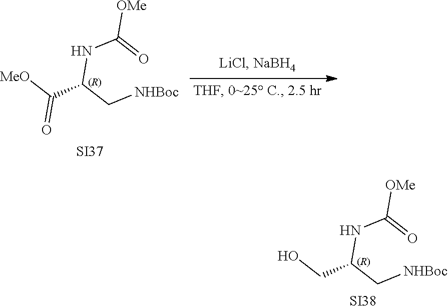

To a solution of compound SI37 (3.21 g, 11.6 mmol) in THF (60.0 mL) at 0° C. was added NaBH₄ (0.36 g, 9.52 mmol) and LiCl (541 mg, 12.7 mmol). The mixture was stirred at 25° C. for 2.5 hr. TLC (Petroleum ether:Ethyl acetate=1:1 R1:R_{f}=0.57; P1: R_{f}=0.25) indicated Reactant remained, and one major new spot with larger polarity was detected. The reaction mixture was quenched by addition 1M HCl (50.0 mL) at 25° C., and extracted with EtOAc (100 mL, 50.0 mL*2). The combined organic layers were washed with brine (100 mL, 50.0 mL*2), dried over Na₂SO₄, filtered, and concentrated under reduced pressure to give a residue. The residue was purified by column chromatography (SiO₂, Petroleum ether/Ethyl acetate=10/1 to 0/1). The solution was concentrated under reduced pressure to give compound SI38 (2.36 g, 9.51 mmol) as white solid.

¹H NMR: EC2025-41-P1A, 400 MHz, CDCl₃δ5.34 (br d, J=5.3 Hz, 1H), 5.00 (br s, 1H), 3.73 (br d, J=1.8 Hz, 1H), 3.69 (s, 3H), 3.64 (br s, 2H), 3.55 (br d, J=11.1 Hz, 1H), 3.40-3.30 (m, 1H), 3.28-3.20 (m, 1H), 1.46 (s, 9H).

Step 3. Preparation of Compound SI39, (R)-3-((tert-butoxycarbonyl)amino)-2-((methoxycarbonyl)amino)propyl methanesulfonate

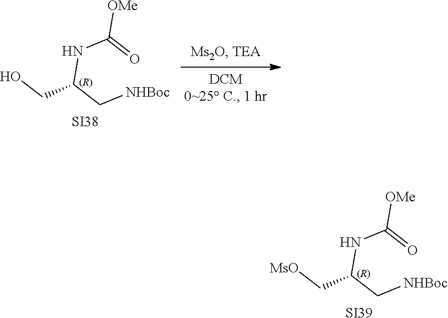

To a solution of compound SI38 (1.00 g, 4.03 mmol) and TEA (815 mg, 8.06 mmol, 1.12 mL) in DCM (10.0 mL) was added methyl sulfonyl methane sulfonate (1.05 g, 6.04 mmol) at 0° C. The reaction mixture was stirred at 25° C. for 1 hr. TLC (Petroleum ether: Ethyl acetate=1:1 R1: Rf=0.23; P1: Rf=0.34) indicated Reactant was consumed completely and one new spot formed. The reaction was clean according to TLC. The reaction mixture was quenched by addition water (30.0 mL) at 25° C., and extracted with DCM (60.0 mL, 30.0 mL*2). The combined organic layers were washed with brine (60.0 mL, 30.0 m*2), dried over Na₂SO₄, filtered, and concentrated under reduced pressure to give a residue. The residue was purified by column chromatography (SiO₂, Petroleum ether/Ethyl acetate=6/1 to 0/1). The solution was concentrated under reduced pressure to give a compound SI39 (1.50 g, crude) as white solid.

Step 4. Preparation of Compound SI40, tert-butyl methyl (3-(1,3-dioxoisoindolin-2-yl)propane-1,2-diyl)(R)-dicarbamate

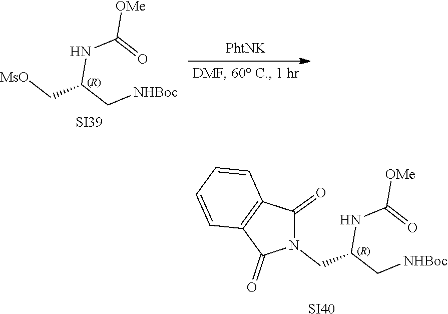

To a solution of compound SI39 (1.50 g, 4.60 mmol) in DMF (15.0 mL) was added potassium 1, 3-dioxoisoindolin-2-ide (1.28 g, 6.90 mmol). The mixture was stirred at 60° C. for 1 hr. The LCMS (EC2025-47-P1A1) shows reactant was consumed. TLC (Petroleum ether:Ethyl acetate=1:1 R1: Rf=0.34; P1: Rf=0.43) indicated reactant was consumed completely and one new spot formed. The reaction was clean according to TLC. The reaction was cooled at 25° C. The reaction mixture was partitioned between water (30.0 mL) and EtOAc (30 mL*2). The organic phase was separated, washed with brine (60.0 mL, 30.0 mL*2), dried over Na₂SO₄, filtered, and concentrated under reduced pressure to give a residue. The residue was purified by column chromatography (SiO₂, Petroleum ether/Ethyl acetate=5/1 to 0/1). The solution was concentrated under reduced pressure to give compound SI40 (1.50 g, 3.97 mmol) as white solid.

LCMS: EC2025-47-P1A1, product: RT=0.380 min, m/z=278.1 (M-100).

¹H NMR: EC2025-47-P1A, 400 MHz, CDCl₃. δ 7.82-7.76 (m, 2H), 7.71-7.64 (m, 2H), 5.41 (br s, 1H), 5.04 (br s, 1H), 3.96-3.85 (m, 1H), 3.80-3.70 (m, 2H), 3.53 (s, 3H), 3.30-3.13 (m, 2H), 1.38 (s, 9H).

Step 5. Preparation of Compound SI41, Methyl (R)-(1-amino-3-(1,3-dioxoisoindolin-2-yl)propan-2-yl)carbamate

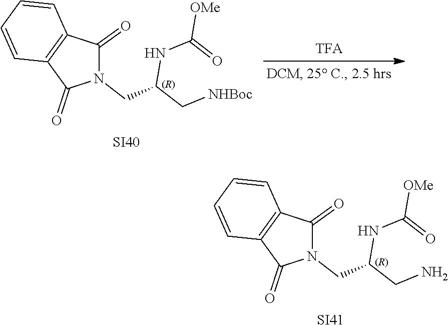

To a solution of compound SI40 (1.10 g, 2.91 mmol, 1.00 eq) in DCM (3.00 mL) was added TFA (3.08 g, 27.0 mmol, 2.00 mL, 9.27 eq) at 25° C. The mixture was stirred at 25° C. for 2.5 hrs. LC-MS (EC2025-64-P1A1) showed Reactant was consumed completely and desired mass was detected. The reaction solution was concentrated under reduced pressure to give a residue. The crude product was triturated with MTBE 10.0 mL at 25° C. for 5 min. The compound SI41 (1.05 g, 2.62 mmol, 89.9% yield, 97.7% purity, TFA) as a white solid.

LCMS: EC2025-64-P1C1, product: RT=0.281 min, m/z=278.1 (M+H)⁺.

Step 6. preparation of compound SI43

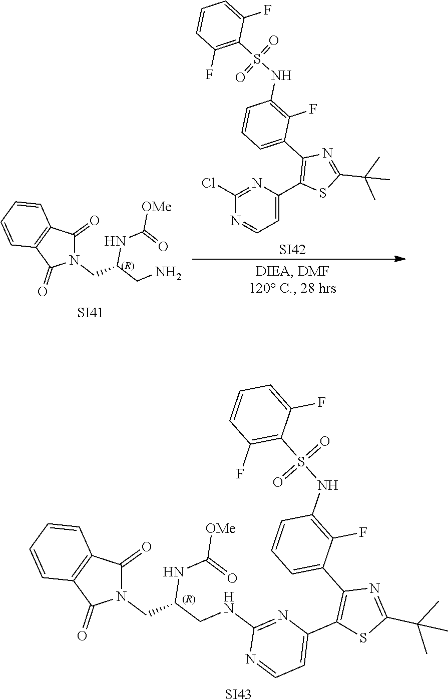

A mixture of compound SI42 (600 mg, 1.11 mmol, 1.00 eq) , compound SI41 (668 mg, 1.67 mmol, 97.7% purity, 1.50 eq, TFA), DIEA (431 mg, 3.34 mmol, 581 μIL, 3.00 eq) in NMP (2.00 mL) was degassed and purged with N₂ for 3 times, and then the mixture was stirred at 120° C. for 28 hr. under N₂ atmosphere. LC-MS (EC2025-66-P1A3) showed 13.5% of Reactant remained. Several new peaks were shown on LC-MS and 4.6% of desired compound was detected. The reaction solution was filtered. The filter liquor was purified by reversed-phase HPLC (column: YMC Triart C18 250*50mm*7 um; mobile phase: [water (FA)-ACN]; B %: 58%-88%, 10 min) Compound SI43 (50 mg, 64.1 μmol, 5.76% yield) was obtained as a yellow solid.

LCMS: EC2025-66-P1A3, product: RT=0.581 min, m/z =780 (M+H)⁺.

### Step 7. Preparation of Compound K8

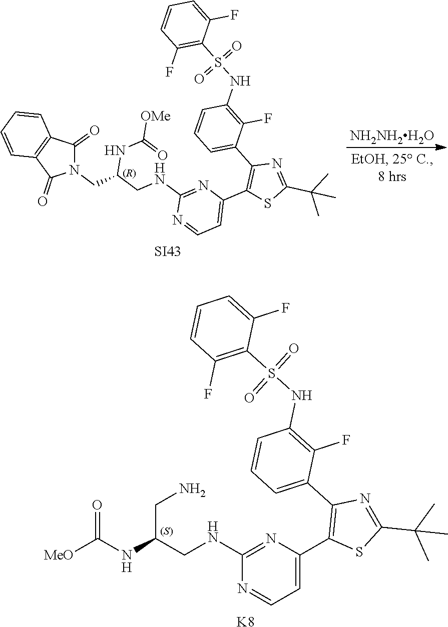

To a solution of compound 7 (50 mg, 64.1 μmol, 1.00 eq) in EtOH (1.00 mL) was added N₂H₄.H₂O (0.10 g, 1.96 mmol, 97.0 μL, 98% purity, 30.5 eq). The mixture was stirred at 25° C. for 8 hr. LCMS showed Reactant was consumed completely and desired mass was detected. The reaction mixture was quenched by addition water (10.0 mL) at 25° C., and extracted with EtOAc (40.0 mL, 20.0 mL*2). The combined organic layers were dried over Na₂SO₄, filtered, and concentrated under reduced pressure to give a residue. The residue was purified by reversed-phase HPLC (column Welch Xtimate C18 150*25 mm*5 um mobile phase: [water (HCl)-ACN]; B %: 18%-48%, 8mM). Compound K8 (9.14 mg, 12.7 μmol, 19.8% yield, 95.5% purity, HCl) was obtained as a yellow solid.

¹H NMR: EC2025-70-P1A1, 400 MHz, DMSO-d₆.δ 10.87 (s, 1H), 8.08 (d, J=5.1 Hz, 1H), 7.90 (br s, 2H), 7.74-7.64 (m, 1H), 7.55-7.18 (m, 7H), 6.05-5.89 (m, 1H), 3.96-3.87 (m, 1H), 3.57 (s, 3H), 3.35-3.22 (m, 2H), 3.02-2.89 (m, 1H), 2.81 (br dd, J=4.6, 8.3 Hz, 1H), 1.42 (s, 9H). LCMS: EC2025-70-P1C3, product: RT=1.673 min, m/z=650.1 (M+H)⁺. HPLC: EC2025-70-P1H2, product: RT=2.335 min, purity=95.54%. SFC: EC2025-70-P1A_d4, product: RT=2.031 min, ee % =100%.

### Example 8. Preparation of Compound K10

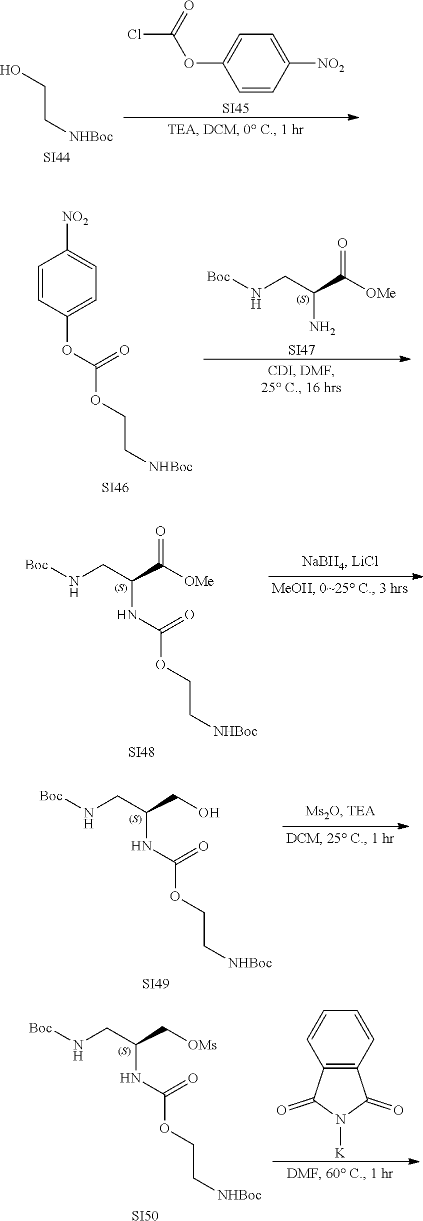

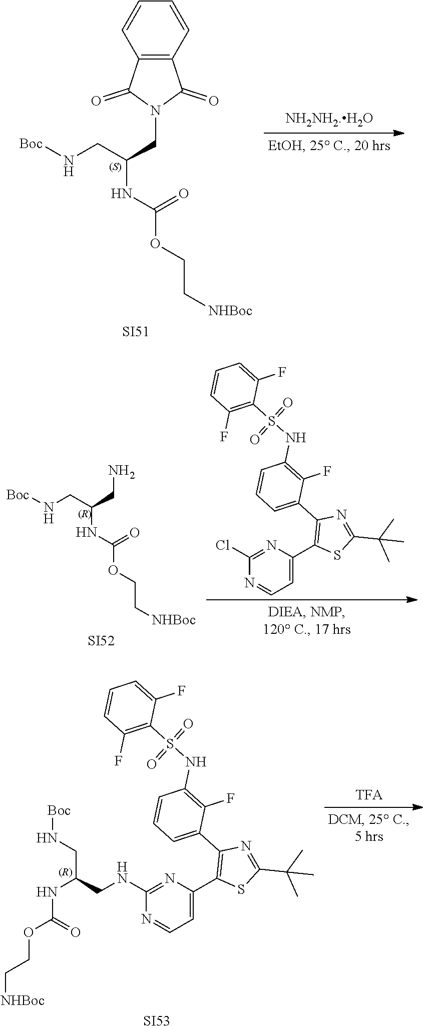

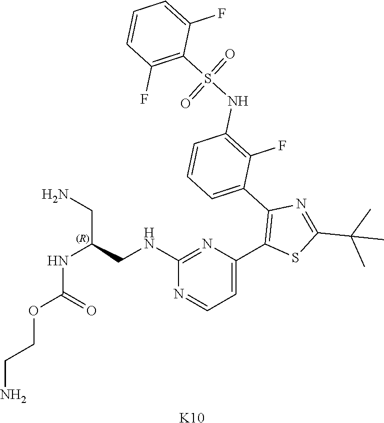

Step 1. Preparation of Compound SI46, Tert-butyl (2-(((4-nitrophenoxy)carbonyl)oxy)ethyl)carbamate

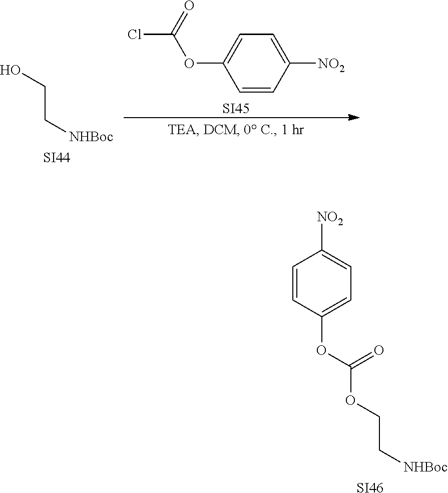

To a solution of compound SI44 (10.0 g, 62.0 mmol, 9.62 mL, 1.00 eq) and (4-nitrophenyl) carbonchloridate, SI45, (13.7 g, 68.2 mmol, 1.10 eq) in DCM (100 mL) was added TEA (15.6 g, 155 mmol, 21.5 mL, 2.50 eq) at 0° C. The mixture was stirred at 0° C. for 1 hr. TLC (Petroleum ether:Ethyl acetate=5:1 R1: R_{f}=0.24; P1: R_{f}=0.43) indicated Reactant was consumed completely and one new spot formed. The reaction mixture was quenched by addition of water (100 mL) at 25° C., and extracted with DCM (100 mL, 50.0 mL*2). The combined organic layers were dried over Na₂SO₄, filtered, and concentrated under reduced pressure to give a residue. The residue was not purified and was directly used as the next step. Compound SI46 (23.3 g, 39.9 mmol, 64.5% yield, 56.0% purity) was obtained as a yellow oil.

LCMS: EC2025-58-P1C2, product: RT=0.570 min, m/z=349.0 (M+Na)⁺.

Step 2. Preparation of Compound SI48, methyl (S)-2-(((tert-butoxycarbonyl)amino)methyl)-11,11-dimethyl-4,9-dioxo-5,10-dioxa-3,8-diazadodecanoate

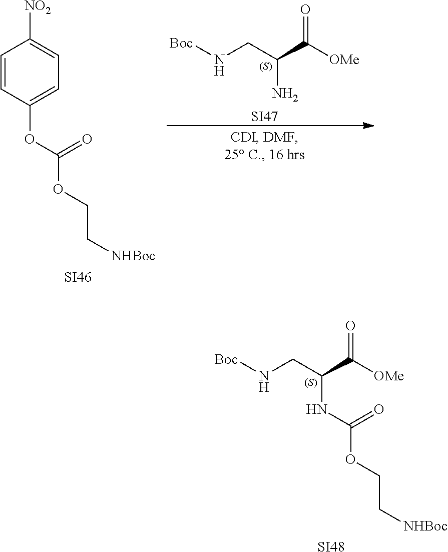

To a solution of compound SI47 (5.00 g, 22.9 mmol, 1.00 eq) and compound SI46 (16.0 g, 27.4 mmol, 56% purity, 1.20 eq) in DCM (50.0 mL) was added TEA (3.48 g, 34.3 mmol, 4.78 mL, 1.50 eq). The mixture was stirred at 25° C. for 16 hr. TLC (Petroleum ether: Ethyl acetate=2:1 R1: R_{f}=0.02; P1: R_{f}=0.20) indicated Reactant was consumed completely and one new spot formed. The reaction was clean according to TLC. The reaction solution was concentrated under reduced pressure to give a residue. The residue was purified by column chromatography (SiO₂, Petroleum ether/Ethyl acetate=10/1 to 2/1). Compound SI48 (6.30 g, 15.5 mmol, 67.3% yield) was obtained as a yellow oil.

¹H NMR: EC2025-60-P1A, 400 MHz, CDCl₃. δ 5.80 (br s, 1H), 4.99 (br s, 2H), 4.40 (br s, 1H), 4.16-4.11 (m, 2H), 3.79 (s, 3H), 3.61-3.55 (m, 2H), 3.44-3.35 (m, 2H), 1.48-1.45 (m, 18H).

Step 3. Preparation of Compound SI49, 2-((tert-butoxycarbonyl)amino)ethyl tert-butyl (3-hydroxypropane-1,2-diyl)(S)-dicarbamate

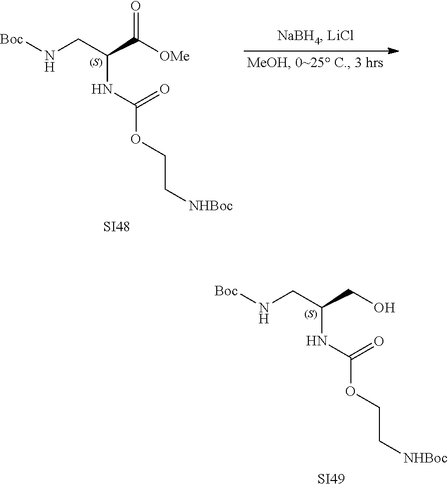

To a solution of compound SI48 (2.50 g, 6.17 mmol, 1.00 eq) in EtOH (40.0 mL) was added LiCl (287 mg, 6.78 mmol, 138 μL, 1.10 eq) and NaBH₄ (0.45 g, 11.9 mmol, 1.93 eq) at 0° C. The mixture was stirred at 25° C. for 3 h. TLC (Petroleum ether:Ethyl acetate=1:1 R1: R_{f}=0.24; P1: R_{f}=0.15) indicated Reactant was consumed completely and one new spot formed. The reaction mixture was quenched by addition 1M HCl, (80.0 mL) at 25° C., and extracted with EtOAc (100 mL, 50.0 mL*2). The combined organic layers were washed with brine (100 mL, 50.0 mL*2), dried over Na₂SO₄, filtered, and concentrated under reduced pressure to give a residue. The residue was purified by column chromatography (SiO₂, Petroleum ether/Ethyl acetate=10/1 to 2/1). The compound SI49 (2.95 g, crude) as a yellow gum.

Step 4. Preparation of Compound SI50, (S)-2-4(tert-butoxycarbonyl)amino)methyl)-11,11-dimethyl-4,9-dioxo-5,10-dioxa-3,8-diazadodecyl methane sulfonate.

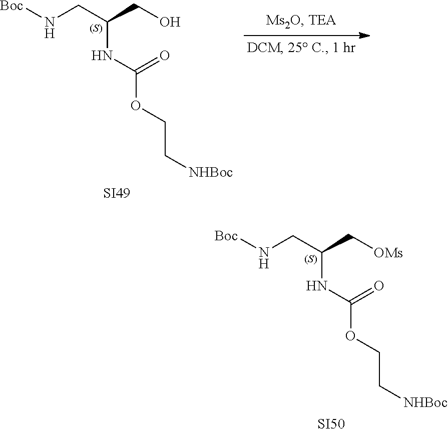

To a solution of compound SI49 (1.00 g, 2.65 mmol, 1.00 eq) in DCM (10.0 mL) was added methylsulfonyl methanesulfonate (692 mg, 3.97 mmol, 1.50 eq) and TEA (536 mg, 5.30 mmol, 737 uL, 2.00 eq). The mixture was stirred at 25° C. for 1 hr. TLC (Petroleum ether:Ethyl acetate=1:1 R1: R_{f}=0.15; P1: R_{f}=0.21) indicated Reactant was consumed completely and one new spot formed. The reaction mixture was quenched by addition water (20 mL) at 25° C., and extracted with DCM (50 mL, 25 mL*2). The combined organic layers were washed with brine (50 mL, 25 mL*2), dried over Na₂SO₄, filtered, and concentrated under reduced pressure to give a residue. Compound SI50 (1.09 g, crude) was obtained as a yellow gum.

Step 5. Preparation of Compound SI51, 2-((tert-butoxycarbonyl)amino)ethyl tert-butyl (3-(1,3-dioxoisoindolin-2-yl)propane-1,2-diyl)(S)-dicarbamate

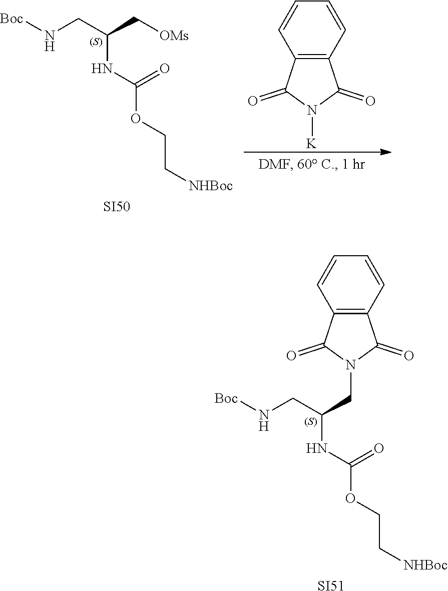

To a solution of compound SI50 (1.09 g, 2.39 mmol, 1.00 eq) in DMF (15.0 mL) was added (1,3-dioxoisoindolin-2-yl)potassium (665 mg, 3.59 mmol, 1.50 eq). The mixture was stirred at 60° C. for 1 hr. LC-MS (EC2025-67-P1A1) showed Reactant was consumed completely and desired mass was detected. The reaction mixture was quenched by addition water 30 mL at 25° C., and extracted with EtOAc (50 mL, 25 mL*2). The combined organic layers were washed with brine (50 mL, 25 mL*2), dried over Na₂SO₄, filtered, and concentrated under reduced pressure to give a residue. The residue (Petroleum ether:Ethyl acetate=1:1 R1: R_{f}=0.21; P1: R_{f}=0.43) was purified by column chromatography (SiO2, Petroleum ether/Ethyl acetate=10/1 to 1/2). Compound SI51 (852 mg, 1.40 mmol, 58.6% yield, 83.4% purity) was obtained as a yellow solid.

LCMS: EC2025-67-P1XD, product: RT=0.522 min, m/z=407.2 (M+H)³⁰ .

Step 6. Preparation of Compound SI52, 2-((tert-butoxycarbonyl)amino)ethyl tert-butyl (3-aminopropane-1,2-diyl)(R)-dicarbamate

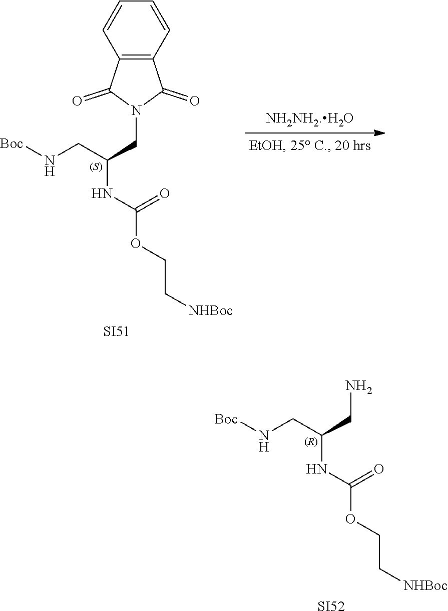

To a solution of compound SI51 (852 mg, 1.40 mmol, 83.4% purity, 1.00 eq) in EtOH (10.0 mL) was added N₂H₄.H₂O(0.35 g, 6.85 mmol, 339 μL, 98% purity, 4.88 eq). The mixture was stirred at 25° C. for 20 hr. LC-MS(EC2025-68-P1A3) showed Reactant was consumed completely. The reaction mixture was quenched by addition water (10.0 mL) at 25° C., and extracted with EtOAc (40.0 mL, 20 mL*2). The combined organic layers were dried over Na₂SO₄, filtered, and concentrated under reduced pressure to give a residue. The residue was charged EtOH (20 mL) and stirred for 5 min and filtered. The filter liquor was concentrated to give a crude product. Compound SI52 (656 mg, crude) as a yellow solid.

LCMS: EC2025-67-P1XD, product: RT=0.184 min, m/z=322.7 (M-56+H)⁺.

Step 7. Preparation of Compound SI53, 2-((tert-butoxycarbonyl)amino)ethyl tert-butyl (34(4-(2-(tert-butyl)-4-(34(2,6-difluorophenyl)sulfonamido)-2-fluorophenyl)thiazol-5-yl)pyrimidin-2-yl)amino)propane-1,2-diyl)(R)-dicarbamate

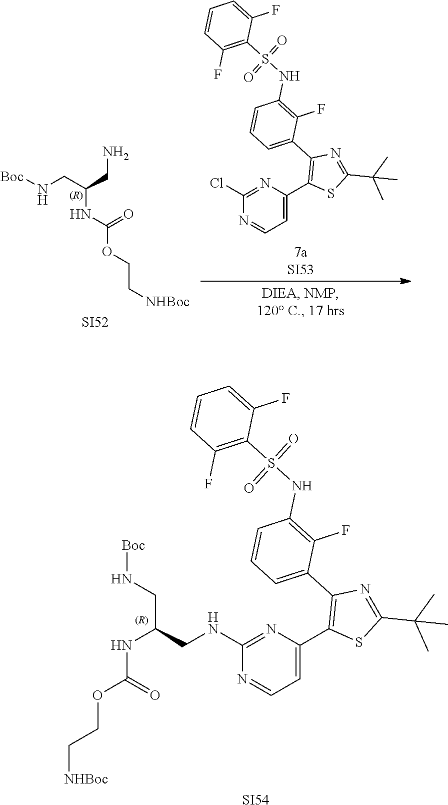

To a solution of compound SI52 (528 mg, 1.40 mmol, 1.00 eq) and compound SI53 (755 mg, 1.40 mmol, 1 eq) in NMP (3.00 mL) was added DIEA (543 mg, 4.21 mmol, 732 μL, 3.00 eq). The mixture was stirred at 120° C. for 17 hr. LC-MS (EC2025-72-P1A1) showed 17.5% of Reactant remained. Several new peaks were shown on LC-MS and 14.6% of desired compound was detected. The reaction solution was filtered. The filter liquor was purified by reversed-phase HPLC (column Phenomenex C18 150*25mm*10 um; mobile phase: [water (NH₃H₂O)-ACN]; B %: 35%-65%, 10 min). Compound SI54 (165 mg, 187.72 μmol, 13.38% yield) as a yellow brown solid.

LCMS: EC2025-67-P1XD, product: RT=0.568 min, m/z =879.2 (M+H)⁺.

Step 8. Preparation of Compound K10, 2-aminoethyl (S)-(1-amino-3((4-(2-(tert-butyl)-4-(3-((2,6-difluorophenyl)sulfonamido)-2-fluorophenyl)thiazol-5-yl)pyrimidin-2-yl)amino)propan-2-yl)carbamate compound with 2,2,2-

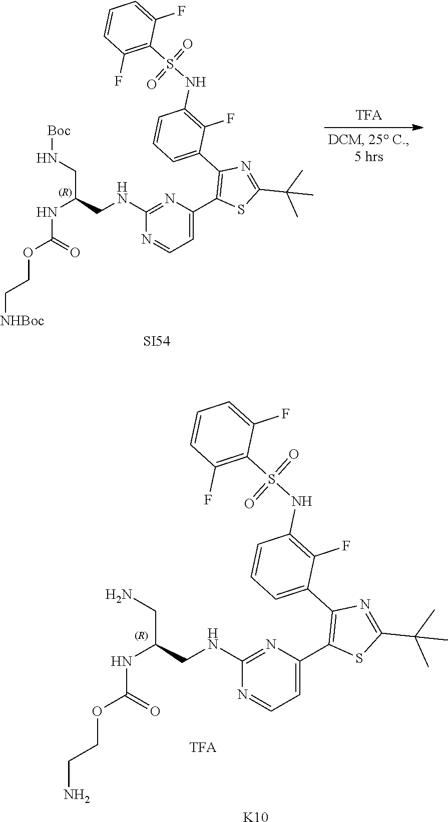

To a solution of compound SI54 (150 mg, 170 μmol, 1.00 eq) in DCM (2.00 mL) was added TFA (77 mg, 682 μmol, 50 uL, 4.00 eq).The mixture was stirred at 25° C. for 5 hrs. LC-MS (EC2025-75-P1A4) showed Reactant was consumed completely and desired mass was detected. The reaction solution was filtered. The filter liquor was purified by reversed-phase HPLC (column Welch Xtimate C18 150*25 mm*5 um; mobile phase: [water (FA)-ACN]; B %: 5%-35%, 10 min). The compound K10 (77.47 mg, 93.30 μmol, 54.67% yield, 95.48% purity, TFA) as a yellow solid.

¹HNMR: EC2025-75-P1A2, 400 MHz, MeOD. δ 8.49 (br s, 1H), 8.12 (d, J=5.3 Hz, 1H), 7.67-7.38 (m, 2H), 7.20-7.12 (m, 1H), 7.07 (br t, J=8.9 Hz, 2H), 6.54-6.29 (m, 1H), 4.39-4.23 (m, 2H), 4.09-3.97 (m, 1H), 3.44-3.34 (m, 2H), 3.25-3.18 (m, 2H), 3.16-3.07 (m, 1H), 3.00-2.88 (m, 1H), 1.49 (s, 9H). LCMS: EC2025-75-P1C2, product: RT=1.465 min, m/z=679.1 (M+H)⁺. HPLC: EC2025-75-P1H₂, product: RT=1.880 min, purity=95.48%. SFC: EC2025-75-P1A_d7, product: RT=0.268 min, ee % =100%.

1. A compound of Formula I 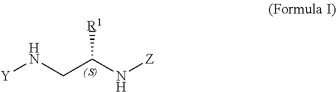
or a pharmaceutically acceptable salt thereof, wherein
R¹ is hydrogen, —Cₙ, —NHCₙ, or —CₙC═NH, where Cₙ is an alkyl or alkenyl group having the indicated number of carbon atoms and the requisite number of hydrogen atoms, and n is an integer from 1 to 6;
Y is 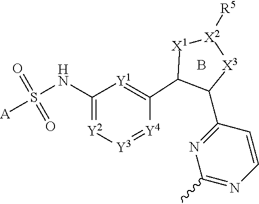
A is Ring A, which is C₃-C₇cycloalkyl, phenyl, or a 5-6-membered heterocycle having 1 or 2 heteroatoms independently selected from N, O, and S, each of which Ring A is optionally substituted; or
A is a mono- or di-(C₁-C₆alkyl)amino;
Ring B is a 5-membered unsaturated or aromatic heterocyclic ring, with at least one heteroatom;
X¹ is N or C;
X² is S or C;
X³ is S, O, or N;
Y¹, Y², Y³, and Y⁴ are independently N or CR⁶, where 0 or 1 of Y¹, Y², Y³, and Y⁴ are N;
Z is 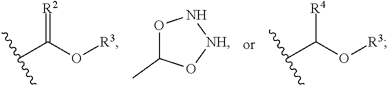
R² is oxygen, Cₙ, ═CₙNH₂, ═CnOH, ═NCₙNH₂, or ═NCₙOH;
R³ is —Cₙ, —CₙOH, or —CₙNH₂; and
R⁴ is —CₙOH, —Cₙ═NH, or —CₙNH₂;
R⁵ is hydrogen, halogen, cyano, hydroxyl, amino, oxo, —CHO, —SO₂, C₃-C₆cycloalkyl, C₃-C₅heterocycloalkyl, or C₁-C₆alkyl in which one carbon atom may be replaced by O, S, or NR⁷ and which C₁-C₆alkyl is optionally substituted with one or more substituents independently chosen from halogen, hydroxyl, amino, oxo, and —COOH;
R⁶ is independently chosen at each occurrence from hydrogen, halogen, hydroxyl, C₁-C₆alkyl, C₁-C₆alkoxy, C₃-C₇cycloalkyl, C₃-C₇cycloalkoxy, —C(O)C₁-C₆alkyl, —C(O)C₃-C₇cycloalkyl, C₁-C₂haloalkyl, and C₁-C₂haloalkoxy; and
R⁷ is independently chosen at each occurrence from hydrogen and C₁-C₆alkyl.

2. (canceled)

3. The Formula (I) compound of claim 1, or salt thereof, Y having the structure 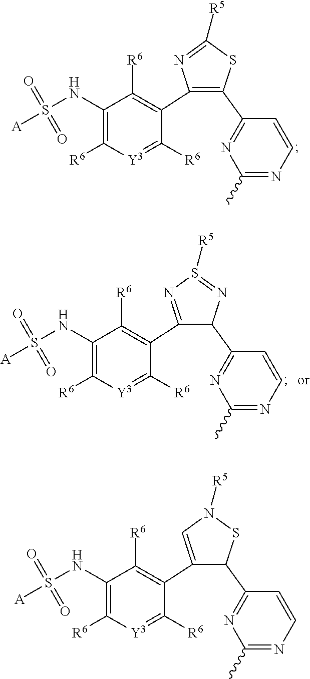
wherein
A is Ring A, a phenyl which is unsubstituted or substituted with one or more substituents independently chosen from halogen, hydroxyl, cyano, amino, C₃-C₆cycloalkyl, a in which one carbon atom may be replaced by O, S, or NR⁷ and which C₁-C₆alkyl is optionally substituted with one or more substituents independently chosen from halogen, hydroxyl, amino, oxo, and —COOH; and
Y¹, Y², Y³, and Y⁴ are all CR⁶.

4. (canceled)

5. The compound or salt of claim 3,
wherein
A is Ring A, a phenyl which is substituted with one or more halogen substituents; and
Y¹, Y², Y³, nd Y⁴ are all CR⁶ and R⁶ is independently chosen at each occurrence from hydrogen and halogen.

6-7. (canceled)

8. The compound or salt of claim 1, wherein Y¹ is CR⁶ and R⁶ is F, Cl, Br, or methyl, Y³ is nitrogen, and Y² and Y⁴ are CH.

9. The compound or salt of claim 1, wherein Y¹ is CR⁶ and R⁶ is F, Cl, Br, or methyl, and Y², Y³, and Y⁴ are CH.

10-11. (canceled)

12. The compound or salt of claim 1, wherein Y is

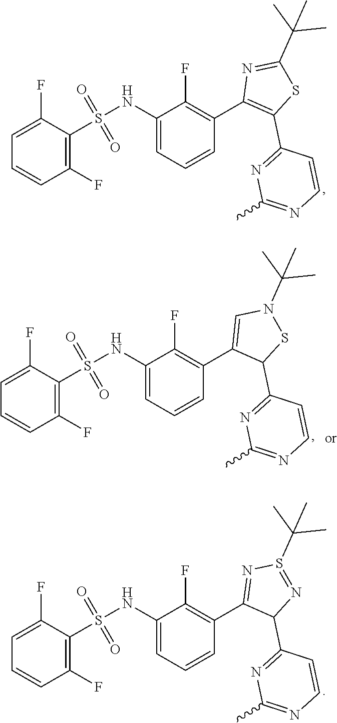

13. The compound or salt of claim 3, wherein R¹ is methyl, ethyl, —CH₂NH₂, —CH₂CHNH, or —NHCH₃.

14. The compound or salt of claim 13, wherein
Z is 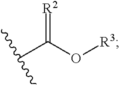
and
R² is oxygen, ═CdₙNH₂, ═C,OH, ═NCₙNH₂, or ═NCₙOH, where n is 1, 2, 3, or 4.

15. The compound or salt of claim 13, wherein
Z is 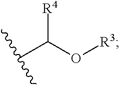
and R⁴ is —CₙOH, —Cₙ═NH, or —CₙTH₂; where n is 1, 2, 3, or 4.

16. The compound or salt of claim 13, wherein R³ is —Cₙ, —CₙOH, or
—CₙNH₂; where n is 1 or 2.

17. The compound or salt of claim 13, wherein
Z is

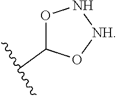 [SMILES: *C1ONNO1]

18. The compound or salt of claim 1, wherein the compound is

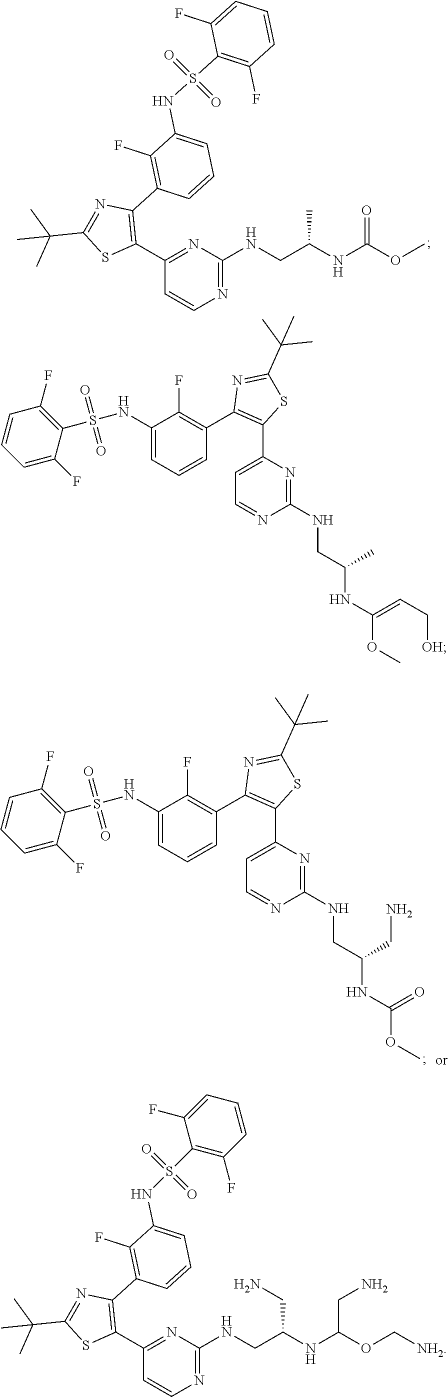

19. The compound or salt of claim 1, wherein the compound is

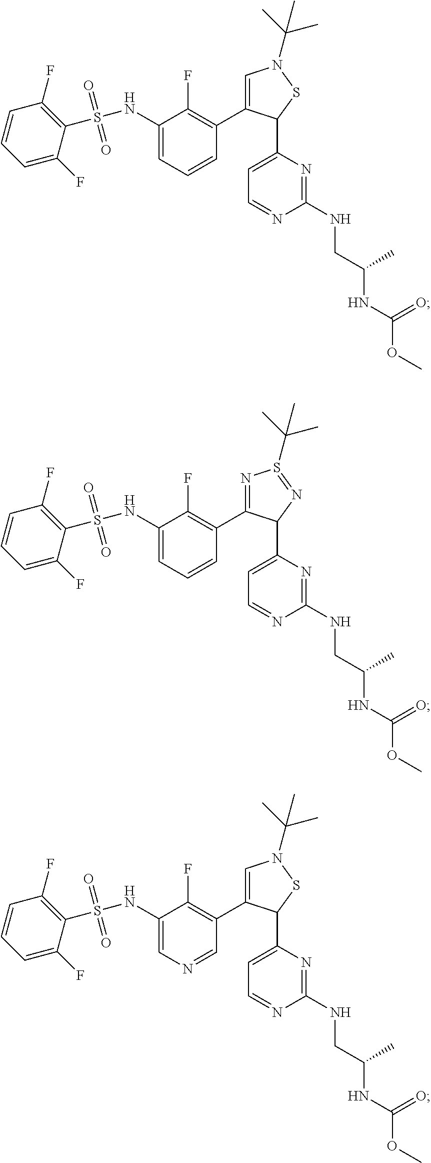 [SMILES: COC(=O)N[C@@H](C)CNc1nccc(C2SN(C(C)(C)C)C=C2c2cccc(NS(=O)(=O)c3c(F)cccc3F)c2F)n1 ; COC(=O)N[C@@H](C)CNc1nccc(C2N=S(C(C)(C)C)N=C2c2cccc(NS(=O)(=O)c3c(F)cccc3F)c2F)n1 ; COC(=O)N[C@@H](C)CNc1nccc(C2SN(C(C)(C)C)C=C2c2cncc(NS(=O)(=O)c3c(F)cccc3F)c2F)n1]

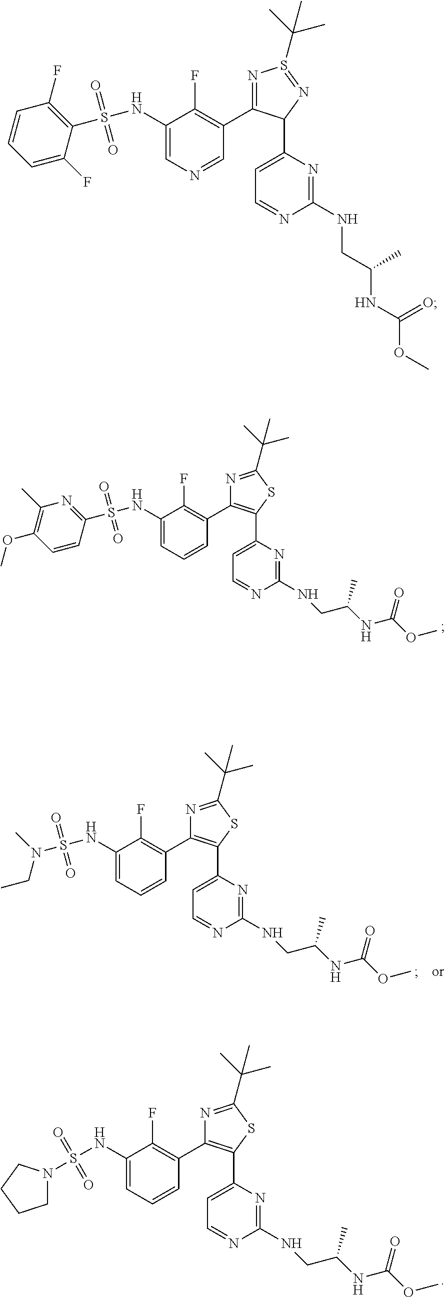

20. The compound or salt of claim 3, wherein the compound is

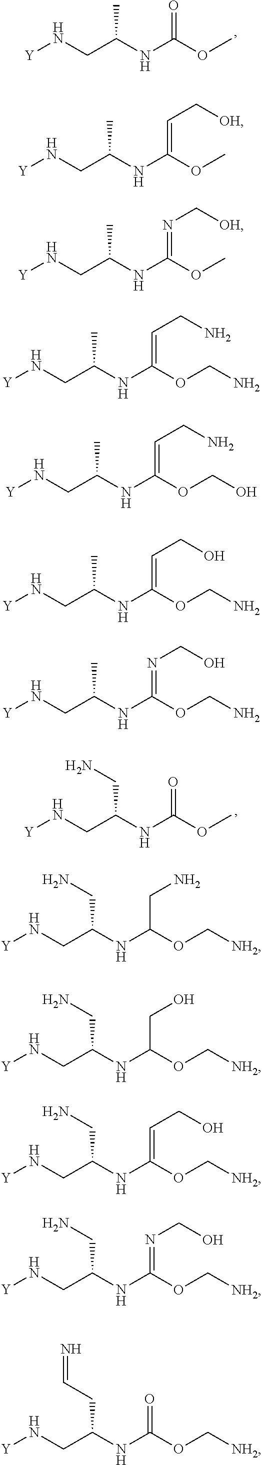

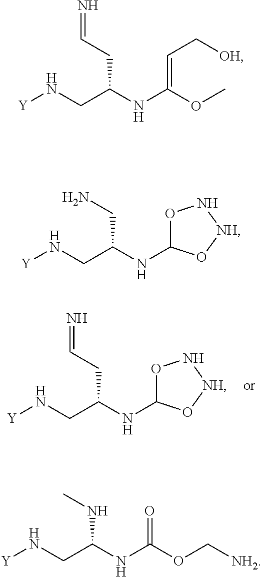

21. A pharmaceutical composition comprising a compound of claim 1 or a pharmaceutically acceptable salt thereof, together with a pharmaceutically acceptable carrier.

22. A method of treating a patient suffering from melanoma, thyroid cancer, hairy cell leukemia, ovarian cancer, lung cancer, pancreatic, prostate, gastric, endometrial, glioblastomas, astrocytomas, or colorectal cancer comprising administering a therapeutically effective amount of a compound or salt thereof of claim 1 to the patient.

23. A method of treating a patient suffering from a cancer susceptible to treatment with a RAF inhibitor, comprising administering a therapeutically effective amount of a compound or salt thereof of claim 1 to the patient.

24. A method of treating a patient suffering from a cancer, comprising
(a) determining that a cell of the cancer contains a BRAF^{V600E} mutation, and
(b) administering a therapeutically effective amount of a compound or salt thereof of claim 1 to the patient.

25. The method of claim 22, wherein the compound or salt thereof of claim 1 is a first active agent and is administered together with at least one additional active agent.

26. The method of claim 25, wherein the additional active agent is a RAF inhibitor, a MEK inhibitor, an ERK inhibitor, an RTK inhibitor, a SHP2 inhibitor, a KRAS or NRAS mutant inhibitor, a CDK4/6 inhibitor or a PI3K inhibitor.

27-28. (canceled)
